SEQUENCE LISTING (316 sequences)

SEQ ID NO: 1 - RNA, 19 nt, synthetic construct
  source 1..19: mol_type = other RNA
  misc_feature 1..19: note = siRNA strand - modified as per Table 2
caagcatggagcggcttta

SEQ ID NO: 2 - RNA, 21 nt, synthetic construct
  source 1..21: mol_type = other RNA
  misc_feature 1..21: note = siRNA strand - modified as per Table 2
taaagccgctccatgcttgtg

SEQ ID NO: 3 - RNA, 19 nt, synthetic construct
  source 1..19: mol_type = other RNA
  misc_feature 1..19: note = siRNA strand - modified as per Table 2
ttccagagagcgcaacaaa

SEQ ID NO: 4 - RNA, 21 nt, synthetic construct
  source 1..21: mol_type = other RNA
  misc_feature 1..21: note = siRNA strand - modified as per Table 2
tttgttgcgctctctggaata

SEQ ID NO: 5 - RNA, 19 nt, synthetic construct
  source 1..19: mol_type = other RNA
  misc_feature 1..19: note = siRNA strand - modified as per Table 2
tccagagagcgcaacaaca

SEQ ID NO: 6 - RNA, 21 nt, synthetic construct
  source 1..21: mol_type = other RNA
  misc_feature 1..21: note = siRNA strand - modified as per Table 2
tgttgttgcgctctctggaat

SEQ ID NO: 7 - RNA, 19 nt, synthetic construct
  source 1..19: mol_type = other RNA
  misc_feature 1..19: note = siRNA strand - modified as per Table 2
ccagagagcgcaacaacta

SEQ ID NO: 8 - RNA, 21 nt, synthetic construct
  source 1..21: mol_type = other RNA
  misc_feature 1..21: note = siRNA strand - modified as per Table 2
tagttgttgcgctctctggaa

SEQ ID NO: 9 - RNA, 19 nt, synthetic construct
  source 1..19: mol_type = other RNA
  misc_feature 1..19: note = siRNA strand - modified as per Table 2
cagagagcgcaacaactga

SEQ ID NO: 10 - RNA, 21 nt, synthetic construct
  source 1..21: mol_type = other RNA
  misc_feature 1..21: note = siRNA strand - modified as per Table 2
tcagttgttgcgctctctgga

SEQ ID NO: 11 - RNA, 19 nt, synthetic construct
  source 1..19: mol_type = other RNA
  misc_feature 1..19: note = siRNA strand - modified as per Table 2
agagagcgcaacaactgta

SEQ ID NO: 12 - RNA, 21 nt, synthetic construct
  source 1..21: mol_type = other RNA
  misc_feature 1..21: note = siRNA strand - modified as per Table 2
tacagttgttgcgctctctgg

SEQ ID NO: 13 - RNA, 19 nt, synthetic construct
  source 1..19: mol_type = other RNA
  misc_feature 1..19: note = siRNA strand - modified as per Table 2
cccaagatcctctcagaca

SEQ ID NO: 14 - RNA, 21 nt, synthetic construct
  source 1..21: mol_type = other RNA
  misc_feature 1..21: note = siRNA strand - modified as per Table 2
tgtctgagaggatcttgggga

SEQ ID NO: 15 - RNA, 19 nt, synthetic construct
  source 1..19: mol_type = other RNA
  misc_feature 1..19: note = siRNA strand - modified as per Table 2
agatcctctcagaccgtca

SEQ ID NO: 16 - RNA, 21 nt, synthetic construct
  source 1..21: mol_type = other RNA
  misc_feature 1..21: note = siRNA strand - modified as per Table 2
tgacggtctgagaggatcttg

SEQ ID NO: 17 - RNA, 19 nt, synthetic construct
  source 1..19: mol_type = other RNA
  misc_feature 1..19: note = siRNA strand - modified as per Table 2
ctctcagaccgtctaccaa

SEQ ID NO: 18 - RNA, 21 nt, synthetic construct
  source 1..21: mol_type = other RNA
  misc_feature 1..21: note = siRNA strand - modified as per Table 2
ttggtagacggtctgagagga

SEQ ID NO: 19 - RNA, 19 nt, synthetic construct
  source 1..19: mol_type = other RNA
  misc_feature 1..19: note = siRNA strand - modified as per Table 2
tctcagaccgtctaccaga

SEQ ID NO: 20 - RNA, 21 nt, synthetic construct
  source 1..21: mol_type = other RNA
  misc_feature 1..21: note = siRNA strand - modified as per Table 2
tctggtagacggtctgagagg

SEQ ID NO: 21 - RNA, 19 nt, synthetic construct
  source 1..19: mol_type = other RNA
  misc_feature 1..19: note = siRNA strand - modified as per Table 2
ctcagaccgtctaccagca

SEQ ID NO: 22 - RNA, 21 nt, synthetic construct
  source 1..21: mol_type = other RNA
  misc_feature 1..21: note = siRNA strand - modified as per Table 2
tgctggtagacggtctgagag

SEQ ID NO: 23 - RNA, 19 nt, synthetic construct
  source 1..19: mol_type = other RNA
  misc_feature 1..19: note = siRNA strand - modified as per Table 2
caggcagccaaacctcata

SEQ ID NO: 24 - RNA, 21 nt, synthetic construct
  source 1..21: mol_type = other RNA
  misc_feature 1..21: note = siRNA strand - modified as per Table 2
tatgaggtttggctgcctggg

SEQ ID NO: 25 - RNA, 19 nt, synthetic construct
  source 1..19: mol_type = other RNA
  misc_feature 1..19: note = siRNA strand - modified as per Table 2
tgttggtggtcagtgagga

SEQ ID NO: 26 - RNA, 21 nt, synthetic construct
  source 1..21: mol_type = other RNA
  misc_feature 1..21: note = siRNA strand - modified as per Table 2
tcctcactgaccaccaacaca

SEQ ID NO: 27 - RNA, 19 nt, synthetic construct
  source 1..19: mol_type = other RNA
  misc_feature 1..19: note = siRNA strand - modified as per Table 2
tgtcaactatgagcctgaa

SEQ ID NO: 28 - RNA, 21 nt, synthetic construct
  source 1..21: mol_type = other RNA
  misc_feature 1..21: note = siRNA strand - modified as per Table 2
ttcaggctcatagttgacatc

SEQ ID NO: 29 - RNA, 19 nt, synthetic construct
  source 1..19: mol_type = other RNA
  misc_feature 1..19: note = siRNA strand - modified as per Table 2
caactatgagcctgaccta

SEQ ID NO: 30 - RNA, 21 nt, synthetic construct
  source 1..21: mol_type = other RNA
  misc_feature 1..21: note = siRNA strand - modified as per Table 2
taggtcaggctcatagttgac

SEQ ID NO: 31 - RNA, 19 nt, synthetic construct
  source 1..19: mol_type = other RNA
  misc_feature 1..19: note = siRNA strand - modified as per Table 2
ctatgagcctgacctggaa

SEQ ID NO: 32 - RNA, 21 nt, synthetic construct
  source 1..21: mol_type = other RNA
  misc_feature 1..21: note = siRNA strand - modified as per Table 2
ttccaggtcaggctcatagtt

SEQ ID NO: 33 - RNA, 19 nt, synthetic construct
  source 1..19: mol_type = other RNA
  misc_feature 1..19: note = siRNA strand - modified as per Table 2
ggaggaccctgacgtctaa

SEQ ID NO: 34 - RNA, 21 nt, synthetic construct
  source 1..21: mol_type = other RNA
  misc_feature 1..21: note = siRNA strand - modified as per Table 2
ttagacgtcagggtcctccag

SEQ ID NO: 35 - RNA, 19 nt, synthetic construct
  source 1..19: mol_type = other RNA
  misc_feature 1..19: note = siRNA strand - modified as per Table 2
ggtgatcagcttcgcccaa

SEQ ID NO: 36 - RNA, 21 nt, synthetic construct
  source 1..21: mol_type = other RNA
  misc_feature 1..21: note = siRNA strand - modified as per Table 2
ttgggcgaagctgatcacccc

SEQ ID NO: 37 - RNA, 19 nt, synthetic construct
  source 1..19: mol_type = other RNA
  misc_feature 1..19: note = siRNA strand - modified as per Table 2
cccagaaggtgaccaatga

SEQ ID NO: 38 - RNA, 21 nt, synthetic construct
  source 1..21: mol_type = other RNA
  misc_feature 1..21: note = siRNA strand - modified as per Table 2
tcattggtcaccttctgggcg

SEQ ID NO: 39 - RNA, 19 nt, synthetic construct
  source 1..19: mol_type = other RNA
  misc_feature 1..19: note = siRNA strand - modified as per Table 2
agaaggtgaccaatgctca

SEQ ID NO: 40 - RNA, 21 nt, synthetic construct
  source 1..21: mol_type = other RNA
  misc_feature 1..21: note = siRNA strand - modified as per Table 2
tgagcattggtcaccttctgg

SEQ ID NO: 41 - RNA, 19 nt, synthetic construct
  source 1..19: mol_type = other RNA
  misc_feature 1..19: note = siRNA strand - modified as per Table 2
ggtgaccaatgctcaggaa

SEQ ID NO: 42 - RNA, 21 nt, synthetic construct
  source 1..21: mol_type = other RNA
  misc_feature 1..21: note = siRNA strand - modified as per Table 2
ttcctgagcattggtcacctt

SEQ ID NO: 43 - RNA, 19 nt, synthetic construct
  source 1..19: mol_type = other RNA
  misc_feature 1..19: note = siRNA strand - modified as per Table 2
gaccaatgctcaggactta

SEQ ID NO: 44 - RNA, 21 nt, synthetic construct
  source 1..21: mol_type = other RNA
  misc_feature 1..21: note = siRNA strand - modified as per Table 2
taagtcctgagcattggtcac

SEQ ID NO: 45 - RNA, 19 nt, synthetic construct
  source 1..19: mol_type = other RNA
  misc_feature 1..19: note = siRNA strand - modified as per Table 2
agtgtatggacatgtgcaa

SEQ ID NO: 46 - RNA, 21 nt, synthetic construct
  source 1..21: mol_type = other RNA
  misc_feature 1..21: note = siRNA strand - modified as per Table 2
ttgcacatgtccatacactgc

SEQ ID NO: 47 - RNA, 19 nt, synthetic construct
  source 1..19: mol_type = other RNA
  misc_feature 1..19: note = siRNA strand - modified as per Table 2
ttccttcaatcaaacccaa

SEQ ID NO: 48 - RNA, 21 nt, synthetic construct
  source 1..21: mol_type = other RNA
  misc_feature 1..21: note = siRNA strand - modified as per Table 2
ttgggtttgattgaaggaagg

SEQ ID NO: 49 - RNA, 19 nt, synthetic construct
  source 1..19: mol_type = other RNA
  misc_feature 1..19: note = siRNA strand - modified as per Table 2
gtccttcaatcaaacccaa

SEQ ID NO: 50 - RNA, 21 nt, synthetic construct
  source 1..21: mol_type = other RNA
  misc_feature 1..21: note = siRNA strand - modified as per Table 2
ttgggtttgattgaaggacgg

SEQ ID NO: 51 - RNA, 19 nt, synthetic construct
  source 1..19: mol_type = other RNA
  misc_feature 1..19: note = siRNA strand - modified as per Table 2
ccttcaatcaaacccagta

SEQ ID NO: 52 - RNA, 21 nt, synthetic construct
  source 1..21: mol_type = other RNA
  misc_feature 1..21: note = siRNA strand - modified as per Table 2
tactgggtttgattgaaggaa

SEQ ID NO: 53 - RNA, 19 nt, synthetic construct
  source 1..19: mol_type = other RNA
  misc_feature 1..19: note = siRNA strand - modified as per Table 2
cttcaatcaaacccagtta

SEQ ID NO: 54 - RNA, 21 nt, synthetic construct
  source 1..21: mol_type = other RNA
  misc_feature 1..21: note = siRNA strand - modified as per Table 2
taactgggtttgattgaagga

SEQ ID NO: 55 - RNA, 19 nt, synthetic construct
  source 1..19: mol_type = other RNA
  misc_feature 1..19: note = siRNA strand - modified as per Table 2
ttcaatcaaacccagttca

SEQ ID NO: 56 - RNA, 21 nt, synthetic construct
  source 1..21: mol_type = other RNA
  misc_feature 1..21: note = siRNA strand - modified as per Table 2
tgaactgggtttgattgaagg

SEQ ID NO: 57 - RNA, 19 nt, synthetic construct
  source 1..19: mol_type = other RNA
  misc_feature 1..19: note = siRNA strand - modified as per Table 2
ctcagagccagatcactaa

SEQ ID NO: 58 - RNA, 21 nt, synthetic construct
  source 1..21: mol_type = other RNA
  misc_feature 1..21: note = siRNA strand - modified as per Table 2
ttagtgatctggctctgagcg

SEQ ID NO: 59 - RNA, 19 nt, synthetic construct
  source 1..19: mol_type = other RNA
  misc_feature 1..19: note = siRNA strand - modified as per Table 2
cgcagaagtctcctcttca

SEQ ID NO: 60 - RNA, 21 nt, synthetic construct
  source 1..21: mol_type = other RNA
  misc_feature 1..21: note = siRNA strand - modified as per Table 2
tgaagaggagacttctgcggg

SEQ ID NO: 61 - RNA, 19 nt, synthetic construct
  source 1..19: mol_type = other RNA
  misc_feature 1..19: note = siRNA strand - modified as per Table 2
agaagtctcctcttcatca

SEQ ID NO: 62 - RNA, 21 nt, synthetic construct
  source 1..21: mol_type = other RNA
  misc_feature 1..21: note = siRNA strand - modified as per Table 2
tgatgaagaggagacttctgc

SEQ ID NO: 63 - RNA, 19 nt, synthetic construct
  source 1..19: mol_type = other RNA
  misc_feature 1..19: note = siRNA strand - modified as per Table 2
gctgcacctcaaagccgta

SEQ ID NO: 64 - RNA, 21 nt, synthetic construct
  source 1..21: mol_type = other RNA
  misc_feature 1..21: note = siRNA strand - modified as per Table 2
tacggctttgaggtgcagcac

SEQ ID NO: 65 - RNA, 19 nt, synthetic construct
  source 1..19: mol_type = other RNA
  misc_feature 1..19: note = siRNA strand - modified as per Table 2
gcacctcaaagccgtagta

SEQ ID NO: 66 - RNA, 21 nt, synthetic construct
  source 1..21: mol_type = other RNA
  misc_feature 1..21: note = siRNA strand - modified as per Table 2
tactacggctttgaggtgcag

SEQ ID NO: 67 - RNA, 19 nt, synthetic construct
  source 1..19: mol_type = other RNA
  misc_feature 1..19: note = siRNA strand - modified as per Table 2
acctcaaagccgtagtgta

SEQ ID NO: 68 - RNA, 21 nt, synthetic construct
  source 1..21: mol_type = other RNA
  misc_feature 1..21: note = siRNA strand - modified as per Table 2
tacactacggctttgaggtgc

SEQ ID NO: 69 - RNA, 19 nt, synthetic construct
  source 1..19: mol_type = other RNA
  misc_feature 1..19: note = siRNA strand - modified as per Table 2
cctcaaagccgtagtgtaa

SEQ ID NO: 70 - RNA, 21 nt, synthetic construct
  source 1..21: mol_type = other RNA
  misc_feature 1..21: note = siRNA strand - modified as per Table 2
ttacactacggctttgaggtg

SEQ ID NO: 71 - RNA, 19 nt, synthetic construct
  source 1..19: mol_type = other RNA
  misc_feature 1..19: note = siRNA strand - modified as per Table 2
tcaaagccgtagtgtacga

SEQ ID NO: 72 - RNA, 21 nt, synthetic construct
  source 1..21: mol_type = other RNA
  misc_feature 1..21: note = siRNA strand - modified as per Table 2
tcgtacactacggctttgagg

SEQ ID NO: 73 - RNA, 19 nt, synthetic construct
  source 1..19: mol_type = other RNA
  misc_feature 1..19: note = siRNA strand - modified as per Table 2
aagccgtagtgtacgtgaa

SEQ ID NO: 74 - RNA, 21 nt, synthetic construct
  source 1..21: mol_type = other RNA
  misc_feature 1..21: note = siRNA strand - modified as per Table 2
ttcacgtacactacggctttg

SEQ ID NO: 75 - RNA, 19 nt, synthetic construct
  source 1..19: mol_type = other RNA
  misc_feature 1..19: note = siRNA strand - modified as per Table 2
cgtagtgtacgtgagccta

SEQ ID NO: 76 - RNA, 21 nt, synthetic construct
  source 1..21: mol_type = other RNA
  misc_feature 1..21: note = siRNA strand - modified as per Table 2
taggctcacgtacactacggc

SEQ ID NO: 77 - RNA, 19 nt, synthetic construct
  source 1..19: mol_type = other RNA
  misc_feature 1..19: note = siRNA strand - modified as per Table 2
agtgtacgtgagcctggaa

SEQ ID NO: 78 - RNA, 21 nt, synthetic construct
  source 1..21: mol_type = other RNA
  misc_feature 1..21: note = siRNA strand - modified as per Table 2
ttccaggctcacgtacactac

SEQ ID NO: 79 - RNA, 19 nt, synthetic construct
  source 1..19: mol_type = other RNA
  misc_feature 1..19: note = siRNA strand - modified as per Table 2
gggatgacaagtttcatga

SEQ ID NO: 80 - RNA, 21 nt, synthetic construct
  source 1..21: mol_type = other RNA
  misc_feature 1..21: note = siRNA strand - modified as per Table 2
tcatgaaacttgtcatccccc

SEQ ID NO: 81 - RNA, 19 nt, synthetic construct
  source 1..19: mol_type = other RNA
  misc_feature 1..19: note = siRNA strand - modified as per Table 2
caagtttcatgccaagaca

SEQ ID NO: 82 - RNA, 21 nt, synthetic construct
  source 1..21: mol_type = other RNA
  misc_feature 1..21: note = siRNA strand - modified as per Table 2
tgtcttggcatgaaacttgtc

SEQ ID NO: 83 - RNA, 19 nt, synthetic construct
  source 1..19: mol_type = other RNA
  misc_feature 1..19: note = siRNA strand - modified as per Table 2
ctgacaagtctcattgaga

SEQ ID NO: 84 - RNA, 21 nt, synthetic construct
  source 1..21: mol_type = other RNA
  misc_feature 1..21: note = siRNA strand - modified as per Table 2
tctcaatgagacttgtcagaa

SEQ ID NO: 85 - RNA, 19 nt, synthetic construct
  source 1..19: mol_type = other RNA
  misc_feature 1..19: note = siRNA strand - modified as per Table 2
tgacaagtctcattgagaa

SEQ ID NO: 86 - RNA, 21 nt, synthetic construct
  source 1..21: mol_type = other RNA
  misc_feature 1..21: note = siRNA strand - modified as per Table 2
ttctcaatgagacttgtcaga

SEQ ID NO: 87 - RNA, 19 nt, synthetic construct
  source 1..19: mol_type = other RNA
  misc_feature 1..19: note = siRNA strand - modified as per Table 2
acaagtctcattgagagta

SEQ ID NO: 88 - RNA, 21 nt, synthetic construct
  source 1..21: mol_type = other RNA
  misc_feature 1..21: note = siRNA strand - modified as per Table 2
tactctcaatgagacttgtca

SEQ ID NO: 89 - RNA, 19 nt, synthetic construct
  source 1..19: mol_type = other RNA
  misc_feature 1..19: note = siRNA strand - modified as per Table 2
tcattgagagtgtcctgaa

SEQ ID NO: 90 - RNA, 21 nt, synthetic construct
  source 1..21: mol_type = other RNA
  misc_feature 1..21: note = siRNA strand - modified as per Table 2
ttcaggacactctcaatgaga

SEQ ID NO: 91 - RNA, 19 nt, synthetic construct
  source 1..19: mol_type = other RNA
  misc_feature 1..19: note = siRNA strand - modified as per Table 2
cattgagagtgtcctgaaa

SEQ ID NO: 92 - RNA, 21 nt, synthetic construct
  source 1..21: mol_type = other RNA
  misc_feature 1..21: note = siRNA strand - modified as per Table 2
tttcaggacactctcaatgag

SEQ ID NO: 93 - RNA, 19 nt, synthetic construct
  source 1..19: mol_type = other RNA
  misc_feature 1..19: note = siRNA strand - modified as per Table 2
gggcagactctctatgaaa

SEQ ID NO: 94 - RNA, 21 nt, synthetic construct
  source 1..21: mol_type = other RNA
  misc_feature 1..21: note = siRNA strand - modified as per Table 2
tttcatagagagtctgcccac

SEQ ID NO: 95 - RNA, 19 nt, synthetic construct
  source 1..19: mol_type = other RNA
  misc_feature 1..19: note = siRNA strand - modified as per Table 2
ggcagactctctatgaaca

SEQ ID NO: 96 - RNA, 21 nt, synthetic construct
  source 1..21: mol_type = other RNA
  misc_feature 1..21: note = siRNA strand - modified as per Table 2
tgttcatagagagtctgccca

SEQ ID NO: 97 - RNA, 19 nt, synthetic construct
  source 1..19: mol_type = other RNA
  misc_feature 1..19: note = siRNA strand - modified as per Table 2
gactctctatgaacaggta

SEQ ID NO: 98 - RNA, 21 nt, synthetic construct
  source 1..21: mol_type = other RNA
  misc_feature 1..21: note = siRNA strand - modified as per Table 2
tacctgttcatagagagtctg

SEQ ID NO: 99 - RNA, 19 nt, synthetic construct
  source 1..19: mol_type = other RNA
  misc_feature 1..19: note = siRNA strand - modified as per Table 2
aacaggtggtgttcaccaa

SEQ ID NO: 100 - RNA, 21 nt, synthetic construct
  source 1..21: mol_type = other RNA
  misc_feature 1..21: note = siRNA strand - modified as per Table 2
ttggtgaacaccacctgttca

SEQ ID NO: 101 - RNA, 19 nt, synthetic construct
  source 1..19: mol_type = other RNA
  misc_feature 1..19: note = siRNA strand - modified as per Table 2
acacaaaggaggacactta

SEQ ID NO: 102 - RNA, 21 nt, synthetic construct
  source 1..21: mol_type = other RNA
  misc_feature 1..21: note = siRNA strand - modified as per Table 2
taagtgtcctcctttgtgtgc

SEQ ID NO: 103 - RNA, 19 nt, synthetic construct
  source 1..19: mol_type = other RNA
  misc_feature 1..19: note = siRNA strand - modified as per Table 2
cacaaaggaggacacttaa

SEQ ID NO: 104 - RNA, 21 nt, synthetic construct
  source 1..21: mol_type = other RNA
  misc_feature 1..21: note = siRNA strand - modified as per Table 2
ttaagtgtcctcctttgtgtg

SEQ ID NO: 105 - RNA, 19 nt, synthetic construct
  source 1..19: mol_type = other RNA
  misc_feature 1..19: note = siRNA strand - modified as per Table 2
aaaggaggacacttatgaa

SEQ ID NO: 106 - RNA, 21 nt, synthetic construct
  source 1..21: mol_type = other RNA
  misc_feature 1..21: note = siRNA strand - modified as per Table 2
ttcataagtgtcctcctttgt

SEQ ID NO: 107 - RNA, 19 nt, synthetic construct
  source 1..19: mol_type = other RNA
  misc_feature 1..19: note = siRNA strand - modified as per Table 2
gaaggaggacacttatgaa

SEQ ID NO: 108 - RNA, 21 nt, synthetic construct
  source 1..21: mol_type = other RNA
  misc_feature 1..21: note = siRNA strand - modified as per Table 2
ttcataagtgtcctccttcgt

SEQ ID NO: 109 - RNA, 19 nt, synthetic construct
  source 1..19: mol_type = other RNA
  misc_feature 1..19: note = siRNA strand - modified as per Table 2
gcacatcgggaacctcaaa

SEQ ID NO: 110 - RNA, 21 nt, synthetic construct
  source 1..21: mol_type = other RNA
  misc_feature 1..21: note = siRNA strand - modified as per Table 2
tttgaggttcccgatgtgcct

SEQ ID NO: 111 - RNA, 19 nt, synthetic construct
  source 1..19: mol_type = other RNA
  misc_feature 1..19: note = siRNA strand - modified as per Table 2
tcgggaacctcaacgagta

SEQ ID NO: 112 - RNA, 21 nt, synthetic construct
  source 1..21: mol_type = other RNA
  misc_feature 1..21: note = siRNA strand - modified as per Table 2
tactcgttgaggttcccgatg

SEQ ID NO: 113 - RNA, 19 nt, synthetic construct
  source 1..19: mol_type = other RNA
  misc_feature 1..19: note = siRNA strand - modified as per Table 2
gcaggagatctacagctca

SEQ ID NO: 114 - RNA, 21 nt, synthetic construct
  source 1..21: mol_type = other RNA
  misc_feature 1..21: note = siRNA strand - modified as per Table 2
tgagctgtagatctcctgccg

SEQ ID NO: 115 - RNA, 19 nt, synthetic construct
  source 1..19: mol_type = other RNA
  misc_feature 1..19: note = siRNA strand - modified as per Table 2
gagagacgagcgactgaca

SEQ ID NO: 116 - RNA, 21 nt, synthetic construct
  source 1..21: mol_type = other RNA
  misc_feature 1..21: note = siRNA strand - modified as per Table 2
tgtcagtcgctcgtctctctc

SEQ ID NO: 117 - RNA, 19 nt, synthetic construct
  source 1..19: mol_type = other RNA
  misc_feature 1..19: note = siRNA strand - modified as per Table 2
agagacgagcgactgacaa

SEQ ID NO: 118 - RNA, 21 nt, synthetic construct
  source 1..21: mol_type = other RNA
  misc_feature 1..21: note = siRNA strand - modified as per Table 2
ttgtcagtcgctcgtctctct

SEQ ID NO: 119 - RNA, 19 nt, synthetic construct
  source 1..19: mol_type = other RNA
  misc_feature 1..19: note = siRNA strand - modified as per Table 2
gagacgagcgactgacaca

SEQ ID NO: 120 - RNA, 21 nt, synthetic construct
  source 1..21: mol_type = other RNA
  misc_feature 1..21: note = siRNA strand - modified as per Table 2
tgtgtcagtcgctcgtctctc

SEQ ID NO: 121 - RNA, 19 nt, synthetic construct
  source 1..19: mol_type = other RNA
  misc_feature 1..19: note = siRNA strand - modified as per Table 2
tgacacgcatgtacaacga

SEQ ID NO: 122 - RNA, 21 nt, synthetic construct
  source 1..21: mol_type = other RNA
  misc_feature 1..21: note = siRNA strand - modified as per Table 2
tcgttgtacatgcgtgtcagt

SEQ ID NO: 123 - RNA, 19 nt, synthetic construct
  source 1..19: mol_type = other RNA
  misc_feature 1..19: note = siRNA strand - modified as per Table 2
gcatgtacaacgtgcgcaa

SEQ ID NO: 124 - RNA, 21 nt, synthetic construct
  source 1..21: mol_type = other RNA
  misc_feature 1..21: note = siRNA strand - modified as per Table 2
ttgcgcacgttgtacatgcgt

SEQ ID NO: 125 - RNA, 19 nt, synthetic construct
  source 1..19: mol_type = other RNA
  misc_feature 1..19: note = siRNA strand - modified as per Table 2
tgtacaacgtgcgcataaa

SEQ ID NO: 126 - RNA, 21 nt, synthetic construct
  source 1..21: mol_type = other RNA
  misc_feature 1..21: note = siRNA strand - modified as per Table 2
tttatgcgcacgttgtacatg

SEQ ID NO: 127 - RNA, 19 nt, synthetic construct
  source 1..19: mol_type = other RNA
  misc_feature 1..19: note = siRNA strand - modified as per Table 2
gtacaacgtgcgcataata

SEQ ID NO: 128 - RNA, 21 nt, synthetic construct
  source 1..21: mol_type = other RNA
  misc_feature 1..21: note = siRNA strand - modified as per Table 2
tattatgcgcacgttgtacat

SEQ ID NO: 129 - RNA, 19 nt, synthetic construct
  source 1..19: mol_type = other RNA
  misc_feature 1..19: note = siRNA strand - modified as per Table 2
cgcataatgcgggtggaga

SEQ ID NO: 130 - RNA, 21 nt, synthetic construct
  source 1..21: mol_type = other RNA
  misc_feature 1..21: note = siRNA strand - modified as per Table 2
tctccacccgcattatgcgca

SEQ ID NO: 131 - RNA, 19 nt, synthetic construct
  source 1..19: mol_type = other RNA
  misc_feature 1..19: note = siRNA strand - modified as per Table 2
gcataatgcgggtggagta

SEQ ID NO: 132 - RNA, 21 nt, synthetic construct
  source 1..21: mol_type = other RNA
  misc_feature 1..21: note = siRNA strand - modified as per Table 2
tactccacccgcattatgcgc

SEQ ID NO: 133 - RNA, 19 nt, synthetic construct
  source 1..19: mol_type = other RNA
  misc_feature 1..19: note = siRNA strand - modified as per Table 2
taatgcgggtggagttcta

SEQ ID NO: 134 - RNA, 21 nt, synthetic construct
  source 1..21: mol_type = other RNA
  misc_feature 1..21: note = siRNA strand - modified as per Table 2
tagaactccacccgcattatg

SEQ ID NO: 135 - RNA, 19 nt, synthetic construct
  source 1..19: mol_type = other RNA
  misc_feature 1..19: note = siRNA strand - modified as per Table 2
tgcgggtggagttctacta

SEQ ID NO: 136 - RNA, 21 nt, synthetic construct
  source 1..21: mol_type = other RNA
  misc_feature 1..21: note = siRNA strand - modified as per Table 2
tagtagaactccacccgcatt

SEQ ID NO: 137 - RNA, 19 nt, synthetic construct
  source 1..19: mol_type = other RNA
  misc_feature 1..19: note = siRNA strand - modified as per Table 2
gcgggtggagttctactta

SEQ ID NO: 138 - RNA, 21 nt, synthetic construct
  source 1..21: mol_type = other RNA
  misc_feature 1..21: note = siRNA strand - modified as per Table 2
taagtagaactccacccgcat

SEQ ID NO: 139 - RNA, 19 nt, synthetic construct
  source 1..19: mol_type = other RNA
  misc_feature 1..19: note = siRNA strand - modified as per Table 2
cgggtggagttctacttca

SEQ ID NO: 140 - RNA, 21 nt, synthetic construct
  source 1..21: mol_type = other RNA
  misc_feature 1..21: note = siRNA strand - modified as per Table 2
tgaagtagaactccacccgca

SEQ ID NO: 141 - RNA, 19 nt, synthetic construct
  source 1..19: mol_type = other RNA
  misc_feature 1..19: note = siRNA strand - modified as per Table 2
tggagttctacttccttta

SEQ ID NO: 142 - RNA, 21 nt, synthetic construct
  source 1..21: mol_type = other RNA
  misc_feature 1..21: note = siRNA strand - modified as per Table 2
taaaggaagtagaactccacc

SEQ ID NO: 143 - RNA, 19 nt, synthetic construct
  source 1..19: mol_type = other RNA
  misc_feature 1..19: note = siRNA strand - modified as per Table 2
ctacttcctttcccagtaa

SEQ ID NO: 144 - RNA, 21 nt, synthetic construct
  source 1..21: mol_type = other RNA
  misc_feature 1..21: note = siRNA strand - modified as per Table 2
ttactgggaaaggaagtagaa

SEQ ID NO: 145 - RNA, 19 nt, synthetic construct
  source 1..19: mol_type = other RNA
  misc_feature 1..19: note = siRNA strand - modified as per Table 2
ttcctttcccagtacgtga

SEQ ID NO: 146 - RNA, 21 nt, synthetic construct
  source 1..21: mol_type = other RNA
  misc_feature 1..21: note = siRNA strand - modified as per Table 2
tcacgtactgggaaaggaagt

SEQ ID NO: 147 - RNA, 19 nt, synthetic construct
  source 1..19: mol_type = other RNA
  misc_feature 1..19: note = siRNA strand - modified as per Table 2
gtcctttcccagtacgtga

SEQ ID NO: 148 - RNA, 21 nt, synthetic construct
  source 1..21: mol_type = other RNA
  misc_feature 1..21: note = siRNA strand - modified as per Table 2
tcacgtactgggaaaggacgt

SEQ ID NO: 149 - RNA, 19 nt, synthetic construct
  source 1..19: mol_type = other RNA
  misc_feature 1..19: note = siRNA strand - modified as per Table 2
tcctttcccagtacgtgta

SEQ ID NO: 150 - RNA, 21 nt, synthetic construct
  source 1..21: mol_type = other RNA
  misc_feature 1..21: note = siRNA strand - modified as per Table 2
tacacgtactgggaaaggaag

SEQ ID NO: 151 - RNA, 19 nt, synthetic construct
  source 1..19: mol_type = other RNA
  misc_feature 1..19: note = siRNA strand - modified as per Table 2
agccaatgcgcttagcgga

SEQ ID NO: 152 - RNA, 21 nt, synthetic construct
  source 1..21: mol_type = other RNA
  misc_feature 1..21: note = siRNA strand - modified as per Table 2
tccgctaagcgcattggctgc

SEQ ID NO: 153 - RNA, 19 nt, synthetic construct
  source 1..19: mol_type = other RNA
  misc_feature 1..19: note = siRNA strand - modified as per Table 2
tctggaacattgataacaa

SEQ ID NO: 154 - RNA, 21 nt, synthetic construct
  source 1..21: mol_type = other RNA
  misc_feature 1..21: note = siRNA strand - modified as per Table 2
ttgttatcaatgttccagaca

SEQ ID NO: 155 - RNA, 19 nt, synthetic construct
  source 1..19: mol_type = other RNA
  misc_feature 1..19: note = siRNA strand - modified as per Table 2
ctggaacattgataacaaa

SEQ ID NO: 156 - RNA, 21 nt, synthetic construct
  source 1..21: mol_type = other RNA
  misc_feature 1..21: note = siRNA strand - modified as per Table 2
tttgttatcaatgttccagac

SEQ ID NO: 157 - RNA, 19 nt, synthetic construct
  source 1..19: mol_type = other RNA
  misc_feature 1..19: note = siRNA strand - modified as per Table 2
ggaacattgataacaacta

SEQ ID NO: 158 - RNA, 21 nt, synthetic construct
  source 1..21: mol_type = other RNA
  misc_feature 1..21: note = siRNA strand - modified as per Table 2
tagttgttatcaatgttccag

SEQ ID NO: 159 - RNA, 19 nt, synthetic construct
  source 1..19: mol_type = other RNA
  misc_feature 1..19: note = siRNA strand - modified as per Table 2
aacattgataacaacttca

SEQ ID NO: 160 - RNA, 21 nt, synthetic construct
  source 1..21: mol_type = other RNA
  misc_feature 1..21: note = siRNA strand - modified as per Table 2
tgaagttgttatcaatgttcc

SEQ ID NO: 161 - RNA, 19 nt, synthetic construct
  source 1..19: mol_type = other RNA
  misc_feature 1..19: note = siRNA strand - modified as per Table 2
gacattgataacaacttca

SEQ ID NO: 162 - RNA, 21 nt, synthetic construct
  source 1..21: mol_type = other RNA
  misc_feature 1..21: note = siRNA strand - modified as per Table 2
tgaagttgttatcaatgtccc

SEQ ID NO: 163 - RNA, 19 nt, synthetic construct
  source 1..19: mol_type = other RNA
  misc_feature 1..19: note = siRNA strand - modified as per Table 2
acattgataacaacttcta

SEQ ID NO: 164 - RNA, 21 nt, synthetic construct
  source 1..21: mol_type = other RNA
  misc_feature 1..21: note = siRNA strand - modified as per Table 2
tagaagttgttatcaatgttc

SEQ ID NO: 165 - RNA, 19 nt, synthetic construct
  source 1..19: mol_type = other RNA
  misc_feature 1..19: note = siRNA strand - modified as per Table 2
attgataacaacttctgaa

SEQ ID NO: 166 - RNA, 21 nt, synthetic construct
  source 1..21: mol_type = other RNA
  misc_feature 1..21: note = siRNA strand - modified as per Table 2
ttcagaagttgttatcaatgt

SEQ ID NO: 167 - RNA, 19 nt, synthetic construct
  source 1..19: mol_type = other RNA
  misc_feature 1..19: note = siRNA strand - modified as per Table 2
gttgataacaacttctgaa

SEQ ID NO: 168 - RNA, 21 nt, synthetic construct
  source 1..21: mol_type = other RNA
  misc_feature 1..21: note = siRNA strand - modified as per Table 2
ttcagaagttgttatcaacgt

SEQ ID NO: 169 - RNA, 19 nt, synthetic construct
  source 1..19: mol_type = other RNA
  misc_feature 1..19: note = siRNA strand - modified as per Table 2
ttgataacaacttctgaga

SEQ ID NO: 170 - RNA, 21 nt, synthetic construct
  source 1..21: mol_type = other RNA
  misc_feature 1..21: note = siRNA strand - modified as per Table 2
tctcagaagttgttatcaatg

SEQ ID NO: 171 - RNA, 19 nt, synthetic construct
  source 1..19: mol_type = other RNA
  misc_feature 1..19: note = siRNA strand - modified as per Table 2
gtgataacaacttctgaga

SEQ ID NO: 172 - RNA, 21 nt, synthetic construct
  source 1..21: mol_type = other RNA
  misc_feature 1..21: note = siRNA strand - modified as per Table 2
tctcagaagttgttatcactg

SEQ ID NO: 173 - RNA, 19 nt, synthetic construct
  source 1..19: mol_type = other RNA
  misc_feature 1..19: note = siRNA strand - modified as per Table 2
acctctcattgctgatcaa

SEQ ID NO: 174 - RNA, 21 nt, synthetic construct
  source 1..21: mol_type = other RNA
  misc_feature 1..21: note = siRNA strand - modified as per Table 2
ttgatcagcaatgagaggtgg

SEQ ID NO: 175 - RNA, 19 nt, synthetic construct
  source 1..19: mol_type = other RNA
  misc_feature 1..19: note = siRNA strand - modified as per Table 2
agcacagatatccacacaa

SEQ ID NO: 176 - RNA, 21 nt, synthetic construct
  source 1..21: mol_type = other RNA
  misc_feature 1..21: note = siRNA strand - modified as per Table 2
ttgtgtggatatctgtgctgg

SEQ ID NO: 177 - RNA, 19 nt, synthetic construct
  source 1..19: mol_type = other RNA
  misc_feature 1..19: note = siRNA strand - modified as per Table 2
cggtcatactgtcggttaa

SEQ ID NO: 178 - RNA, 21 nt, synthetic construct
  source 1..21: mol_type = other RNA
  misc_feature 1..21: note = siRNA strand - modified as per Table 2
ttaaccgacagtatgaccgtc

SEQ ID NO: 179 - RNA, 19 nt, synthetic construct
  source 1..19: mol_type = other RNA
  misc_feature 1..19: note = siRNA strand - modified as per Table 2
tactgtcggttaatcagaa

SEQ ID NO: 180 - RNA, 21 nt, synthetic construct
  source 1..21: mol_type = other RNA
  misc_feature 1..21: note = siRNA strand - modified as per Table 2
ttctgattaaccgacagtatg

SEQ ID NO: 181 - RNA, 19 nt, synthetic construct
  source 1..19: mol_type = other RNA
  misc_feature 1..19: note = siRNA strand - modified as per Table 2
gactgtcggttaatcagaa

SEQ ID NO: 182 - RNA, 21 nt, synthetic construct
  source 1..21: mol_type = other RNA
  misc_feature 1..21: note = siRNA strand - modified as per Table 2
ttctgattaaccgacagtctg

SEQ ID NO: 183 - RNA, 19 nt, synthetic construct
  source 1..19: mol_type = other RNA
  misc_feature 1..19: note = siRNA strand - modified as per Table 2
gggtcctccatacctagaa

SEQ ID NO: 184 - RNA, 21 nt, synthetic construct
  source 1..21: mol_type = other RNA
  misc_feature 1..21: note = siRNA strand - modified as per Table 2
ttctaggtatggaggacccca

SEQ ID NO: 185 - RNA, 19 nt, synthetic construct
  source 1..19: mol_type = other RNA
  misc_feature 1..19: note = siRNA strand - modified as per Table 2
agagatcgctggcaccata

SEQ ID NO: 186 - RNA, 21 nt, synthetic construct
  source 1..21: mol_type = other RNA
  misc_feature 1..21: note = siRNA strand - modified as per Table 2
tatggtgccagcgatctctcc

SEQ ID NO: 187 - RNA, 19 nt, synthetic construct
  source 1..19: mol_type = other RNA
  misc_feature 1..19: note = siRNA strand - modified as per Table 2
gagatcgctggcaccataa

SEQ ID NO: 188 - RNA, 21 nt, synthetic construct
  source 1..21: mol_type = other RNA
  misc_feature 1..21: note = siRNA strand - modified as per Table 2
ttatggtgccagcgatctctc

SEQ ID NO: 189 - RNA, 19 nt, synthetic construct
  source 1..19: mol_type = other RNA
  misc_feature 1..19: note = siRNA strand - modified as per Table 2
ctggcaccatagccttata

SEQ ID NO: 190 - RNA, 21 nt, synthetic construct
  source 1..21: mol_type = other RNA
  misc_feature 1..21: note = siRNA strand - modified as per Table 2
tataaggctatggtgccagcg

SEQ ID NO: 191 - RNA, 19 nt, synthetic construct
  source 1..19: mol_type = other RNA
  misc_feature 1..19: note = siRNA strand - modified as per Table 2
ccatagccttatggccaaa

SEQ ID NO: 192 - RNA, 21 nt, synthetic construct
  source 1..21: mol_type = other RNA
  misc_feature 1..21: note = siRNA strand - modified as per Table 2
tttggccataaggctatggtg

SEQ ID NO: 193 - RNA, 19 nt, synthetic construct
  source 1..19: mol_type = other RNA
  misc_feature 1..19: note = siRNA strand - modified as per Table 5
caggcagccaaacctcata

SEQ ID NO: 194 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 195 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 196 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 197 - RNA, 22 nt, synthetic construct
  source 1..22: mol_type = other RNA
  misc_feature 1..22: note = siRNA strand - modified as per Table 5
tatgaggttttggctgcctggg

SEQ ID NO: 198 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 199 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 200 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 201 - RNA, 22 nt, synthetic construct
  source 1..22: mol_type = other RNA
  misc_feature 1..22: note = siRNA strand - modified as per Table 5
tatgaggttttggctgcctggg

SEQ ID NO: 202 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 203 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 204 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 205 - RNA, 22 nt, synthetic construct
  source 1..22: mol_type = other RNA
  misc_feature 1..22: note = siRNA strand - modified as per Table 5
tatgaggttttggctgcctggg

SEQ ID NO: 206 - RNA, 21 nt, synthetic construct
  source 1..21: mol_type = other RNA
  misc_feature 1..21: note = siRNA strand - modified as per Table 5
tatgaggtttggctgcctggg

SEQ ID NO: 207 - RNA, 19 nt, synthetic construct
  source 1..19: mol_type = other RNA
  misc_feature 1..19: note = siRNA strand - modified as per Table 5
ctatgagcctgacctggaa

SEQ ID NO: 208 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 209 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 210 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 211 - RNA, 22 nt, synthetic construct
  source 1..22: mol_type = other RNA
  misc_feature 1..22: note = siRNA strand - modified as per Table 5
ttccaggttcaggctcatagtt

SEQ ID NO: 212 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 213 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 214 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 215 - RNA, 22 nt, synthetic construct
  source 1..22: mol_type = other RNA
  misc_feature 1..22: note = siRNA strand - modified as per Table 5
ttccaggttcaggctcatagtt

SEQ ID NO: 216 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 217 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 218 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 219 - RNA, 22 nt, synthetic construct
  source 1..22: mol_type = other RNA
  misc_feature 1..22: note = siRNA strand - modified as per Table 5
ttccaggttcaggctcatagtt

SEQ ID NO: 220 - RNA, 21 nt, synthetic construct
  source 1..21: mol_type = other RNA
  misc_feature 1..21: note = siRNA strand - modified as per Table 5
ttccaggtcaggctcatagtt

SEQ ID NO: 221 - RNA, 19 nt, synthetic construct
  source 1..19: mol_type = other RNA
  misc_feature 1..19: note = siRNA strand - modified as per Table 5
ttccttcaatcaaacccaa

SEQ ID NO: 222 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 223 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 224 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 225 - RNA, 22 nt, synthetic construct
  source 1..22: mol_type = other RNA
  misc_feature 1..22: note = siRNA strand - modified as per Table 5
ttgggttttgattgaaggaagg

SEQ ID NO: 226 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 227 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 228 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 229 - RNA, 22 nt, synthetic construct
  source 1..22: mol_type = other RNA
  misc_feature 1..22: note = siRNA strand - modified as per Table 5
ttgggttttgattgaaggaagg

SEQ ID NO: 230 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 231 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 232 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 233 - RNA, 22 nt, synthetic construct
  source 1..22: mol_type = other RNA
  misc_feature 1..22: note = siRNA strand - modified as per Table 5
ttgggttttgattgaaggaagg

SEQ ID NO: 234 - RNA, 21 nt, synthetic construct
  source 1..21: mol_type = other RNA
  misc_feature 1..21: note = siRNA strand - modified as per Table 5
ttgggtttgattgaaggaagg

SEQ ID NO: 235 - RNA, 19 nt, synthetic construct
  source 1..19: mol_type = other RNA
  misc_feature 1..19: note = siRNA strand - modified as per Table 5
agaagtctcctcttcatca

SEQ ID NO: 236 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 237 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 238 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 239 - RNA, 22 nt, synthetic construct
  source 1..22: mol_type = other RNA
  misc_feature 1..22: note = siRNA strand - modified as per Table 5
tgatgaatgaggagacttctgc

SEQ ID NO: 240 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 241 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 242 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 243 - RNA, 22 nt, synthetic construct
  source 1..22: mol_type = other RNA
  misc_feature 1..22: note = siRNA strand - modified as per Table 5
tgatgaatgaggagacttctgc

SEQ ID NO: 244 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 245 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 246 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 247 - RNA, 22 nt, synthetic construct
  source 1..22: mol_type = other RNA
  misc_feature 1..22: note = siRNA strand - modified as per Table 5
tgatgaatgaggagacttctgc

SEQ ID NO: 248 - RNA, 21 nt, synthetic construct
  source 1..21: mol_type = other RNA
  misc_feature 1..21: note = siRNA strand - modified as per Table 5
tgatgaagaggagacttctgc

SEQ ID NO: 249 - RNA, 19 nt, synthetic construct
  source 1..19: mol_type = other RNA
  misc_feature 1..19: note = siRNA strand - modified as per Table 5
cattgagagtgtcctgaaa

SEQ ID NO: 250 - RNA, 21 nt, synthetic construct
  source 1..21: mol_type = other RNA
  misc_feature 1..21: note = siRNA strand - modified as per Table 5
tttcaggacactctcaatgag

SEQ ID NO: 251 - RNA, 21 nt, synthetic construct
  source 1..21: mol_type = other RNA
  misc_feature 1..21: note = siRNA strand - modified as per Table 5
tttcaggacactctcaatgag

SEQ ID NO: 252 - RNA, 21 nt, synthetic construct
  source 1..21: mol_type = other RNA
  misc_feature 1..21: note = siRNA strand - modified as per Table 5
tttcaggacactctcaatgag

SEQ ID NO: 253 - RNA, 22 nt, synthetic construct
  source 1..22: mol_type = other RNA
  misc_feature 1..22: note = siRNA strand - modified as per Table 5
tttcaggtacactctcaatgag

SEQ ID NO: 254 - RNA, 21 nt, synthetic construct
  source 1..21: mol_type = other RNA
  misc_feature 1..21: note = siRNA strand - modified as per Table 5
tttcaggacactctcaatgag

SEQ ID NO: 255 - RNA, 21 nt, synthetic construct
  source 1..21: mol_type = other RNA
  misc_feature 1..21: note = siRNA strand - modified as per Table 5
tttcaggacactctcaatgag

SEQ ID NO: 256 - RNA, 21 nt, synthetic construct
  source 1..21: mol_type = other RNA
  misc_feature 1..21: note = siRNA strand - modified as per Table 5
tttcaggacactctcaatgag

SEQ ID NO: 257 - RNA, 22 nt, synthetic construct
  source 1..22: mol_type = other RNA
  misc_feature 1..22: note = siRNA strand - modified as per Table 5
tttcaggtacactctcaatgag

SEQ ID NO: 258 - RNA, 21 nt, synthetic construct
  source 1..21: mol_type = other RNA
  misc_feature 1..21: note = siRNA strand - modified as per Table 5
tttcaggacactctcaatgag

SEQ ID NO: 259 - RNA, 21 nt, synthetic construct
  source 1..21: mol_type = other RNA
  misc_feature 1..21: note = siRNA strand - modified as per Table 5
tttcaggacactctcaatgag

SEQ ID NO: 260 - RNA, 21 nt, synthetic construct
  source 1..21: mol_type = other RNA
  misc_feature 1..21: note = siRNA strand - modified as per Table 5
tttcaggacactctcaatgag

SEQ ID NO: 261 - RNA, 22 nt, synthetic construct
  source 1..22: mol_type = other RNA
  misc_feature 1..22: note = siRNA strand - modified as per Table 5
tttcaggtacactctcaatgag

SEQ ID NO: 262 - RNA, 21 nt, synthetic construct
  source 1..21: mol_type = other RNA
  misc_feature 1..21: note = siRNA strand - modified as per Table 5
tttcaggacactctcaatgag

SEQ ID NO: 263 - RNA, 19 nt, synthetic construct
  source 1..19: mol_type = other RNA
  misc_feature 1..19: note = siRNA strand - modified as per Table 5
aaaggaggacacttatgaa

SEQ ID NO: 264 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 265 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 266 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 267 - RNA, 22 nt, synthetic construct
  source 1..22: mol_type = other RNA
  misc_feature 1..22: note = siRNA strand - modified as per Table 5
ttcataatgtgtcctcctttgt

SEQ ID NO: 268 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 269 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 270 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 271 - RNA, 22 nt, synthetic construct
  source 1..22: mol_type = other RNA
  misc_feature 1..22: note = siRNA strand - modified as per Table 5
ttcataatgtgtcctcctttgt

SEQ ID NO: 272 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 273 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 274 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 275 - RNA, 22 nt, synthetic construct
  source 1..22: mol_type = other RNA
  misc_feature 1..22: note = siRNA strand - modified as per Table 5
ttcataatgtgtcctcctttgt

SEQ ID NO: 276 - RNA, 21 nt, synthetic construct
  source 1..21: mol_type = other RNA
  misc_feature 1..21: note = siRNA strand - modified as per Table 5
ttcataagtgtcctcctttgt

SEQ ID NO: 277 - RNA, 19 nt, synthetic construct
  source 1..19: mol_type = other RNA
  misc_feature 1..19: note = siRNA strand - modified as per Table 5
tgtacaacgtgcgcataaa

SEQ ID NO: 278 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 279 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 280 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 281 - RNA, 22 nt, synthetic construct
  source 1..22: mol_type = other RNA
  misc_feature 1..22: note = siRNA strand - modified as per Table 5
tttatgctgcacgttgtacatg

SEQ ID NO: 282 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 283 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 284 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 285 - RNA, 22 nt, synthetic construct
  source 1..22: mol_type = other RNA
  misc_feature 1..22: note = siRNA strand - modified as per Table 5
tttatgctgcacgttgtacatg

SEQ ID NO: 286 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 287 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 288 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 289 - RNA, 22 nt, synthetic construct
  source 1..22: mol_type = other RNA
  misc_feature 1..22: note = siRNA strand - modified as per Table 5
tttatgctgcacgttgtacatg

SEQ ID NO: 290 - RNA, 21 nt, synthetic construct
  source 1..21: mol_type = other RNA
  misc_feature 1..21: note = siRNA strand - modified as per Table 5
tttatgcgcacgttgtacatg

SEQ ID NO: 291 - RNA, 19 nt, synthetic construct
  source 1..19: mol_type = other RNA
  misc_feature 1..19: note = siRNA strand - modified as per Table 5
cggtcatactgtcggttaa

SEQ ID NO: 292 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 293 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 294 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 295 - RNA, 22 nt, synthetic construct
  source 1..22: mol_type = other RNA
  misc_feature 1..22: note = siRNA strand - modified as per Table 5
ttaaccgtacagtatgaccgtc

SEQ ID NO: 296 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 297 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 298 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 299 - RNA, 22 nt, synthetic construct
  source 1..22: mol_type = other RNA
  misc_feature 1..22: note = siRNA strand - modified as per Table 5
ttaaccgtacagtatgaccgtc

SEQ ID NO: 300 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 301 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 302 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 303 - RNA, 22 nt, synthetic construct
  source 1..22: mol_type = other RNA
  misc_feature 1..22: note = siRNA strand - modified as per Table 5
ttaaccgtacagtatgaccgtc

SEQ ID NO: 304 - RNA, 21 nt, synthetic construct
  source 1..21: mol_type = other RNA
  misc_feature 1..21: note = siRNA strand - modified as per Table 5
ttaaccgacagtatgaccgtc

SEQ ID NO: 305 - RNA, 2886 nt, Homo sapiens
  source 1..2886: mol_type = mRNA
atcgctgggggacagcctgcaggcttcaggaggggacacaagcatggagcggctttggggtctattccagagagcgcaacaactgtccccaagatcctctcagaccgtctaccagcgtgtggaaggcccccggaaagggcacctggaggaggaagaggaagacggggaggagggggcggagacattggcccacttctgccccatggagctgaggggccctgagcccctgggctctagacccaggcagccaaacctcattccctgggcggcagcaggacggagggctgccccctacctggtcctgacggccctgctgatcttcactggggccttcctactgggctacgtcgccttccgagggtcctgccaggcgtgcggagactctgtgttggtggtcagtgaggatgtcaactatgagcctgacctggatttccaccagggcagactctactggagcgacctccaggccatgttcctgcagttcctgggggaggggcgcctggaggacaccatcaggcaaaccagccttcgggaacgggtggcaggctcggccgggatggccgctctgactcaggacattcgcgcggcgctctcccgccagaagctggaccacgtgtggaccgacacgcactacgtggggctgcaattcccggatccggctcaccccaacaccctgcactgggtcgatgaggccgggaaggtcggagagcagctgccgctggaggaccctgacgtctactgcccctacagcgccatcggcaacgtcacgggagagctggtgtacgcccactacgggcggcccgaagacctgcaggacctgcgggccaggggcgtggatccagtgggccgcctgctgctggtgcgcgtgggggtgatcagcttcgcccagaaggtgaccaatgctcaggacttcggggctcaaggagtgctcatatacccagagccagcggacttctcccaggacccacccaagccaagcctgtccagccagcaggcagtgtatggacatgtgcacctgggaactggagacccctacacacctggcttcccttccttcaatcaaacccagttccctccagttgcatcatcaggccttcccagcatcccagcccagcccatcagtgcagacattgcctcccgcctgctgaggaagctcaaaggccctgtggccccccaagaatggcaggggagcctcctaggctccccttatcacctgggccccgggccacgactgcggctagtggtcaacaatcacaggacctccacccccatcaacaacatcttcggctgcatcgaaggccgctcagagccagatcactacgttgtcatcggggcccagagggatgcatggggcccaggagcagctaaatccgctgtggggacggctatactcctggagctggtgcggaccttttcctccatggtgagcaacggcttccggccccgcagaagtctcctcttcatcagctgggacggtggtgactttggaagcgtgggctccacggagtggctagagggctacctcagcgtgctgcacctcaaagccgtagtgtacgtgagcctggacaacgcagtgctgggggatgacaagtttcatgccaagaccagcccccttctgacaagtctcattgagagtgtcctgaagcaggtggattctcccaaccacagtgggcagactctctatgaacaggtggtgttcaccaatcccagctgggatgctgaggtgatccggcccctacccatggacagcagtgcctattccttcacggcctttgtgggagtccctgccgtcgagttctcctttatggaggacgaccaggcctacccattcctgcacacaaaggaggacacttatgagaacctgcataaggtgctgcaaggccgcctgcccgccgtggcccaggccgtggcccagctcgcagggcagctcctcatccggctcagccacgatcgcctgctgcccctcgacttcggccgctacggggacgtcgtcctcaggcacatcgggaacctcaacgagttctctggggacctcaaggcccgcgggctgaccctgcagtgggtgtactcggcgcggggggactacatccgggcggcggaaaagctgcggcaggagatctacagctcggaggagagagacgagcgactgacacgcatgtacaacgtgcgcataatgcgggtggagttctacttcctttcccagtacgtgtcgccagccgactccccgttccgccacatcttcatgggccgtggagaccacacgctgggcgccctgctggaccacctgcggctgctgcgctccaacagctccgggacccccggggccacctcctccactggcttccaggagagccgtttccggcgtcagctagccctgctcacctggacgctgcaaggggcagccaatgcgcttagcggggatgtctggaacattgataacaacttctgaggccctggggatcctcacatccccgtcccccagtcaagagctcctctgctcctcgcttgaatgattcagggtcagggaggtggctcagagtccacctctcattgctgatcaatttctcattacccctacacatctctccacggagcccagaccccagcacagatatccacacaccccagccctgcagtgtagctgaccctaatgtgacggtcatactgtcggttaatcagagagtagcatcccttcaatcacagccccttcccctttctggggtcctccatacctagagaccactctgggaggtttgctaggccctgggacctggccagctctgttagtgggagagatcgctggcaccatagccttatggccaacaggtggtctgtggtgaaaggggcgtggagtttcaatatcaataaaccacctgatatcaataa

SEQ ID NO: 306 - RNA, 19 nt, synthetic construct
  source 1..19: mol_type = other RNA
cggtcatactgtcggttaa

SEQ ID NO: 307 - RNA, 21 nt, synthetic construct
  source 1..21: mol_type = other RNA
ttaaccgacagtatgaccgtc

SEQ ID NO: 308 - RNA, 19 nt, synthetic construct
  source 1..19: mol_type = other RNA
  misc_feature 1..19: note = siRNA strand - modified as per Table 5
cggtcatactgtcggttaa

SEQ ID NO: 309 - RNA, 19 nt, synthetic construct
  source 1..19: mol_type = other RNA
  misc_feature 1..19: note = siRNA strand - modified as per Table 5
caggcagccaaacctcata

SEQ ID NO: 310 - RNA, 19 nt, synthetic construct
  source 1..19: mol_type = other RNA
  misc_feature 1..19: note = siRNA strand - modified as per Table 5
ctatgagcctgacctggaa

SEQ ID NO: 311 - RNA, 19 nt, synthetic construct
  source 1..19: mol_type = other RNA
  misc_feature 1..19: note = siRNA strand - modified as per Table 5
ttccttcaatcaaacccaa

SEQ ID NO: 312 - RNA, 19 nt, synthetic construct
  source 1..19: mol_type = other RNA
  misc_feature 1..19: note = siRNA strand - modified as per Table 5
agaagtctcctcttcatca

SEQ ID NO: 313 - RNA, 19 nt, synthetic construct
  source 1..19: mol_type = other RNA
  misc_feature 1..19: note = siRNA strand - modified as per Table 5
cattgagagtgtcctgaaa

SEQ ID NO: 314 - RNA, 19 nt, synthetic construct
  source 1..19: mol_type = other RNA
  misc_feature 1..19: note = siRNA strand - modified as per Table 5
aaaggaggacacttatgaa

SEQ ID NO: 315 - RNA, 19 nt, synthetic construct
  source 1..19: mol_type = other RNA
  misc_feature 1..19: note = siRNA strand - modified as per Table 5
tgtacaacgtgcgcataaa

SEQ ID NO: 316 - RNA, 19 nt, synthetic construct
  source 1..19: mol_type = other RNA
  misc_feature 1..19: note = siRNA strand - modified as per Table 5
cggtcatactgtcggttaa

(19) United States
(12) Patent Application Publication
(10) Pub. No.: US 2026/0092281 A1
(43) Pub. Date: Apr. 2, 2026

(54) METHODS FOR TREATMENT OF ANEMIA

(71) Applicant: Dexcel Pharma Technologies Ltd., Jerusalem (IL)

(72) Inventors: Nadav Marbach-Bar, Jerusalem (IL); Elena Feinstein, Jerusalem (IL); Noa Madar-Balakirski, Jerusalem (IL)

(73) Assignee: Dexcel Pharma Technologies Ltd., Jerusalem (IL)

(21) Appl. No.: 19/339,351

(22) Filed: Sep. 25, 2025

Related U.S. Application Data
(63) Continuation-in-part of application No. 18/898,720, filed on Sep. 27, 2024, now Pat. No. 12,454,693.

(51) Int. Cl. C12N 15/113; A61P 7/06; A61P 29/00

(52) U.S. Cl. CPC C12N 15/1138; A61P 7/06; A61P 29/00; C12N 2310/14; C12N 2310/315; C12N 2310/321; C12N 2310/322; C12N 2310/351

(57) ABSTRACT

Provided herein are nucleic acids and compositions thereof for inhibiting expression of transferrin receptor 2 (TFR2). Also described are methods for treatment of TFR2-associated diseases, such as anemia, with the disclosed nucleic acids.

### CROSS REFERENCE TO RELATED APPLICATIONS

This is a Continuation-in-Part of U.S. patent application Ser. No. 18/898,720, filed Sep. 27, 2024, the contents of which are incorporated by reference herein in their entirety.

### BRIEF DESCRIPTION OF THE SEQUENCE LISTING

The nucleotide sequences provided herewith are shown using standard letter abbreviations for nucleotide bases as defined in 37 CFR 1.831 through 37 CFR 1.835. Only one strand of each nucleic acid sequence is shown, but the complementary strand is understood as included by any reference to the displayed strand. The Sequence Listing is submitted as an XML file named 3316_1_seqlist, approximately 399,000 bytes, created Sep. 21, 2025, the contents of which are incorporated by reference herein in their entirety.

### FIELD

This disclosure relates to nucleic acids and compositions thereof for inhibiting expression of transferrin receptor 2 (TFR2). Also described are methods for treatment of TFR2-associated diseases, such as anemia, with the disclosed nucleic acids.

### BACKGROUND

The hepcidin antimicrobial peptide (encoded primarily in hepatocytes by the HAMP gene) is a significant modulator of systemic iron homeostasis (Liu et al., Medicine, Volume 95, Number 14, April 2016). Its expression is tightly controlled by various factors, including serum and tissue iron levels, inflammation, and hypoxia. TFR2, a transmembrane protein predominantly expressed in hepatocytes and erythroid precursors, plays a pivotal role in iron sensing and signaling and, by extension, in regulation of erythropoiesis. In conditions of iron overload, TFR2 interacts with iron-bound transferrin, leading to downstream signaling events and induction of hepcidin expression. TFR2-deficient mice show reduced levels of phosphorylated SMAD1/5/8 in the liver, suggesting that TFR2 regulates hepcidin via the BMP-SMAD pathway. Additionally, TFR2-deficient mice have blunted hepcidin response to inflammation suggesting a crosstalk with IL6-STAT-mediated regulation of hepcidin expression. The function of TFR2 in hepcidin and iron regulation has been validated by human genetics. Humans with loss of function mutations in the TFR2 gene present with a condition of iron overload, called hemochromatosis, characterized by excessive iron absorption and reduced hepcidin (Wang et al., Blood 133:18-29, 2019; Wallace et al., Blood 117:2960-2966, 2011). Therefore, while HAMP overexpression is associated with iron deficiency anemia, HAMP underexpression can result in iron overload diseases such as hereditary hemochromatosis. Reducing HAMP overexpression could therefore provide targeted treatment for HAMP-related anemias. Such reduction of hepcidin levels can be achieved directly, e.g., via antisense oligonucleotide or siRNA targeting of the HAMP gene, or indirectly by targeting genetic elements of the upstream pathways that regulate hepcidin levels, such as TFR2.

Previous efforts at directly reducing TFR2 using siRNA have been described (e.g., in U.S. Pat. No. 9,228,188 and WO 2023/176,862). However, these reported efforts have not developed into clinical-stage therapeutics. Thus, a continuing need exists for siRNA molecules to robustly and reliably reduce the expression and associated activity of TFR2 and by extension pathological amounts of hepcidin in circulation.

### SUMMARY

The present disclosure describes the discovery of novel, highly active, specific, and metabolically stable siRNAs for inhibiting expression of TFR2 mRNA. In particular, it was observed that particular siRNAs of a particular sequence and possessing unique chemical modification patterns can robustly and specifically knock down TFR2 mRNA expression, both in vitro and in vivo, while displaying superb metabolic stability, which can provide therapeutically relevant knock down of TFR2 mRNA in vivo without the need for a transcription facilitating or mediating reagent, such as a lipid-based transfection agent. The described siRNAs are additionally predicted not to exhibit sequence-dependent off-target knockdown activity with their potential seed-mediated off-target activity being efficiently neutralized. The extensive assays described herein show the discovery and optimization of the described compounds, and additionally demonstrate that the ability to effectively inhibit expression of TFR2 mRNA is sensitive to the region of TFR2 mRNA targeted, and variations in oligonucleotide chemical modifications, with variations affecting overall activity and/or specificity. Accordingly, the presently described siRNAs provide novel therapeutic agents which are advantageously active, specific, and safe.

Described herein are double-stranded nucleic acids for inhibiting expression of transferrin receptor 2 (TFR2), that include a sense strand including a sense strand sequence and an antisense strand including an antisense strand sequence, in which the sense strand sequence includes the sequence 5′ CGGUCAUACUGUCGGUUAA 3′ (SEQ ID NO: 306) and/or the antisense strand sequence includes the sequence 5′ UUAACCGACAGUAUGACCGUC 3′ (SEQ ID NO: 307), which can in particular embodiments be partially or fully modified, and in more particular embodiments can include 1, 2, 3, 4, 5, or more additions, deletions or substitutions in the sense and/or the antisense strand sequence.

In particular embodiments, the sense strand includes the sense strand sequence 5′-(C3)csgsguCfaUfAfCfugucgguuaa 3′ (SEQ ID NO: 308), in which a lowercase letter indicates a 2′-O-Methyl (2′-OMe) modified RNA nucleotide, an uppercase letter followed by f indicates a 2′-Fluoro (2′-F) modified RNA nucleotide, (C3) indicates a propanol or C3 alkyl moiety connected to the 5′ end of the strand via a phosphodiester linkage, and s indicates a phosphorothioate linkage between adjacent nucleotides.

In particular embodiments of the described double-stranded nucleic acids, the sense strand sequence includes 5′-(C3)csgsguCfaUfAfCfugucgguuaasLsLsL-3 (SEQ ID NO: 291), in which L indicates a monomer of N-acetylgalactosamine (GalNAc) with a triethyleneglycol (TEG) linker, and which connected via a phosphorothioate linkage as noted.

In other particular embodiments, the antisense strand includes an antisense strand sequence 5′-(vinu)sUfsaacCf(3dG)AfCfaguaUfgAfccgsusc-3′ (SEQ ID NO: 293) or 5′-(vinu)sUfsaacCf(3dG)aCfaguaUfgAfccgsusc-3′ (SEQ ID NO: 297), in which (vinu) indicates a 5′ vinylphosphonate 2′-OMe RNA U nucleotide, and (3dG) indicates a 3′H modification of ribose in a G nucleotide that is connected via a 2′-5′ bridge with the following nucleotide.

In some embodiments of the described double-stranded nucleic acids, the sense strand sequence includes 5′-(C3)csgsguCfaUfAfCfugucgguuaasLsLsL-3 (SEQ ID NO: 291), in which L indicates a monomer of N-acetylgalactosamine (GalNAc) with a triethyleneglycol (TEG) linker, which is shown to be connected via a phosphorothioate linkage, and the antisense strand sequence includes 5′-(vinu)sUfsaacCf(3dG)AfCfaguaUfgAfccgsusc-3′ (SEQ ID NO: 293) or 5′-(vinu)sUfsaacCf(3dG)aCfaguaUfgAfccgsusc-3′ (SEQ ID NO: 297), in which (vinu) indicates a 5′ vinylphosphonate 2′-OMe RNA U nucleotide, and (3dG) indicates a 3′H modification of ribose in a G nucleotide that is connected via a 2′-5′ bridge with the following nucleotide.

Particular embodiments of the described double-stranded nucleic acids further include a targeting ligand that is covalently attached to the double-stranded nucleic acid, such as at least one of the 5′ end of the sense strand, the 3′ end of the sense strand, or the 3′ end of the antisense strand, or any non-terminal nucleotide along the sense or antisense strands, and wherein the 3′ ends of either the sense and/or of the antisense strands, if not attached to a targeting ligand, are extended to have a 3′ overhang.

In some embodiments of the described double-stranded nucleic acids which include a targeting ligand, the targeting ligand is selected from one or more moieties that include but are not limited to a carbohydrate, lipophilic, any type of antibody, polypeptide, peptide, peptidomimetic, aptamer, vitamins, or any type of small molecule moieties that support cell targeting via binding to cellular membrane structures followed by siRNA internalization. In particular embodiments, the carbohydrate moiety is 1, 2, 3, or 4 N-acetylgalactosamine (GalNAc) moieties, which in still further particular embodiments is or are covalently attached to the 3′ end of the sense strand, and even more particularly in certain embodiments can be linked to the sense strand via a triethyleneglycol linker.

In particular embodiments, the described double-stranded nucleic acids are for use in medicine, for such as but not limited to for use in treatment of an anemia and particularly a HAMP-related anemia as defined herein.

Also described herein is a pharmaceutical composition that includes the described double-stranded nucleic acid or a pharmaceutically acceptable salt thereof, and one or more of a pharmaceutically acceptable excipient, carrier, or buffer.

In particular embodiments of the pharmaceutical composition, the double-stranded nucleic acid is the only pharmaceutically active agent.

In other particular embodiments, the described pharmaceutical composition is for use in treatment of an anemia, such as particularly a HAMP-related anemia

Further described herein is a method for treating an anemia, such as a HAMP-related anemia by administering to a subject in need thereof, an effective amount of the described pharmaceutical composition, thereby treating the anemia.

The foregoing and other objects, features, and advantages will become more apparent from the following detailed description, which proceeds with reference to the accompanying figures.

As described above and further listed below, the nucleic acid molecules described herein include nucleotide and extranucleotide (e.g., backbone and non-nucleotide moiety) modifications. The naming conventions of the noted modifications in the described sequences is shown in below Table 1. The listed modifications are further described herein.

TABLE 1
Sequence Naming Conventions

| Symbol | Modification Type |
|---|---|
| N | non-modified |
| | ribonucleotide |
| n | A nucleotide with 2′-OMe |
| | modification of ribose |
| Nf | A nucleotide with 2′-F |
| | modification of ribose |
| 3dN | 3′H modification of |
| | ribose in a nucleotide N |
| | that is connected via 2′-5′ |
| | bridge with the next one. |
| (2-5n) | 3′OMe modification of |
| | ribose in a nucleotide |
| | that is connected via 2′-5′ |
| | bridge with the next one. |
| Nt | A nucleotide having |
| | threose instead of ribose |
| | as a sugar moiety (TNA) |
| vinu | −5′-E-vinylphosphonate |
| | 2′OMe U |
| s | Phosphorothioate |
| | backbone modification |
| | between 2 adjacent |
| | nucleotides |
| L | Monomer of N- |
| | acetylgalactosamine |
| | (GalNAc) with a |
| | triethyleneglycol (TEG) |
| | linker connected via a |
| | phosphodiester linkage |
| | (or phosphorothioate if |
| | noted with an “s”) |
| C3 | propanol or C3 alkyl |
| | moiety connected via a |
| | phosphodiester linkage |

TABLE 1A
TFR2 targeting sequences

| SEQ ID | Starting | | Sense strand |
|---|---|---|---|
| NO | nt | siRNA ID | Antisense strand |
| 291 | 2657 | XD-65806 | 5′-(C3)csgsguCfaUfAfCfugucgguuaasLsLsL-3′ |
| 292 | | | 5′-(vinu)sUfsaacCfgAfCfaguaUfgAfccgsusc-3′ |
| 291 | 2657 | XD-65807 | 5′-(C3)csgsguCfaUfAfCfugucgguuaasLsLsL-3′ |
| 293 | | | 5′-(vinu)sUfsaacCf(3dG)AfCfaguaUfgAfccgsusc-3′ |
| 291 | 2657 | XD-65808 | 5′-(C3)csgsguCfaUfAfCfugucgguuaasLsLsL-3′ |
| 294 | | | 5′-(vinu)sUfsaacCf(2-5g)AfCfaguaUfgAfccgsusc-3′ |
| 291 | 2657 | XD-65809 | 5′-(C3)csgsguCfaUfAfCfugucgguuaasLsLsL-3′ |
| 295 | | | 5′-(vinu)sUfsaacCfGtAfCfaguaUfgAfccgsusc-3′ |
| 291 | 2657 | XD-65810 | 5′-(C3)csgsguCfaUfAfCfugucgguuaasLsLsL-3′ |
| 296 | | | 5′-(vinu)sUfsaacCfgaCfaguaUfgAfccgsusc-3′ |
| 291 | 2657 | XD-65811 | 5′-(C3)csgsguCfaUfAfCfugucgguuaasLsLsL-3′ |
| 297 | | | 5′-(vinu)sUfsaacCf(3dG)aCfaguaUfgAfccgsusc-3′ |
| 291 | 2657 | XD-65812 | 5′-(C3)csgsguCfaUfAfCfugucgguuaasLsLsL-3′ |
| 298 | | | 5′-(vinu)sUfsaacCf(2-5g)aCfaguaUfgAfccgsusc-3′ |
| 291 | 2657 | XD-65813 | 5′-(C3)csgsguCfaUfAfCfugucgguuaasLsLsL-3′ |
| 299 | | | 5′-(vinu)sUfsaacCfGtaCfaguaUfgAfccgsusc-3′ |
| 291 | 2657 | XD-65814 | 5′-(C3)csgsguCfaUfAfCfugucgguuaasLsLsL-3′ |
| 300 | | | 5′-(vinu)sUfsaaccgacaguaUfgAfccgsusc-3′ |
| 291 | 2657 | XD-65815 | 5′-(C3)csgsguCfaUfAfCfugucgguuaasLsLsL-3′ |
| 301 | | | 5′-(vinu)sUfsaacc(3dG)acaguaUfgAfccgsusc-3′ |
| 291 | 2657 | XD-65816 | 5′-(C3)csgsguCfaUfAfCfugucgguuaasLsLsL-3′ |
| 302 | | | 5′-(vinu)sUfsaacc(2-5g)acaguaUfgAfccgsusc-3′ |
| 291 | 2657 | XD-65817 | 5′-(C3)csgsguCfaUfAfCfugucgguuaasLsLsL-3′ |
| 303 | | | 5′-(vinu)sUfsaaccGtacaguaUfgAfccgsusc-3′ |
| 316 | 2657 | XD-65818 | 5′-(C3)csgsGfucaUfAfCfugUfcgguUfaasLsLsL-3′ |
| 304 | | | 5′-(vinu)sUfsaaccgacaguaUfgaccgsusc-3′ |
| 308 | 2657 | | (C3)csgsguCfaUfAfCfugucgguuaa (sense strand only) |

### BRIEF DESCRIPTION OF THE FIGURES

FIG. 1 shows the dose-dependent activity of chemically optimized TFR2 siRNAs in primary human hepatocytes (PHH). The siRNAs were delivered via free uptake mediated by a GalNAc ligands. The graphs show normalized residual expression levels (%) of human TFR2 mRNA in PHH treated with indicated siRNAs at a dose range from 1.8 mM to 6 pM in 3-fold dilution steps. % values were all normalized to the mean relative quantity (Rq) of control non-treated cells to obtain the mean relative abundance as a percentage of expression in non-treated cells. Error bars represent standard deviations.

FIG. 2A shows knockdown of human TFR2 mRNA levels in the livers of PXB mice on Day 15 after treatment. Residual expression levels (%) of human TFR2 mRNA in the livers of PXB mice treated with different doses—0 (PBS), 5, 10, or 30 mg/kg, of siRNAs XD-65807, XD-65811 or XD-65815 on Day 15 after study drug administration are shown. All % values are adjusted to standard curve and normalized to mean of TFR2 RNA levels in PBS control. Each data point represents the mean percentage of residual human TFR2 expression across the three liver lobes analyzed for each animal. Error bars are standard deviations. Statistical analysis was done using the one-way ANOVA followed by Dunnett's multiple comparison test with significance set for p<0.05 (GraphPad Prism package). Each treatment group was compared to the PBS control group. ***=p<0.0001.

FIG. 2B shows knockdown of human TFR2 mRNA levels in the livers of PXB mice on Day 49 after treatment. Residual expression levels (%) of human TFR2 mRNA in the livers of PXB mice treated with different doses—0 (PBS) or 10 mg/kg, of siRNAs XD-65807, XD-65811 or XD-65815 on Day 49 after study drug administration are shown. All % values are adjusted to standard curve and normalized to mean of TFR2 RNA levels in PBS control. Each data point represents the mean percentage of residual human TFR2 expression across the three liver lobes analyzed for each animal. Error bars are standard deviations. Statistical analysis was done using the one-way ANOVA followed by Dunnett's multiple comparison test with statistical significance set for p<0.05 (GraphPad Prism package). Each treatment group was compared to the PBS control group. **=p<0.01 and *=p<0.05. The comparison between the XD-65815 group and the PBS control group was hampered due to having only one mouse in the XD-65815 group (n=1).

FIG. 3 shows time dependent changes in serum hepcidin (A) and iron (B) levels elicited by acute intravenous administration of IL-6 to cynomolgus monkeys (Day −14).

FIG. 4 shows TFR2 knockdown (percentage of non-treated control) in liver biopsies of monkeys 6 weeks after TFR2 siRNA treatment. Data are presented as mean±SEM (N=3/group). Statistical significance versus non-treated control was determined using Dunnett's multiple comparison test (**** p<0.0001). Real-time RT-PCR.

FIG. 5 shows the effect of TFR2 siRNA treatment on acute inflammation (IL-6 treatment) induced changes in serum hepcidin (A) and iron levels (B). Data are presented as mean±SEM (N=3/group). Statistical significance versus pre-treatment control was determined using Dunnett's multiple comparison test (**** p<0.0001, ** p<0.01, * p<0.05).

FIG. 6 shows dose- and time-dependent changes in TFR2 mRNA knockdown (percentage of non-treated control) in the livers of cynomolgus monkeys following TFR2 siRNA administration and exposure to the model of acute inflammation. Data are presented as mean±SEM (N=3/group). Statistical significance versus non-treated control was determined using Dunnett's multiple comparison test (**** p<0.0001, ** p<0.01). Real-time RT-PCR.

FIGS. 7A-7C show TFR2 siRNA dose- and time-dependent changes in serum hepcidin (FIG. 7A), serum iron (FIG. 7B) and transferrin saturation (TSAT, FIG. 7C) levels in cynomolgus monkeys following exposure to the model of acute inflammation. Data are presented as mean±SEM (N=3/group).

FIGS. 8A-8D show dose- and time-dependent TFR2 siRNA-induced changes in erythropoiesis in cynomolgus monkeys after exposure to the model of acute inflammation: reticulocyte hemoglobin (RHE, FIG. 8A), mean corpuscular hemoglobin (MCH, FIG. 8B), hematocrit (HCT, FIG. 8C), and total hemoglobin (Hb, FIG. 8D) levels. Data are normalized to individual baseline values and presented as mean±SEM (N=3/group).

FIG. 9 shows dose- and time-dependent TFR2 mRNA knockdown in the livers of normal cynomolgus monkeys following two TFR2 siRNA administrations. Data are presented as mean±SD (N=3/group). Statistical significance versus pre-treatment control was determined using Dunnett's multiple comparison test (**** p<0.0001, *** p<0.001, ** p<0.01, * p<0.05).

FIGS. 10A and 10B show dose- and time-dependent dependent changes in serum iron (FIG. 10A) and transferrin saturation (TSAT, FIG. 10B) levels following two TFR2 siRNA administrations in normal cynomolgus monkeys. Data are presented as mean±SD (N=2-3/group).

### DETAILED DESCRIPTION

### Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes, and all molecular weight or molecular mass values, given for nucleic acids are approximate, and are provided for description. Although methods and materials similar or equivalent to those described herein can be used in the practice or testing of this disclosure, suitable methods and materials are described below. The term “comprises” means “includes.” The term “consists essentially of” or “consisting essentially of” indicates that the active ingredient or step of the described composition or method includes only the expressly recited ingredient or step, but that non-active ingredients or non-critical steps are included even if not expressly recited. This is in contrast to “consists” or “consisting” of which indicates that only the recited ingredients or steps are included in the claim. It is to be understood that compositions that “comprise” a given ingredient can also in other embodiments “consist essentially of” or “consist of” that ingredient. Similarly, methods that “comprise” a given set of steps can also in other embodiments “consist essentially of” or “consist of” the expressly indicated set of steps. The abbreviation, “e.g.,” is derived from the Latin exempli gratia, and is used herein to indicate a non-limiting example. Thus, the abbreviation “e.g.,” is synonymous with the term “for example.”

In case of conflict, the present specification, including explanations of terms, will control. All materials, methods, and examples are illustrative and not intended to be limiting. References cited herein are incorporated by reference herein with respect to the technical aspects described (chemical structure, sequence modifications, etc.).

### Nucleic Acids for Inhibiting TFR2 Expression

Described herein are synthetic nucleic acid molecules for reducing the amount of TFR2 mRNA, and by extension inhibiting the expression of the hepcidin peptide that is dependent on expression of TFR2 for HAMP mRNA (encoding hepcidin peptide) expression, in a cell or organism that expresses HAMP. The described nucleic acid molecules, also referred to as nucleic acid “compounds” or nucleic acid “compositions” are suitable for RNA interference or inhibition of expression of the TFR2 target gene. As used herein, the term “mRNA knockdown,” is understood to be synonymous with the concept of post-transcriptional inhibition of TFR2 expression (see Zhu et al., Cell Death Dis. 13(7):644 (2022)). As used herein, a “target” gene is a gene which the described dsRNA compounds are designed to specifically inhibit expression of by reducing the presence of the RNA encoded by the target gene. The described TFR2-targeting nucleic acids can be used, inter alia, for treatment of diseases and pathological conditions accompanied with iron deficiency associated with pathological HAMP overexpression and/or hepcidin accumulation in blood. Such “HAMP-related anemia” or alternatively “HAMP-associated anemia” include but are not limited to, hereditary iron refractory iron deficiency anemia (IRIDA), anemia associated with acute and chronic inflammatory diseases, cancer (including myelofibrosis), or chronic kidney disease (e.g., as described by Wang & Babitt, Curr. Opin. Hematol. 23(3), 189-197 (2016), Verstovsek, Annals of Hem. 102:698-698 (2023); Portoles et al. Front Med. 26; 8:642296 (2021); and Rivera et al. Blood 112(11), 3835 (2008)).

The molecules described herein can be presented as a double stranded (ds) molecule or as a single stranded (ss) molecule that is designed to form intramolecular double stranded regions which are then effectively equivalent to a double stranded molecule. Accordingly, the described molecules can be formed from ssRNAs that can hybridize one to another thereby forming a dsRNA duplex or from a single RNA molecule of sufficient internal complementarity to enable stem and loop (i.e., hairpin) formation. Antisense oligonucleotides, including but not limited to gapmers, with sufficient homology to the antisense strand sequences listed herein are also within the current disclosure. In particular embodiments, the described molecules are dsRNA, such as small interfering RNA (siRNA).

The double stranded nucleic acid molecules described herein have two polynucleotide strands (or their equivalents in the case of a ssRNA molecule with sufficient internal complementarity to form a hairpin structure): a strand with a nucleotide sequence reasonably identical to the targeted mRNA, most commonly referred to as the sense, or passenger, strand; and a reverse complementary strand also referred to as the antisense, or guide, strand. The sense and antisense strands are correspondingly composed of a sense strand nucleotide sequence (referred to herein as a “sense strand sequence”) and an antisense strand nucleotide sequence (referred to herein as an “antisense strand sequence”). In the described molecules, the antisense strand is composed of a sequence that is substantially reverse complementary to a 15-35, particularly a 17-25, and more particularly a 15, 16, 17, 18, 19, 20, 21, 22, 23, 24, 25, 26, 27, 28, 29, 30, 31, 32, 33, 34, or 35 contiguous nucleotide sequence of the human TFR2 mRNA sequence found at in the NCBI database under accession number NM_001206855.3 (SEQ ID NO: 305; available online at ncbi.nlm.nih.gov/nuccore/NM_001206855.3).

In particular embodiments, the sense strand sequence of the described molecules is fully complementary to the antisense strand sequence within the double-stranded region (excluding a 3′ overhang). In other particular embodiments, at least one of the sense or antisense strands of the described molecules is a sense or antisense strand sequence listed in Tables 2 and 5 below. In other particular embodiments, the described molecules comprise one of the sense and antisense strand sequence pairs listed in Tables 2 and 5 below.

Particular embodiments of the double stranded nucleic acid molecules described herein comprise a sense strand starting (5′) from position 2657 of SEQ ID NO:305 (referred to herein as “TFR2-2657 siRNAs”). In such embodiments, the sense strand sequence can comprise the sequence 5′ CGGUCAUACUGUCGGUUAA 3′ (SEQ ID NO: 306) and/or the antisense strand sequence can comprise the sequence 5′ UUAACCGACAGUAUGACCGUC 3′ (SEQ ID NO: 307). In particular embodiments, the described dsRNA includes at least one nucleotide or non-nucleotide (e.g., non-nucleotide ligand or moiety) addition, deletion, substitution, and/or modification to SEQ ID NOs 306 and/or 307. The described dsRNA can be partially or more preferably fully modified, such that each nucleotide is chemically modified from the unmodified sequences. In particular embodiments, the described dsRNAs can include 1, 2, 3, 4, 5, or more additions, deletions or substitutions, and in more particular embodiments no more than 1, no more than 2, no more than 3, no more than 3, no more than 4, or no more than 5 additions, deletion, or substitutions.

Particular modifications of TFR2-2657 siRNA sense and antisense sequences are additionally described below in Tables 2 and 5, according to the nucleotide and modification notation in Table 1. In particular embodiments, the described TFR2-2657 siRNA includes a sense strand sequence described herein as SEQ ID NO: 308 (5′-(C3)csgsguCfaUfAfCfugucgguuaa 3′ (see Table 1 below for notation). In other embodiments, the siRNA includes a sense strand sequence described herein as SEQ ID NO: 291, as listed in Table 5. In particular embodiments, the siRNA includes an antisense strand sequence described herein as SEQ ID NO: 293 or 297, both of which are listed in Table 5.

In view of their double stranded nature, each strand of the described nucleic acids is substantially complementary to the other, and the nucleotide sequence of one strand can be understood from the sequence of the other. Strands being complementary to each other over a certain length means that the strands are able to form a stable non-interrupted duplex at physiological conditions of at least 12 consecutive hybridized base pairs over that length, such as but not limited to beginning from the 5′ nucleotide of the antisense strand sequence. As used herein, a molecule that comprises sense and antisense strands that are “substantially complementary” means that every nucleotide of the length of the molecule does not necessarily have to be able to base pair with its counterpart in the other strand, as long as a stable double-stranded nucleic acid under physiological conditions can be formed. Nucleic acids that are capable of hybridizing under physiological conditions are nucleic acids that are capable of forming base pairs, particularly Watson-Crick or wobble base-pairs, between at least a portion of the opposed nucleotides in the strands so as to form a duplex region. Such a double-stranded nucleic acid is a stable double-stranded nucleic acid under physiological conditions, meaning that under such conditions, the two strands remain hybridized to each other.

In particular embodiments, the described double stranded nucleic acids, which in particular embodiments is a dsRNA, and more particularly an siRNA, comprises an antisense strand sequence that is 100% complementary to its target sequence. In other embodiments, the antisense strand sequence of the siRNA is only partially complementary, and has 1, 2, 3, or more nucleotides that are different from the complement of its target RNA sequence. Similarly, in particular embodiments, the strands of the dsRNA described herein can be fully complementary to each other. In other embodiments, the strands of the dsRNA can be partially complementary to each other, and can include base mismatches of 1, 2, 3, or more (sometimes referred to in the art as a “bungle”), so long as the dsRNA remains double stranded under physiological conditions. In some embodiments, variations in the sense or antisense strand sequences represent differences, in the sense strand, from the target sequence. In other embodiments, the variations in the sense and antisense strands can include a 1, 2, 3 or greater base overhang at the 3′ end, which can independently be on the sense and/or the antisense strands. For example, in certain embodiments, a two-base 3′ overhang is present on the antisense strand sequence. In more specific embodiments, the sense strand and the antisense strand each further comprises a 2-nt 3′-overhang. Overhang nucleotides can be either directly derived from the target RNA sequence (if on the sense strand) or be complementary to the target RNA sequence (if on the antisense strand) or substituted by “universal” nucleotides, for example, but not limited to dTdT or uu or ug (where “dT” is deoxythymidine, “u” is 2′OMe uridine, g is 2′OMe guanine connected between them and to the 3′ end(s) of siRNA strands via phosphodiester or phosphorothioate linkages). In particular embodiments, 3′ overhangs may be substituted by non-nucleotide moieties, such as but not limited to C3/C4-Pi/Ps-C3/C4-Pi/Ps where C3 and C4 are tri- or tetracarbon linkers connected between them and to the 3′ end(s) of siRNA strands via phosphodiester (Pi) or phosphorothioate (Ps) linkages. Particular examples of 2-nt 3′ overhangs are described herein, and include, but are not limited, to a 2′Ome uracil nucleotide followed by a 2′Ome cytosine nucleotide, joined at the 3′ end of the antisense strand sequence, and to each other, by phosphorothioate linkages.

In particular embodiments of the described double stranded nucleic acids, such as a dsRNA, the sequence of the dsRNA is defined by reference to the sense strand sequence. In other embodiments, the sequence of dsRNA is defined by reference to the antisense strand sequence. In still other embodiments, the sequence of the dsRNA is defined in reference to both the sense strand sequence and the antisense strand sequence.

In the present disclosure, distinction is made between the sense and antisense strands and the sense and antisense strand sequences. Accordingly, the described sense and antisense strands can comprise sense and antisense strand sequences that consist of particular defined nucleotide sequences which can, in particular embodiments be unmodified, partially modified (i.e., wherein not all of the nucleotides are chemically modified), or completely modified (wherein all of the nucleotides of the sequence are chemically modified). In such molecules, the sense and antisense strand sequences are expressly defined, but the sense and antisense strands as independent molecules remain open to undefined further addition or modification.

### Nucleic Acid Modifications

The double stranded nucleic acids described herein, such as dsRNA, and more specifically siRNA, can include sense and/or antisense strand sequences that are composed of unmodified nucleotides, combine nucleotides that are unmodified with those that are chemically modified, such that at least one nucleotide of one strand of the double stranded nucleic acid must be modified, or in particular embodiments are completely (i.e., “fully”) modified such that all of the nucleotides of both sense and antisense strand sequences are modified.

Unmodified double stranded nucleic acids refer to a molecule in which the components of the nucleic acid, namely sugars, bases, phosphate moieties, and internucleoside linkages, are the same or essentially the same as those which occur in nature. An unmodified (also described as a “non-modified”) nucleotide of the described nucleic acid molecules is one observed in cellular nucleic acids and is composed of a nitrogenous base [Adenine—A, Guanine—G, Thymine—T (in DNA), Cytosine—C, Uracil—U (in RNA)], a D-deoxyribose or D-ribose sugar moiety (furanose) in deoxy-ribonucleotides and ribonucleotides, respectively, and at least one phosphate group, which may make up an internucleoside linkage (backbone). An unmodified nucleotide may refer to an unmodified ribonucleotide or an unmodified deoxyribonucleotide. An unmodified nucleotide in a nucleic acid molecule is covalently linked to neighboring nucleotides by phosphodiester bonds between the sugar moieties of the nucleotides via 3′-5′ linkages, in which the 3′ position of the nucleotide's sugar moiety is linked through a phosphate with the 5′ position of the next nucleotide's sugar moiety, and the 5′ position of the nucleotide's sugar moiety is similarly linked through a phosphate with the 3′ position of the previous nucleotide's sugar moiety (in this context, “next” and “previous” relate to the 5′>3′ direction, as commonly referred in the art).

In contrast, a modified nucleotide of the described nucleic acid molecules is an unmodified nucleotide in which at least one of the nitrogenous base, the sugar, or the internucleoside linkage are independently chemically modified whereas some of these modifications may also occur in nature. It will accordingly be understood that deoxyribose nucleotides are considered to be modified ribonucleotides in the context of RNA compounds, such as, the described siRNA compounds. Similarly, a nucleotide containing uracil would be considered to be a modified nucleotide in the context of DNA compounds. In addition, the described double stranded ribonucleic acids may be modified by substitution with, or insertion of, analogs of nucleic acids or bases. Other particular non-limiting examples of modified nucleotides include naturally occurring but non-abundant nucleotide variants, such as pseudo-U or inosine (I). In other particular examples, the entire nucleotide (all its parts) is modified, such as in the case of mirror nucleotides including L-nucleotides. Additional modifications to the nucleic acids described can include but are not limited to end modifications, e.g., 5′-end modifications (phosphorylation, conjugation, inverted linkages) or 3′-end modifications (conjugated ligands, DNA nucleotides, inverted linkages); base modifications as described, and removal of bases (abasic nucleotides). Particular non-limiting examples of nucleic acid and nucleotide modifications for use with the described nucleic acids are described below (see also in Hu et al., Nature: Signal Transduct. and Targeted Ther. 5, Article 101, 2020, and International Patent Publication Nos. WO2024013334A1 and WO2023215805A1 the contents of which are incorporated by reference herein with respect to descriptions of modified nucleic acids and formulations thereof).

### Nucleotide Modifications: Sugar Moiety

In particular embodiments of the described composition, a modified nucleotide can be one in which the ribose or deoxyribose sugar moiety is chemically modified. According to some embodiments, the modification in the ribose sugar moiety is in the 2′-position not usually used in the native 5′ to 3′-internucleoside linkage.

Modifications to the sugar moiety of the described nucleic acids can include but are note limited to one of the following at the 2′-position: OH; F; O-, S-, or N-alkyl; O-, S-, or N-alkenyl; 0-, S- or N-alkynyl; or O-alky I-O-alkyl. wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are independently from 1 to about 10. In other embodiments, dsRNAs can include one of the following at the 2′ position: C₁ to C₁₀ alkyl, substituted alkyl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of the described dsRNA, or a group for improving the pharmacodynamic properties of the described dsRNA.

In particular embodiments of the described nucleic acids, chemical modification of the ribose sugar includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxy ethyl or 2′-DMAEOE), i.e., 2′-O—CH₂—O—CH₂—N(CH₃)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxy alkyl; and 2′-NMA (N-methylacetamide).

In other particular embodiments of the described dsRNAs, chemical modifications of the ribose sugar include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions, particularly the 3′ position of the sugar on the 3′ terminal nucleotide or in 2′-5′ linked dsRNAs and the 5′ position of 5′ terminal nucleotide. In still other embodiments, dsRNAs can also have sugar mimetics such as cyclobutyl moieties in place of the pentofuranosyl sugar.

The sugar moiety of the described dsRNAs also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system, such as the 4′-carbon and the 2′-carbon of the sugar ring. In particular examples, the described dsRNAs can include one or more locked nucleic acids (LNA) in which the ribose is locked in the 3′-endo structural conformation. A locked nucleic acid is a nucleotide having a modified ribose moiety which comprises an extra bridge connecting the 2′ and 4′ carbons (i.e., comprising a bicyclic sugar moiety comprising a 4′-CH2-O-2′ bridge). Particular examples of bicyclic nucleosides for use in the described dsRNAs include but are not limited to 4′ to 2′ bridged bicyclic nucleosides, such as 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′; 4′-C(CH₃)(CH₃)—O-2′; 4′-CH₂—N(OCH₃)-2′; 4′-CH₂—O—N(CH₃)-2′; 4′-CH₂—N(R)—O-2′, wherein R is H, C1-C12 alkyl, or a protecting group; 4′-CH₂—C(H)(CH₃)-2′; and 4′-CH₂—C(═CH₂)-2′.

Other particular examples of chemical modifications at the 2′ hydroxyl group of the ribose sugar include O-AMINE modifications, wherein AMINE=NH2. Such modifications include but are not limited to alkylamino, dialkylamino, heterocyclyl, arylamino, diaryl amino, heteroaryl amino, or diheteroaryl amino, ethylene diamine or polyamino) and aminoalkoxy, O(CH2)nAMINE, (e.g., AMINE=NH2), alkylamino, dialkylamino, heterocyclyl, arylamino, diaryl amino, heteroaryl amino, or diheteroaryl amino, ethylene diamine or polyamino).

Other possible modifications of the sugar moiety include substitution of the natural sugar group with a different sugar moiety, e.g. threose to yield threose nucleic acid (TNA). Modifications to the sugar group can be such that it contains one or more carbons that possess the opposite stereochemical configuration than that of the corresponding carbon in ribose. Thus, a modified nucleotide may contain a sugar such as arabinose to yield arabinonucleic acids (ANA) with or without sugar modifications like, e.g. in case of 2′-deoxy-2′-fluoroarabinonucleic acid (FANA). In other embodiments, sugar moieties may be substituted with non-sugar moieties such as e.g., propylene glycol to yield glycol nucleic acids (GNA) or with methylenemorpholine rings to yield morpholino oligonucleotides or with cyclopentyl. In yet another embodiment, ribose can be presented by its acyclic derivative lacking the 2′-3′ bond of the ring to yield unlocked nucleic acid (UNA).

In particular embodiments, the described double stranded nucleic acids include modified nucleotides with a modified sugar moiety having a 2′ carbon in the sugar moiety modified with a methyl moiety and/or a fluoro moiety, resulting in a nucleic acid that includes one or multiple 2′ O methyl (2′O-Me) modified nucleotides and/or 2′-deoxy-2′-fluoro (2′F) modified nucleotides.

In other non-limiting examples in which the internucleosidic linkage is 2′ to 5′ (rather than the standard 3′ to 5′), similar modifications to the sugar moiety as described herein can be made at the 3′ carbon in the sugar moiety. In a particular embodiment described herein, the sugar moiety of the described dsRNAs are modified to have H (instead of OH) at the 3′ position such that the 2′ position participates in the backbone formation.

### Nucleotide Modifications: Nitrogenous Base

In particular embodiments, the described double stranded nucleic acid can include a nucleotide comprising a modified base. Modified bases include but are not limited to synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

In other particular embodiments, the nucleotide is modified in that it is “abasic” and does not comprise a nitrogenous base at a particular position.

### Nucleotide Modifications: Internucleoside Linkage

A modified nucleotide of the described double stranded nucleic acids can also include modifications from the standard phosphodiester bond between two adjacent nucleotides (such modifications are also referred to as “internucleotide linkage” modifications or “backbone linkage” modifications). According to some embodiments, a nucleic acid molecule that comprises a modified internucleotide linkage refers to a molecule that includes at least one of: an internucleotide linkage other than a phosphodiester bond, an internucleotide linkage between positions other than 3′ and 5′ within the sugar moiety of neighboring nucleotides (such as an internucleotide linkage between position 2′ in a nucleotide's sugar moiety and position 5′ in the next nucleotide's sugar moiety), or a combination thereof.

In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (-0-Si(H)2-0-); sulfide, sulfoxide and sulfone linkages; formacetyl and thioformacetyl linkages; alkene containing backbones; sulfamate backbones; methylenemethylimino (—CH2-N(CH 3)-0-CH 2-) and methylenehydrazino linkages; sulfonate and sulfonamide linkages; amide linkages; and others having mixed N, O, S and CH 2 component parts.

According to another non-limiting example, the internucleotide linkage between two nucleotides in a nucleic acid may be substituted from a 3′-5′ linkage to a 2′-5′ bridged backbone linkage in which the 2′ position of the sugar moiety in the modified nucleotide is linked via a phosphodiester bond to the 5′ position of the sugar moiety of the next nucleotide (in this context, and as previously noted, “next” relates to the 5′>3′ direction, as commonly referred in the art), wherein such a modification may be referred to as a 5′-2′ bridge, a 2′-5′ bridge or a nucleotide-2′-phosphate (or a ribo-nucleotide-2′-phosphate in case of a modified ribonucleotide). In a particular embodiment, at least one internucleotide linkage between positions 6 and 7 or 7 and 8 from the 5′ terminus of the antisense strand of the described molecules is a 2′-5′ bridge. In a further particular embodiment, the internucleotide linkage between positions 7 and 8 is such a 2′-5′ bridge.

In particular embodiments, the described nucleic acids include an inverted RNA nucleotide, such as but not limited to at one or several of the strand ends. In particular examples the nucleic acid comprises at least an inverted nucleotide at the 3′ end of the first and/or the second strand and/or at the 5′ end of the second strand. An inverted nucleotide is a nucleotide that is linked to the 3′ end of a nucleic acid through its 3′ carbon, rather than its 5′ carbon as would normally be the case or is linked to the 5′ end of a nucleic acid through its 5′ carbon, rather than its 3′ carbon as would normally be the case.

In other embodiments, suitable RNA mimetics are contemplated for use in RNAi agents, in which both the sugar and the internucleoside linkage, i.e., the backbone, of the nucleotide units are replaced with alternate groups. The nucleobase units are maintained for hybridization with an appropriate nucleic acid target compound. One such oligomeric compound, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

### End Modifications

The double stranded nucleic acid compounds described herein can be a dsRNA compound that is blunt-ended on both sides of one or both strands, can have nucleotide or non-nucleotide overhangs on one or both termini of the sense strand (e.g., a 5′ cap and/or a 3′ overhang), can have nucleotide or non-nucleotide overhangs at the 3′ terminus of the antisense strand, or can have a combination of blunt and overhang termini within the requirements of the art. 5′ and 3′ modifications can be added to the described nucleic acids for a number of reasons, including to modulate activity, increase metabolic stability, or provide other functionality such as cellular targeting or labeling. Such modifications can include modification or replacement of an entire terminal phosphate or of one or more of the atoms of the phosphate group.

Terminal modifications useful for modulating activity and increasing stability include modification of the 5′ end with phosphate or phosphate analogues. The described dsRNAs can be 5′ phosphorylated or include a phosphoryl analogue at the 5′ prime terminus. 5′-phosphate modifications include 5′-monophosphate ((HO)2(O)P—O-5′); 5′-diphosphate ((HO)2(O)P—O—P(HO)(O)-0-5′); 5′-triphosphate ((HO)2(O)P—O—(HO)(O)P—O—P(HO)(O)-0-5′); 5′-guanosine cap (7-methylated or non-methylated) (7m-G-O-5′-(HO)(O)P—O—(HO)(O)P—Ov P(HO)(O)—0-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)2(O)P—NH-5′, (HO)(NH2)(O)P—O-5′), 5-alkylphosphonates (alkyl=methyl, ethyl, isopropyl, propyl, etc., e.g. RP(OH)(O)-0-5′- (wherein R is an alkyl), (OH)2(O)P-5′-CH2-), 5′ vinylphosphonate, 5′-alkyletherphosphonates (alkylether=methoxymethyl (MeOCH2-), ethoxymethyl, etc., e.g. RP(OH)(O)—O-5′- (wherein R is an alkylether)). Additional 5′ modifications at the antisense strand include 5′-POR and derivates thereof, described by Carrigan-Broda et al., 2023 RNA Therapeutics Conference, UMass. Other 5′ phosphate modifications within the scope of the present disclosure are described in US 2023/0346819, the contents of which are incorporated by reference herein.

In particular embodiments, the described dsRNAs include an antisense strand which has a terminal 5′ (E)-vinylphosphonate sugar modification at its 5′ end. In particular embodiments, the terminal 5′ (E)-vinylphosphonate nucleotide is linked to the second nucleotide in the sense strand by a 3′-5′ phosphodiester linkage. In still more particular embodiments, the terminal 5′ (E)-vinylphosphonate nucleotide is a uridine or adenine with a 2′ OMe or 2′F ribose modification.

In particular embodiments, the terminal 5′ (E)-vinylphosphonate nucleotide is linked to the second nucleotide in the sense strand by a 2′-5′ phosphodiester linkage. In still more particular embodiments, the terminal 5′ (E)-vinylphosphonate nucleotide is a uridine or adenine with a 3′ OMe or 3′F or 3′H ribose modification.

In particular embodiments, the 5′ and or 3′ end of the described dsRNA can include a a non-nucleotide moiety, such as a C3 alkyl or —C3-OH moiety. In particular embodiments, such covalently linked moieties can be a combination of one or more of the moieties described herein.

The capping and/or terminal moiety or moieties can include saturated aliphatic groups, including linear-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a linear chain or branched chain alkyl has 10 or fewer carbon atoms in its backbone (e.g., C1-C10 for linear chain, C3-C10 for branched chain), such as 6 or fewer carbon atoms. Likewise, particular cycloalkyls can have from 3-8 carbon atoms in their ring structure, such as 5 or 6 carbons in the ring structure. The term C1-C10 includes alkyl groups containing 1 to 10 carbon atoms.

In particular embodiments, the dsRNAs described herein include a C3 moiety at the 5′ end of the sense strand. “C3” and “C3 alkyl moiety” are used herein interchangeably and refer to an alkyl moiety, particularly a propanol or C3 alkyl moiety, wherein the C3 alkyl moiety can be covalently linked to a terminus of a dsRNA strand through a phosphate group (Pi) covalently linked to the 5′ position of the sugar moiety of the 5′ terminal nucleotide.

In a particular non-limiting embodiment, the C3 moiety is attached to the 5′ end of the sense strand as shown below, with the C3 moiety liked to the terminal phosphate circled:

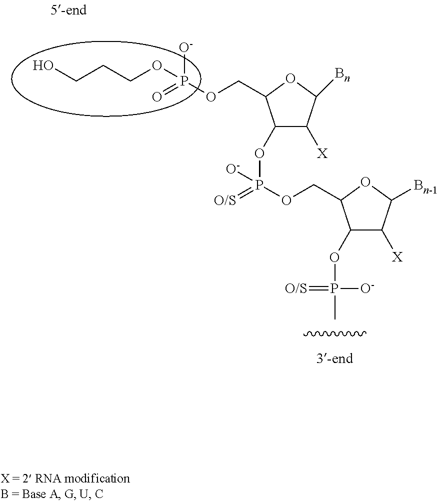

In particular embodiments, the 3′ terminus of a sense strand and/or an antisense strand include a C3-Pi or C4-Pi moiety covalently attached through a phosphate or a phosphorothioate group at the 3′ end. Such moieties can in certain embodiments be used to mimic nucleotide overhangs of 1, 2, 3 or more nucleotides. According to some embodiments, the 3′ terminus of the sense strand of the described dsRNA molecule, such as an siRNA compound, is covalently linked to a C3-Pi or C4-Pi moiety, wherein the C3-Pi or C4-Pi moiety is covalently linked to the 3′ terminus via a phosphodiester bond, and wherein the moiety comprises a free terminal phosphate group.

In other embodiments, the covalently-linked moiety includes two C3-Pi or C4-Pi moieties which are covalently linked via the phosphate group (Pi) of the first C3 (or C4) moiety, and in which there is a terminal phosphate group (the free phosphate of the second C3-Pi group). Such a C3/C4-Pi-C3/C4-Pi moiety is, in particular embodiments, covalently linked through a phosphate group to the 3′ terminus of the antisense and/or the sense strand of a dsRNA, or siRNA. In other particular embodiments, in non-nucleotide moieties comprising a C3 alkyl moiety, one or more of the Pi moieties may be replaced by a Ps moiety, which represents a phosphorothioate bond when covalently linking two moieties, or which represents a terminal phosphorothioate moiety when present in a 3′ terminus of sense and/or antisense strand in an siRNA.

### Targeting Ligands

In particular embodiments, the described double stranded nucleic acid molecules include at least one targeting ligand that is covalently attached to the double stranded nucleic acid. The targeting ligand can in certain embodiments be covalently attached to the 2′ position of a nucleotide sugar within the sense and/or antisense strands or loop forming nucleotides of the dsRNA or ssRNA siRNA molecule. In other embodiments, the targeting ligand is covalently attached to at least one of the 5′ end of the sense strand, the 3′ end of the sense strand, or the 3′ end of the antisense strand. In those embodiments wherein the targeting ligand is attached to only one of 3′ ends of the sense or antisense sequence, the 3′ end of the strand that does not comprise a targeting ligand is extended to have a 3′ overhang of 1, 2, 3, or more nucleotides, such as a 3′ di or trinucleotide overhang or a non-nucleotide overhang.

Particular non-limiting examples of targeting ligands that can be covalently attached to the described nucleic acid molecules include a vitamin or receptor binding small molecules of low molecular weight of any type, lipid or other lipophilic conjugate including but not limited to cholesterol, tocopherol or C16/C18 acyl fatty acid, or vitamins (see for illustrative examples, Do et al, ACS Omega 2017, 2, 6939-6957, the contents of which are incorporated by reference herein). Derivatives of the foregoing conjugates could also be used. In other embodiments, such conjugated moieties include specific cell receptor targeting moieties, e.g., peptide and polypeptide moieties including linear, cyclic, bicyclic or multicyclic peptides and polypeptides, antibodies of various types and forms including but not limited to complete antibodies, Fab's, minibodies, single-chain antibodies; targeting protein domains like e.g., centyrins, synthetic small molecules and aptamers. In a particular embodiment, antibodies of various types, peptides of various types, polypeptides or aptamers that bind to the TFR1 receptor can be covalently attached to the described molecules for delivery of the described nucleic acids to erythrocytes, muscle and CNS (via a blood brain barrier).

Other targeting ligands that can be covalently attached to the described molecules can be carbohydrate (i.e., glycan)-based ligands including but not limited to N-acetylgalactosamine (GalNAc), mannose, fucose, galactose, N-Acetylneuraminic acid (Neu5Ac), N-acetylglucosamine (GlcNAc), folic acid and their derivatives (see for illustrative examples, Uehara et al., Nucl. Acid Res. 2022 May 20; 50(9): 4840-4859, the contents of which are incorporated by reference herein). Other particular examples of glycan ligands are those that bind to the mannose receptor on a variety of cell types (Cummings, Curr. Opin. Struct. Biol. 75:102394, 2022, the contents of which are incorporated by reference herein).

The targeting ligands described herein can be incorporated with the described dsRNAs by any method known to the art, such as during automatic nucleic acid synthesis as an amidite or linked to the column material or in other embodiments can be added using post-synthesis conjugation using methods known in the art.

In particular embodiments, the targeting ligand comprises at least one N-acetylgalactosamine (GalNAc) moiety, and more particularly two, three or four (see International Patent Publication No. WO 2012/177921 for non-limiting examples of GalNAc targeting ligands).

In particular embodiments of the described molecules, the targeting ligand is covalently attached to the described nucleic acid at the desired location via a phosphodiester or a phosphorothioate bond during an automatic oligonucleotide synthesis being a part of a phosphoroamidite. Such phosphoroamidites can be either directly attached to the nucleotide entity, or can be separated from the nucleotide by a linker from the 5′ end, and/or the 3′ end, and/or being covalently attached to a 2′ position of the ribose sugar (e.g., in those embodiments in which the ligand is a part of a nucleoside-containing phosphoroamidite). Linkers for use with the described molecules include but are not limited to a chemically modified nucleotide linker (1, 2, 3, or more nucleotides), or a chemical linker, such as e.g., an alkyl linker. The linkers connecting the targeting moiety to the described nucleic acids can also vary as well as the core structures of phosphoroamidites. Particular non-limiting examples of chemical linkers through which a targeting moiety can be attached to a phosphoroamidite include but are not limited to polyethylenglycol (PEG), triethyleneglycol (TEG), C4 urea, C6 carbamate and the like (for non-limiting examples, see International Patent Publication Nos. WO 2018/013999 and WO 2012/177921, the contents of which are incorporated by reference herein with respect to targeting ligands and linkers).

In a particular embodiment, the targeting ligand is linked to the described nucleic acids via a TEG linker that is 2-(6-(bis(4-methoxyphenyl)(phenyl)methoxy)-5-(hydroxymethyl)hexyl)isoindoline-1,3-dione linker available for example from Hongene Biotech (Cat. No. ON-153).

In another embodiment, the double stranded nucleic acid has three or more GalNAc moieties covalently bound to the 3′ end of the sense strand. GalNAc moieties in the described dsRNA molecules can in particular embodiments be added sequentially as monomers during oligonucleotide synthesis being introduced in the form of phosphoroamidites or immobilized on glass or polystyrene columns to allow for oligonucleotide initiation. In such embodiments, the individual GalNAc moieties are connected between them and/or to the oligonucleotide terminus via phosphodiester bonds that can be also modified to have e.g., phosphorothioate or other types of backbone modifications as described herein. In a particular embodiment, the double stranded nucleic acid has three or more GalNAc moieties individually connected to three or four core moieties by way of a triethylene-glycol linkers in which the first GalNAc structure is immobilized, the next two or three are sequentially added and then the nucleotides are added during the remainder of oligonucleotide synthesis. Other methodologies for adding such ligands to the described nucleic acids are also encompassed by the current disclosure.

In a particular embodiment, the ligand is a series of three GalNAc monomers with a triethyleneglycol (TEG) linker, and is connected to the 3′ end of the sense strand. An illustration of one such ligand is shown below:

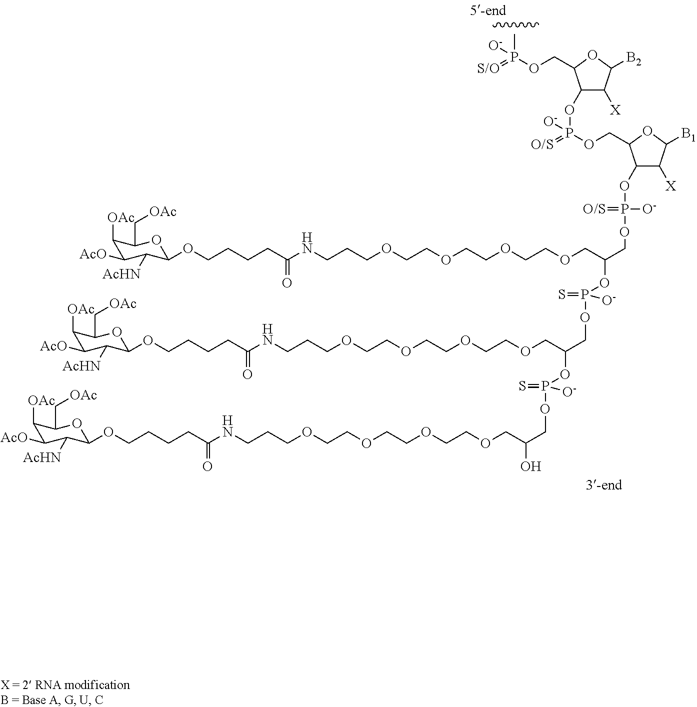

### Nucleic Acid Compositions

The synthetic double stranded nucleic acids described herein can be synthesized by any standard method known in the art for synthesis of ribonucleic (or deoxyribonucleic) oligonucleotides. These procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired (see for example, Hughes, Cold Spring Harbor Perspect. Biol. 9, 2017; Sandahl et al., Nature Comm. 12, 2021; and Brazier, Methods Mol. Bio. Vol. 2633, 2023). The nucleic acid molecules disclosed herein can be synthesized separately and as relevant hybridized following synthesis and/or deprotection.

The double stranded nucleic acids (e.g., dsRNA and more particularly siRNA) and pharmaceutically acceptable salts thereof described herein can, in particular embodiments, be delivered to the target tissue, such as liver tissue, by direct administration of the “naked” molecules. The terms “naked nucleic acid” or “naked dsRNA” or “naked siRNA” refers to nucleic acid molecules that are free from any delivery vehicle that acts to assist, promote, or facilitate entry into the cell such as e.g., lipid or polymer nanoparticle formulations. For example, dsRNA (with or without covalently attached targeting ligand) in PBS is “naked dsRNA”.

In other embodiments, the described dsRNA compounds, pharmaceutically acceptable salts thereof, and pharmaceutical compositions thereof, can include a pharmaceutically-acceptable delivery vehicle, including one or more excipients, carriers, buffers, or diluents. The pharmaceutical compositions described herein can additionally or alternatively be formulated as a lipid or polymer nanoparticle formulation and the like for administration to a subject, and to facilitate entry of the dsRNA into the target organ, tissue, and cell.

The pharmaceutically acceptable carriers, buffers, solvents, diluents, excipients, adjuvants and vehicles generally refer to inert, non-toxic solid or liquid fillers, diluents or encapsulating material not reacting with the active ingredients disclosed herein. The pharmaceutically acceptable carriers and pharmaceutically acceptable salts useful in this disclosure are conventional. Remington (The Science and Practice of Pharmacology, 23rd Edition (2020)), describes compositions and formulations suitable for pharmaceutical delivery of the compounds herein disclosed. In general, the nature of the carrier and/or salt will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle.

Additional illustrative examples of pharmaceutical nucleic acid formulations and methods of their delivery can be found in Roberts et al., Nature Rev. Drug Disc. 19:673-694, 2020; and Hammond et al., EMBO Mol. Med., 13(4), 2021, the contents of both of which are incorporated herein by reference.

In addition, as described below, double stranded nucleic acids described and claimed herein are demonstrated to possess a superior ability to knock down TFR2 mRNA in formulations that do not include a transfection mediating agent, such as a lipid-based transfection agent.

Treatment of Hepcidin-Related Diseases with Nucleic Acids that Inhibit TFR2 Expression

The described nucleic acid compositions can be used in methods for treatment of diseases associated with increased circulating levels of hepcidin (a protein product of the HAMP gene, the expression of which is regulated by TFR2 expression), e.g., due to increased expression of the HAMP gene in hepatocytes and/or reduced glomerular filtration of hepcidin as occurring during kidney malfunction. The methods involve administration to a subject in need thereof an effective amount of a TFR2 mRNA-targeting double stranded nucleic acid described herein or pharmaceutical compositions thereof, including pharmaceutical compositions that do not include a transfection mediating agent, such as a lipid-based transfection agent, thereby knocking down TFR2 expression in hepatocytes and treating the condition associated with increased circulating levels of hepcidin. Such compositions can be considered “therapeutically relevant” in their ability reduce hepcidin levels as a consequence of knocking down TFR2 mRNA using a physiologically compatible composition (including transfection-agent free compositions), thereby treating a hepcidin-related disorder, including HAMP-related anemia. In particular embodiments the TFR2-targeting double stranded nucleic acid is administered subcutaneously.

TFR2 expression in erythrocytes is also involved in control of erythropoiesis, with inhibition of TFR2 expression acting to increase expression of erythropoietin receptors on the cell surface, and thereby stimulate erythropoiesis (Richard et al., Int. J. Mol. Sci. 21:9173, 2020). Therefore, in particular embodiments, in which the described nucleic acids are targeted to erythrocytes by way of a TFR1-targeting ligand such as but not limited to a TFR1-binding antibody, the described nucleic acids can be used in methods to stimulate erythropoiesis and thereby treat anemia.

As described, aberrant overexpression of HAMP is associated, inter alia, with iron-deficiency related anemias, which are described collectively herein as “HAMP-related anemias” or alternatively “HAMP-associated anemias”. In particular embodiments, the subject to be treated has an iron-deficient anemia, including but not limited to iron-refractory anemia (both hereditary and acquired) or anemia of a chronic disease and/or inflammation. Particular non-limiting examples of such anemias include anemias arising from cancer, including solid tumors, and cancers at an advanced stage. Particular embodiments include hematology-oncology related indications, including anemia associated with myelofibrosis (transfusion dependent and transfusion independent), multiple myeloma, Castelman disease, lymphoma (Hodgkin lymphoma and non-Hodgkin lymphoma), leukemias such as but not limited to chronic lymphocytic leukemia, myelodysplastic syndrome (MDS), myeloproliferative neoplasms (MPN), and Waldenstrom macroglobulinemia.

As noted, in particular embodiments, anemias for treatment with the compositions described herein include anemias occurring in patients with cancer (such as but not limited to myeloproliferative neoplasms such as myelofibrosis) or arising from the cancer itself. In other embodiments, the described compositions can be used for treatment of non-cancer related anemias related to inflammation, such as but not limited to anemia in patients with rheumatoid arthritis, inflammatory bowel disease, autoimmune conditions including but not limited to systemic lupus erythematosus (SLE), Scleroderma Psoriatic arthritis, Vasculitides, autoimmune thyroiditis, Anti-Neutrophil Cytoplasmic Antibody (ANCA) associated vasculitis, kryoglobulinemia, chronic kidney disease, chronic inflammation, such as in HIV infected patients, obesity, metabolic syndrome, chronic infections (such as but not limited to tuberculosis and osteomyelitis), chronic obstructive pulmonary disease, and chronic graft versus host disease (GVHD; post-hematopoietic stem cell transplant).

In particular embodiments, the anemia arises from a hereditary disorder, and is a hereditary iron deficiency anemia resulting in high hepcidin. Particular non-limiting examples include an Iron-refractory iron deficiency anemia (IRIDA), or an anemia arising from mutations in the TMPRSS6 gene.

In other embodiments, the subject has a condition that results in overproduction or reduced elimination of hepcidin, such as chronic kidney disease and Hepcidin producing hepatocellular adenoma.

In particular embodiments, the described compositions can be used for treatment of a subject that is receiving treatment for an anemia, such as with an Erythropoietin-Stimulating Agent (ESA), yet is non- or insufficiently responsive to the treatment. In such embodiments the described compositions can be administered sequentially (either before or after) or concurrently with the anemia treatment, or following a determination of treatment non- or poor response.

In still other embodiments, the compositions described herein can be used to treat an subject with a condition who develops an anemia that is multifactorial, and can be one of several physiological pathologies associated with the condition, in which oral iron absorption is limited but intravenously administered iron has been shown to impact the treatment outcome. Such conditions include congestive Heart Failure, COVID (acute and long-COVID), pregnancy, anemic conditions arising from surgery, advanced age, and conditions arising from prolonged hospitalization following surgery and/or trauma and intensive care, such as sepsis and multi-organ failure.

In other embodiments, the subject in need of the treatments described herein has iron-restricted erythropoiesis or another condition related to reduced iron levels. In still other embodiments, the subject has a condition for which an increase in iron levels (resultant from TFR2 knockdown and associated reduction of hepcidin levels) would provide a therapeutic benefit.

Pharmaceutical compositions of the described dsRNAs are detailed in the preceding section and can be used alone or in combination with one or more additional therapeutic agents. A combination therapy may include at least one of the described double stranded nucleic acids combined with at least one other therapeutic agent that can be used to treat a disease or condition related to iron deficiency with high hepcidin levels such as an anemia. Such agents include but are not limited to therapeutically active small molecules or polypeptides, a single chain antibody, a classical antibody or fragment thereof, or an additional synthetic nucleic acid molecule which targets the expression of an alternative TFR2 sequence or one or more additional genes in the same or relevant associated pathways controlling iron homeostasis. In particular embodiments, such additional synthetic nucleic acids can be covalently joined with TFR2 siRNA to form a bi- or multivalent specific molecule. In other embodiments, such synthetic nucleic acids can be administered independently or in a form of defined combination formulations. In still other embodiments, each strand of the described double stranded nucleic acids can be provided in separate pharmaceutical compositions to the subject.

It will be understood that targeting and inhibiting TFR2 expression in the described methods does not require that 100% of the TFR2 mRNA be knocked down in the targeted organ or tissue, such as the liver (more specifically, hepatocytes). Rather, the described methods require at least partial inhibition of TFR2 expression, such that the expression of TFR2, and by extension HAMP, is from 0% to less than 100% of the expression that would be present in the absence of the described dsRNA compound. In particular embodiments, less than 100%, less than 90%, less than 80%, less than 70%, less than 60%, less than 50%, less than 40%, less than 30%, less than 20%, or even less than 10% of TFR2 expression is necessary to achieve the desired therapeutic effect.

The compositions and pharmaceutical compositions described herein can be administered to a subject in need of the described treatments by any route known to one of skill in the art for providing nucleic acids or nucleic acid-associated materials to a subject, including by way of a delivery device employed for or adapted for delivery of nucleic acids to a subject. Administration can be local or systemic. Local administration of the described compositions includes direct administration to a target organ or tissue. Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system. In particular embodiments, the described compositions are administered by subcutaneous injection.

The dsRNAs for use in the described methods of treatment are provided in an effective amount which can also be understood to be a “therapeutically effective amount.” Such amounts include a quantity of the described compound sufficient to achieve a desired therapeutic effect in a subject being treated. For example, an amount of TFR2 targeting dsRNA necessary to decrease HAMP expression to sufficiently increase iron availability in the subject for hematopoiesis. The effective amount of the compound will be dependent on the compound applied, the subject being treated, and particularly factors such as subject weight and metabolism, the severity and type of the affliction.

The methods of treatment described herein are designed to treat or even prevent pathological conditions such as iron deficiency related anemias in a subject. Preventing a disease can be complete or even partial prevention of the disease, such as the anemia. Partial prevention of a disease is when a given treatment leads to its development in a milder form and/or development of the disease or condition only after a longer period of time. Treatment refers to a therapeutic intervention that ameliorates or reduces a sign or symptom of a disease or pathological condition after it has begun to develop. As used herein, the term “subject” includes human and non-human mammals.

The following examples are provided to illustrate certain particular features and/or embodiments. These examples should not be construed to limit the disclosure to the particular features or embodiments described. Nor should these examples be construed to limit the disclosure to particular materials and methods for use in synthesis of the nucleic acids described herein.

### EXAMPLES

The following examples describe the discovery of siRNAs for knocking down the expression of human TFR2 mRNA with overall superior properties of robust on-target knockdown activity in vitro and in vivo, and with minimal to no seed-mediated off-target activity in an in vitro model. Examples 1-6 present a screen for TFR2 mRNA-targeting siRNAs and demonstrate that siRNAs having sense strands with a 5′ nucleotide corresponding to position 2657 of the TFR2 mRNA, in which the 5′ nucleotide of the sense strand sequence is at position 2657 according to NCBI accession number NM_001206855.3 (SEQ ID NO:305) (TFR2-2657 siRNAs), are among those with overall superior properties.

### Example 1: Screening of the Initial Set of TFR2 siRNAs in HepG2 Cells

To identify potent siRNAs for inhibiting TFR2 mRNA expression, a series of fully chemically modified siRNAs (Table 2) was designed and synthesized on an automatic oligonucleotide synthesizer as follows and as further detailed in Example 3. Single strands (i.e., sense and antisense strands) were synthesized according to conventional solid-phase oligonucleotide synthesis methodology using standard phosphoramidite-based oligomerization chemistry whereby the oligonucleotides were assembled on solid support, employing a Mermade 96E synthesizer (LGC Bioautomation) in a 96 well format controlled by the Poseidon software package.

For the screening purposes, all siRNAs have been synthesized with the same pattern of chemical modifications. The siRNA positions provided in Table 2 (“Position” column) correspond to the position of the 5′ nucleotide of the sense strands according to the human TFR2 mRNA (cDNA) sequence available at NCBI accession number NM_001206855.3 (SEQ ID NO:305).

As noted above in Table 1, the described sequences include the following modifications: Nf: 2′-Fluoro (2′F) RNA nucleotides; n: 2′-O-Methyl (2′OMe) RNA nucleotides; (C3): propanol or C3 alkyl moiety connected to the 5′ end of the strand via a phosphodiester linkage; s: Phosphorothioate (Ps) backbone modification.

TABLE 2
TFR2-targeting siRNAs for initial activity screen

| SEQ | Starting | | | |
|---|---|---|---|---|
| ID NOs | nt | siRNA ID | sense strand | antisense strand |
| 1, 2 | 39 | XD-52020 | (C3)csasAfgcaUfGfGfagCfggcuUfua | usAfsaagccgcuccaUfgcuugsusg |
| 3, 4 | 65 | XD-52021 | (C3)ususCfcagAfGfAfgcGfcaacAfaa | usUfsuguugcgcucuCfuggaasusa |
| 5, 6 | 66 | XD-52022 | (C3)uscsCfagaGfAfGfcgCfaacaAfca | usGfsuuguugcgcucUfcuggasasu |
| 7, 8 | 67 | XD-52023 | (C3)cscsAfgagAfGfCfgcAfacaaCfua | usAfsguuguugcgcuCfucuggsasa |
| 9, 10 | 68 | XD-52024 | (C3)csasGfagaGfCfGfcaAfcaacUfga | usCfsaguuguugcgcUfcucugsgsa |
| 11, 12 | 69 | XD-52025 | (C3)asgsAfgagCfGfCfaaCfaacuGfua | usAfscaguuguugcgCfucucusgsg |
| 13, 14 | 88 | XD-52026 | (C3)cscsCfaagAfUfCfcuCfucagAfca | usGfsucugagaggauCfuugggsgsa |
| 15, 16 | 92 | XD-52027 | (C3)asgsAfuccUfCfUfcaGfaccgUfca | usGfsacggucugagaGfgaucususg |
| 17, 18 | 97 | XD-52030 | (C3)csusCfucaGfAfCfcgUfcuacCfaa | usUfsgguagacggucUfgagagsgsa |
| 19, 20 | 98 | XD-52031 | (C3)uscsUfcagAfCfCfguCfuaccAfga | usCfsugguagacgguCfugagasgsg |
| 21, 22 | 99 | XD-52032 | (C3)csusCfagaCfCfGfucUfaccaGfca | usGfscugguagacggUfcugagsasg |
| 23, 24 | 241 | XD-52035 | (C3)csasGfgcaGfCfCfaaAfccucAfua | usAfsugagguuuggcUfgccugsgsg |
| 25, 26 | 387 | XD-52041 | (C3)usgsUfuggUfGfGfucAfgugaGfga | usCfscucacugaccaCfcaacascsa |
| 27, 28 | 406 | XD-52042 | (C3)usgsUfcaaCfUfAfugAfgccuGfaa | usUfscaggcucauagUfugacasusc |
| 29, 30 | 409 | XD-52043 | (C3)csasAfcuaUfGfAfgcCfugacCfua | usAfsggucaggcucaUfaguugsasc |
| 31, 32 | 412 | XD-52044 | (C3)csusAfugaGfCfCfugAfccugGfaa | usUfsccaggucaggcUfcauagsusu |
| 33, 34 | 721 | XD-52051 | (C3)gsgsAfggaCfCfCfugAfcgucUfaa | usUfsagacgucagggUfccuccsasg |
| 35, 36 | 871 | XD-52054 | (C3)gsgsUfgauCfAfGfcuUfcgccCfaa | usUfsgggcgaagcugAfucaccscsc |
| 37, 38 | 885 | XD-52057 | (C3)cscsCfagaAfGfGfugAfccaaUfga | usCfsauuggucaccuUfcugggscsg |
| 39, 40 | 888 | XD-52058 | (C3)asgsAfaggUfGfAfccAfaugcUfca | usGfsagcauuggucaCfcuucusgsg |
| 41, 42 | 892 | XD-52059 | (C3)gsgsUfgacCfAfAfugCfucagGfaa | usUfsccugagcauugGfucaccsusu |
| 43, 44 | 895 | XD-52060 | (C3)gsasCfcaaUfGfCfucAfggacUfua | usAfsaguccugagcaUfuggucsasc |
| 45, 46 | 997 | XD-52063 | (C3)asgsUfguaUfGfGfacAfugugCfaa | usUfsgcacauguccaUfacacusgsc |
| 47, 48 | 1051 | XD-52065 | (C3)ususCfcuuCfAfAfucAfaaccCfaa | usUfsggguuugauugAfaggaasgsg |
| 49, 50 | 1051 | XD-52066 | (C3)gsusCfcuuCfAfAfucAfaaccCfaa | usUfsggguuugauugAfaggacsgsg |
| 51, 52 | 1053 | XD-52067 | (C3)cscsUfucaAfUfCfaaAfcccaGfua | usAfscuggguuugauUfgaaggsasa |
| 53, 54 | 1054 | XD-52068 | (C3)csusUfcaaUfCfAfaaCfccagUfua | usAfsacuggguuugaUfugaagsgsa |
| 55, 56 | 1055 | XD-52070 | (C3)ususCfaauCfAfAfacCfcaguUfca | usGfsaacuggguuugAfuugaasgsg |
| 57, 58 | 1303 | XD-52079 | (C3)csusCfagaGfCfCfagAfucacUfaa | usUfsagugaucuggcUfcugagscsg |
| 59, 60 | 1445 | XD-52084 | (C3)csgsCfagaAfGfUfcuCfcucuUfca | usGfsaagaggagacuUfcugcgsgsg |
| 61, 62 | 1448 | XD-52085 | (C3)asgsAfaguCfUfCfcuCfuucaUfca | usGfsaugaagaggagAfcuucusgsc |
| 63, 64 | 1531 | XD-52089 | (C3)gscsUfgcaCfCfUfcaAfagccGfua | usAfscggcuuugaggUfgcagcsasc |
| 65, 66 | 1534 | XD-52090 | (C3)gscsAfccuCfAfAfagCfcguaGfua | usAfscuacggcuuugAfggugcsasg |
| 67, 68 | 1536 | XD-52091 | (C3)ascsCfucaAfAfGfccGfuaguGfua | usAfscacuacggcuuUfgaggusgsc |
| 69, 70 | 1537 | XD-52092 | (C3)cscsUfcaaAfGfCfcgUfagugUfaa | usUfsacacuacggcuUfugaggsusg |
| 71, 72 | 1539 | XD-52093 | (C3)uscsAfaagCfCfGfuaGfuguaCfga | usCfsguacacuacggCfuuugasgsg |
| 73, 74 | 1542 | XD-52094 | (C3)asasGfccgUfAfGfugUfacguGfaa | usUfscacguacacuaCfggcuususg |
| 75, 76 | 1546 | XD-52095 | (C3)csgsUfaguGfUfAfcgUfgagcCfua | usAfsggcucacguacAfcuacgsgsc |
| 77, 78 | 1549 | XD-52096 | (C3)asgsUfguaCfGfUfgaGfccugGfaa | usUfsccaggcucacgUfacacusasc |
| 79, 80 | 1581 | XD-52099 | (C3)gsgsGfaugAfCfAfagUfuucaUfga | usCfsaugaaacuuguCfaucccscsc |
| 81, 82 | 1588 | XD-52100 | (C3)csasAfguuUfCfAfugCfcaagAfca | usGfsucuuggcaugaAfacuugsusc |
| 83, 84 | 1616 | XD-52105 | (C3)csusGfacaAfGfUfcuCfauugAfga | usCfsucaaugagacuUfgucagsasa |
| 85, 86 | 1617 | XD-52106 | (C3)usgsAfcaaGfUfCfucAfuugaGfaa | usUfscucaaugagacUfugucasgsa |
| 87, 88 | 1619 | XD-52107 | (C3)ascsAfaguCfUfCfauUfgagaGfua | usAfscucucaaugagAfcuuguscsa |
| 89, 90 | 1626 | XD-52108 | (C3)uscsAfuugAfGfAfguGfuccuGfaa | usUfscaggacacucuCfaaugasgsa |
| 91, 92 | 1627 | XD-52109 | (C3)csasUfugaGfAfGfugUfccugAfaa | usUfsucaggacacucUfcaaugsasg |
| 93, 94 | 1670 | XD-52115 | (C3)gsgsGfcagAfCfUfcuCfuaugAfaa | usUfsucauagagaguCfugcccsasc |
| 95, 96 | 1671 | XD-52116 | (C3)gsgsCfagaCfUfCfucUfaugaAfca | usGfsuucauagagagUfcugccscsa |
| 97, 98 | 1675 | XD-52120 | (C3)gsasCfucuCfUfAfugAfacagGfua | usAfsccuguucauagAfgagucsusg |
| 99, 100 | 1686 | XD-52125 | (C3)asasCfaggUfGfGfugUfucacCfaa | usUfsggugaacaccaCfcuguuscsa |
| 101, 102 | 1836 | XD-52130 | (C3)ascsAfcaaAfGfGfagGfacacUfua | usAfsaguguccuccuUfugugusgsc |
| 103, 104 | 1837 | XD-52131 | (C3)csasCfaaaGfGfAfggAfcacuUfaa | usUfsaaguguccuccUfuugugsusg |
| 105, 106 | 1840 | XD-52134 | (C3)asasAfggaGfGfAfcaCfuuauGfaa | usUfscauaaguguccUfccuuusgsu |
| 107, 108 | 1840 | XD-52135 | (C3)gsasAfggaGfGfAfcaCfuuauGfaa | usUfscauaaguguccUfccuucsgsu |
| 109, 110 | 1999 | XD-52137 | (C3)gscsAfcauCfGfGfgaAfccucAfaa | usUfsugagguucccgAfugugcscsu |
| 111, 112 | 2004 | XD-52139 | (C3)uscsGfggaAfCfCfucAfacgaGfua | usAfscucguugagguUfcccgasusg |
| 113, 114 | 2110 | XD-52145 | (C3)gscsAfggaGfAfUfcuAfcagcUfca | usGfsagcuguagaucUfccugcscsg |
| 115, 116 | 2134 | XD-52155 | (C3)gsasGfagaCfGfAfgcGfacugAfca | usGfsucagucgcucgUfcucucsusc |
| 117, 118 | 2135 | XD-52156 | (C3)asgsAfgacGfAfGfcgAfcugaCfaa | usUfsgucagucgcucGfucucuscsu |
| 119, 120 | 2136 | XD-52157 | (C3)gsasGfacgAfGfCfgaCfugacAfca | usGfsugucagucgcuCfgucucsusc |
| 121, 122 | 2148 | XD-52158 | (C3)usgsAfcacGfCfAfugUfacaaCfga | usCfsguuguacaugcGfugucasgsu |
| 123, 124 | 2154 | XD-52159 | (C3)gscsAfuguAfCfAfacGfugcgCfaa | usUfsgcgcacguuguAfcaugcsgsu |
| 125, 126 | 2157 | XD-52160 | (C3)usgsUfacaAfCfGfugCfgcauAfaa | usUfsuaugcgcacguUfguacasusg |
| 127, 128 | 2158 | XD-52161 | (C3)gsusAfcaaCfGfUfgcGfcauaAfua | usAfsuuaugcgcacgUfuguacsasu |
| 129, 130 | 2168 | XD-52162 | (C3)csgsCfauaAfUfGfcgGfguggAfga | usCfsuccacccgcauUfaugcgscsa |
| 131, 132 | 2169 | XD-52163 | (C3)gscsAfuaaUfGfCfggGfuggaGfua | usAfscuccacccgcaUfuaugcsgsc |
| 133, 134 | 2172 | XD-52164 | (C3)usasAfugcGfGfGfugGfaguuCfua | usAfsgaacuccacccGfcauuasusg |
| 135, 136 | 2175 | XD-52166 | (C3)usgsCfgggUfGfGfagUfucuaCfua | usAfsguagaacuccaCfccgcasusu |
| 137, 138 | 2176 | XD-52167 | (C3)gscsGfgguGfGfAfguUfcuacUfua | usAfsaguagaacuccAfcccgcsasu |
| 139, 140 | 2177 | XD-52168 | (C3)csgsGfgugGfAfGfuuCfuacuUfca | usGfsaaguagaacucCfacccgscsa |
| 141, 142 | 2181 | XD-52169 | (C3)usgsGfaguUfCfUfacUfuccuUfua | usAfsaaggaaguagaAfcuccascsc |
| 143, 144 | 2188 | XD-52170 | (C3)csusAfcuuCfCfUfuuCfccagUfaa | usUfsacugggaaaggAfaguagsasa |
| 145, 146 | 2192 | XD-52173 | (C3)ususCfcuuUfCfCfcaGfuacgUfga | usCfsacguacugggaAfaggaasgsu |
| 147, 148 | 2192 | XD-52174 | (C3)gsusCfcuuUfCfCfcaGfuacgUfga | usCfsacguacugggaAfaggacsgsu |
| 149, 150 | 2193 | XD-52175 | (C3)uscsCfuuuCfCfCfagUfacguGfua | usAfscacguacugggAfaaggasasg |
| 151, 152 | 2401 | XD-52179 | (C3)asgsCfcaaUfGfCfgcUfuagcGfga | usCfscgcuaagcgcaUfuggcusgsc |
| 153, 154 | 2424 | XD-52180 | (C3)uscsUfggaAfCfAfuuGfauaaCfaa | usUfsguuaucaauguUfccagascsa |
| 155, 156 | 2425 | XD-52181 | (C3)csusGfgaaCfAfUfugAfuaacAfaa | usUfsuguuaucaaugUfuccagsasc |
| 157, 158 | 2427 | XD-52184 | (C3)gsgsAfacaUfUfGfauAfacaaCfua | usAfsguuguuaucaaUfguuccsasg |
| 159, 160 | 2429 | XD-52185 | (C3)asasCfauuGfAfUfaaCfaacuUfca | usGfsaaguuguuaucAfauguuscsc |
| 161, 162 | 2429 | XD-52186 | (C3)gsasCfauuGfAfUfaaCfaacuUfca | usGfsaaguuguuaucAfaugucscsc |
| 163, 164 | 2430 | XD-52187 | (C3)ascsAfuugAfUfAfacAfacuuCfua | usAfsgaaguuguuauCfaaugususc |
| 165, 166 | 2432 | XD-52188 | (C3)asusUfgauAfAfCfaaCfuucuGfaa | usUfscagaaguuguuAfucaausgsu |
| 167, 168 | 2432 | XD-52189 | (C3)gsusUfgauAfAfCfaaCfuucuGfaa | usUfscagaaguuguuAfucaacsgsu |
| 169, 170 | 2433 | XD-52190 | (C3)ususGfauaAfCfAfacUfucugAfga | usCfsucagaaguuguUfaucaasusg |
| 171, 172 | 2433 | XD-52191 | (C3)gsusGfauaAfCfAfacUfucugAfga | usCfsucagaaguuguUfaucacsusg |
| 173, 174 | 2543 | XD-52194 | (C3)ascsCfucuCfAfUfugCfugauCfaa | usUfsgaucagcaaugAfgaggusgsg |
| 175, 176 | 2605 | XD-52195 | (C3)asgsCfacaGfAfUfauCfcacaCfaa | usUfsguguggauaucUfgugcusgsg |
| 177, 178 | 2657 | XD-52197 | (C3)csgsGfucaUfAfCfugUfcgguUfaa | usUfsaaccgacaguaUfgaccgsusc |
| 179, 180 | 2663 | XD-52198 | (C3)usasCfuguCfGfGfuuAfaucaGfaa | usUfscugauuaaccgAfcaguasusg |
| 181, 182 | 2663 | XD-52199 | (C3)gsasCfuguCfGfGfuuAfaucaGfaa | usUfscugauuaaccgAfcagucsusg |
| 183, 184 | 2720 | XD-52200 | (C3)gsgsGfuccUfCfCfauAfccuaGfaa | usUfscuagguauggaGfgacccscsa |
| 185, 186 | 2792 | XD-52202 | (C3)asgsAfgauCfGfCfugGfcaccAfua | usAfsuggugccagcgAfucucuscsc |
| 187, 188 | 2793 | XD-52203 | (C3)gsasGfaucGfCfUfggCfaccaUfaa | usUfsauggugccagcGfaucucsusc |
| 189, 190 | 2800 | XD-52204 | (C3)csusGfgcaCfCfAfuaGfccuuAfua | usAfsuaaggcuauggUfgccagscsg |
| 191, 192 | 2806 | XD-52205 | (C3)cscsAfuagCfCfUfuaUfggccAfaa | usUfsuggccauaaggCfuauggsusg |

The activity of the siRNAs listed in Table 2 was tested as follows. HepG2 cells were obtained from ATCC (ATCC in partnership with LGC Standards, Wesel, Germany, cat. #ATCC-HB-8065) and cultured in MEM (#21090-022, Thermo Invitrogen, Germany), supplemented to contain 10% fetal calf serum (1248D, Biochrom GmbH, Berlin, Germany), 2 mM L-Glu (K0283, Biochrom GmbH, Berlin, Germany), 1× non-essential amino acids (K0293, Biochrom GmbH, Berlin, Germany), 1 mM Na-Pyruvate and 100 U/ml Penicillin/100 μg/ml Streptomycin (A2213, Biochrom GmbH, Berlin, Germany) at 37° C. in an atmosphere with 5% CO2 in a humidified incubator.

For transfection of HepG2 cells with siRNAs, cells were seeded at a density of 20,000 cells/well into 96-well tissue culture plates (GBO, Germany). Transfection of siRNA was carried out with Lipofectamine RNAiMax (Invitrogen/Life Technologies) according to manufacturer's instructions for reverse transfection. The dual dose screen was performed with TFR2 siRNAs in quadruplicates at 10 nM and 0.5 nM concentrations. siRNAs targeting AHSA1, Firefly-Luciferase and Factor VII and a mock transfection served as unspecific controls. After 24 h of incubation with siRNAs, culture medium was removed and cells were lysed with the lysis buffer included in the MagnaPure LC RNA isolation system (Roche, Cat #05467535001). RNA extraction was performed on the MagnaPure 96 instrument, following the manufacturer's instructions. cDNA was generated using the high-capacity reverse transcription kit (ThermoFisher, Cat #4368813). cDNA corresponding to approximately 40 ng of RNA was used for the qPCR reaction. qPCR was performed using the TaqMan fast advanced MasterMix (ThermoFisher, Cat #4444557), together with primer pairs for TFR2 (labeled with FAM dye; ThermoFisher, #Hs00162690_m1-FAM) and GAPDH (labeled with VIC dye; ThermoFisher, #4326317) in one reaction. The AHSA1-siRNA served at the same time as an unspecific control for TFR2 mRNA expression and as a positive control to analyze transfection efficiency with regards to AHSA1 mRNA level by qPCR analysis with an AHSA1 primer set for each transfection plate (ThermoFisher, #Hs00201602_m1-FAM). Transfection efficiency for each 96-well plate was calculated by analysis of AHSA1-knock-down with AHSA1-siRNA (normalized to GapDH) compared to an unspecific control.

For data evaluation, in each well of the experiments, TFR2 mRNA levels were normalized to the respective GAPDH mRNA level (calculating ACT) and then the activity of a given TFR2 siRNA was expressed as percent TFR2 mRNA concentration (normalized to GAPDH mRNA) in treated cells, relative to the TFR2 mRNA concentration (normalized to GAPDH mRNA) averaged across control wells, using the formula 2 exp(−ΔΔCₜ).

Table 3 shows the results of this “dual-dose” experiment as % residual TFR2 mRNA expression in siRNA-treated cells compared to mock-transfected cells (100%). Mean values (%) and standard deviation (SD) per each quadruplicate are shown.

TABLE 3
Results of the dual dose screening of TFR2-targeting
siRNAs in HepG2 cells

| | | TFR2 mRNA KD (% residual) | | | |
|---|---|---|---|---|---|
| | | 10 nM | | 0.5 nM | |
| | Starting | Mean | SD | Mean | SD |
| siRNA ID | nt | (%) | (%) | (%) | (%) |
| XD-52020 | 39 | 24.17 | 2.05 | 43.02 | 6.58 |
| XD-52021 | 65 | 44.22 | 6.33 | 75.64 | 6.19 |
| XD-52022 | 66 | 22.50 | 2.34 | 58.69 | 9.81 |
| XD-52023 | 67 | 34.91 | 2.02 | 51.62 | 4.22 |
| XD-52024 | 68 | 19.96 | 2.38 | 35.07 | 4.09 |
| XD-52025 | 69 | 23.49 | 3.91 | 41.48 | 4.91 |
| XD-52026 | 88 | 17.12 | 1.10 | 40.66 | 0.93 |
| XD-52027 | 92 | 18.13 | 1.59 | 48.38 | 3.28 |
| XD-52030 | 97 | 28.95 | 1.42 | 53.78 | 1.53 |
| XD-52031 | 98 | 28.87 | 1.58 | 81.33 | 9.51 |
| XD-52032 | 99 | 36.20 | 1.06 | 40.59 | 6.69 |
| XD-52035 | 241 | 7.85 | 0.77 | 18.30 | 4.84 |
| XD-52038 | 245 | 19.45 | 2.73 | 30.77 | 5.16 |
| XD-52041 | 387 | 17.05 | 2.22 | 37.84 | 6.52 |
| XD-52042 | 406 | 11.66 | 1.34 | 28.40 | 4.62 |
| XD-52043 | 409 | 68.41 | 8.51 | 69.48 | 11.82 |
| XD-52044 | 412 | 12.17 | 1.32 | 24.94 | 1.87 |
| XD-52051 | 721 | 23.95 | 1.16 | 26.88 | 3.51 |
| XD-52054 | 871 | 53.07 | 4.34 | 69.78 | 8.54 |
| XD-52057 | 885 | 30.49 | 2.92 | 60.72 | 7.24 |
| XD-52058 | 888 | 31.55 | 4.87 | 33.67 | 3.28 |
| XD-52059 | 892 | 34.54 | 2.71 | 65.99 | 10.28 |
| XD-52060 | 895 | 26.97 | 4.38 | 41.67 | 6.43 |
| XD-52063 | 997 | 25.48 | 2.04 | 34.65 | 2.81 |
| XD-52065 | 1051 | 17.38 | 2.84 | 25.34 | 3.71 |
| XD-52066 | 1051 | 17.69 | 1.09 | 32.38 | 5.71 |
| XD-52067 | 1053 | 27.23 | 10.49 | 65.81 | 9.27 |
| XD-52068 | 1054 | 17.22 | 1.36 | 34.56 | 3.89 |
| XD-52070 | 1055 | 47.40 | 4.82 | 67.03 | 6.53 |
| XD-52079 | 1303 | 42.03 | 2.79 | 46.05 | 5.59 |
| XD-52084 | 1445 | 13.03 | 1.30 | 22.50 | 4.37 |
| XD-52085 | 1448 | 14.37 | 1.86 | 20.91 | 4.98 |
| XD-52089 | 1531 | 15.44 | 2.58 | 38.20 | 6.39 |
| XD-52090 | 1534 | 33.36 | 3.80 | 52.74 | 6.07 |
| XD-52091 | 1536 | 45.36 | 8.26 | 64.89 | 3.38 |
| XD-52092 | 1537 | 17.56 | 2.48 | 44.25 | 3.51 |
| XD-52093 | 1539 | 16.73 | 1.91 | 32.80 | 4.64 |
| XD-52094 | 1542 | 35.34 | 3.11 | 42.60 | 6.95 |
| XD-52095 | 1546 | 69.47 | 7.59 | 71.21 | 2.91 |
| XD-52096 | 1549 | 17.75 | 2.17 | 45.23 | 8.19 |
| XD-52099 | 1581 | 20.76 | 0.99 | 39.54 | 2.49 |
| XD-52100 | 1588 | 18.25 | 1.96 | 29.34 | 4.38 |
| XD-52105 | 1616 | 24.45 | 3.33 | 39.98 | 7.61 |
| XD-52106 | 1617 | 20.06 | 1.17 | 21.73 | 1.56 |
| XD-52107 | 1619 | 97.84 | 8.32 | 81.74 | 19.98 |
| XD-52108 | 1626 | 19.77 | 1.86 | 40.07 | 4.39 |
| XD-52109 | 1627 | 13.20 | 2.35 | 20.43 | 2.59 |
| XD-52115 | 1670 | 21.53 | 1.92 | 28.20 | 4.30 |
| XD-52116 | 1671 | 26.31 | 2.83 | 28.17 | 4.63 |
| XD-52120 | 1675 | 27.73 | 2.82 | 49.23 | 4.99 |
| XD-52125 | 1686 | 28.70 | 3.09 | 62.38 | 5.29 |
| XD-52130 | 1836 | 19.18 | 1.07 | 24.04 | 2.79 |
| XD-52131 | 1837 | 34.35 | 3.90 | 36.64 | 6.30 |
| XD-52134 | 1840 | 17.80 | 3.03 | 24.14 | 3.41 |
| XD-52135 | 1840 | 18.16 | 1.51 | 28.60 | 3.17 |
| XD-52137 | 1999 | 79.06 | 4.51 | 85.84 | 4.64 |
| XD-52139 | 2004 | 16.40 | 1.48 | 27.50 | 4.14 |
| XD-52145 | 2110 | 38.81 | 5.50 | 32.77 | 2.72 |
| XD-52155 | 2134 | 33.61 | 2.46 | 36.32 | 2.75 |
| XD-52156 | 2135 | 40.04 | 3.03 | 62.36 | 4.80 |
| XD-52157 | 2136 | 24.60 | 0.76 | 67.48 | 12.66 |
| XD-52158 | 2148 | 43.40 | 6.50 | 84.94 | 5.04 |
| XD-52159 | 2154 | 40.42 | 1.50 | 65.60 | 11.34 |
| XD-52160 | 2157 | 12.60 | 1.00 | 22.01 | 2.91 |
| XD-52161 | 2158 | 36.97 | 1.92 | 55.44 | 5.55 |
| XD-52162 | 2168 | 60.70 | 3.44 | 78.63 | 9.94 |
| XD-52163 | 2169 | 45.58 | 1.20 | 53.79 | 4.25 |
| XD-52164 | 2172 | 35.59 | 3.48 | 55.06 | 10.07 |
| XD-52166 | 2175 | 58.14 | 3.63 | 85.01 | 8.72 |
| XD-52167 | 2176 | 68.94 | 5.90 | 68.53 | 4.32 |
| XD-52168 | 2177 | 45.11 | 4.12 | 41.23 | 6.72 |
| XD-52169 | 2181 | 40.16 | 11.43 | 35.83 | 5.66 |
| XD-52170 | 2188 | 36.19 | 2.23 | 36.78 | 2.96 |
| XD-52173 | 2192 | 28.88 | 1.52 | 39.78 | 8.51 |
| XD-52174 | 2192 | 27.32 | 2.05 | 43.02 | 4.84 |
| XD-52175 | 2193 | 46.62 | 0.50 | 70.96 | 18.20 |
| XD-52179 | 2401 | 45.52 | 2.21 | 61.20 | 10.31 |
| XD-52180 | 2424 | 39.77 | 4.36 | 32.81 | 3.65 |
| XD-52181 | 2425 | 36.62 | 1.09 | 34.73 | 2.31 |
| XD-52184 | 2427 | 47.06 | 3.91 | 55.77 | 5.01 |
| XD-52185 | 2429 | 33.29 | 5.00 | 41.02 | 2.71 |
| XD-52186 | 2429 | 37.22 | 2.15 | 48.75 | 4.39 |
| XD-52187 | 2430 | 43.20 | 1.77 | 47.00 | 7.57 |
| XD-52188 | 2432 | 30.31 | 3.00 | 53.83 | 3.07 |
| XD-52189 | 2432 | 31.98 | 1.83 | 55.59 | 1.15 |
| XD-52190 | 2433 | 30.69 | 3.13 | 53.21 | 3.60 |
| XD-52191 | 2433 | 36.93 | 3.14 | 63.73 | 4.23 |
| XD-52194 | 2543 | 26.54 | 1.63 | 46.60 | 1.66 |
| XD-52195 | 2605 | 52.23 | 3.77 | 61.56 | 8.02 |
| XD-52197 | 2657 | 25.47 | 1.81 | 35.26 | 2.00 |
| XD-52198 | 2663 | 45.95 | 2.30 | 37.07 | 1.79 |
| XD-52199 | 2663 | 44.04 | 4.45 | 39.19 | 5.08 |
| XD-52200 | 2720 | 42.83 | 3.47 | 61.39 | 8.51 |
| XD-52202 | 2792 | 36.27 | 3.20 | 35.56 | 5.15 |
| XD-52203 | 2793 | 31.84 | 4.16 | 38.10 | 4.04 |
| XD-52204 | 2800 | 37.74 | 4.28 | 43.18 | 6.39 |
| XD-52205 | 2806 | 28.90 | 2.24 | 41.64 | 7.00 |

### Example 2. Dose Response Screening of the TFR2 siRNAs in HepG2 Cells

Several siRNAs displaying the best activity in the dual-dose screen were selected for more detailed dose response analysis. The experiment was performed essentially as described in Example 1. The siRNAs were used in 10 concentrations starting at 50 nM in 5-fold dilutions steps down to ˜40 pM. For defining the IC50 values, the dose-response curves were analyzed with XLit (Excel add-in) following a 4 parameter logistic fit model no. 205, fit=(A+((B−A)/(1+((C/x)\hat{ }D)))), where A is the bottom plateau of the curve (i.e., the final minimum y value, wherein y is the relative mRNA level), B is the top of the plateau of the curve(i.e., the final maximum y value), C is the IC50 value represents the x value at which the middle y value is attained, D is the slope factor, and x is concentration. The results of this analysis, showing IC50 and maximum knockdown effect are shown in Table 4.

TABLE 4
Results of the activity analysis of the initial set
of TFR2 siRNAs in HepG2 cells in dose response

| | | | Maximum |
|---|---|---|---|
| siRNA ID | Starting nt | IC50 (nM) | inhibition (%) |
| XD-52020 | 39 | 0.20 | 77.84 |
| XD-52024 | 68 | 0.09 | 75.56 |
| XD-52025 | 69 | 3.44 | 60.95 |
| XD-52026 | 88 | 0.64 | 73.44 |
| XD-52027 | 92 | 1.98 | 70.98 |
| XD-52035 | 241 | 0.05 | 88.58 |
| XD-52038 | 245 | 0.70 | 73.48 |
| XD-52041 | 387 | 0.27 | 77.09 |
| XD-52042 | 406 | 0.40 | 87.32 |
| XD-52044 | 412 | 0.40 | 84.67 |
| XD-52051 | 721 | 0.24 | 74.52 |
| XD-52060 | 895 | 0.65 | 69.31 |
| XD-52063 | 997 | 0.53 | 72.81 |
| XD-52065 | 1051 | 0.11 | 81.34 |
| XD-52066 | 1051 | 0.62 | 72.21 |
| XD-52068 | 1054 | 0.53 | 76.32 |
| XD-52084 | 1445 | 0.17 | 83.20 |
| XD-52085 | 1448 | 0.06 | 86.95 |
| XD-52089 | 1531 | 1.78 | 74.00 |
| XD-52092 | 1537 | 0.98 | 72.41 |
| XD-52093 | 1539 | 1.84 | 74.38 |
| XD-52099 | 1581 | 0.76 | 75.31 |
| XD-52100 | 1588 | 0.63 | 84.83 |
| XD-52105 | 1616 | 0.71 | 78.86 |
| XD-52106 | 1617 | 0.08 | 78.99 |
| XD-52108 | 1626 | 0.38 | 75.55 |
| XD-52109 | 1627 | 0.16 | 85.87 |
| XD-52115 | 1670 | 0.23 | 78.76 |
| XD-52116 | 1671 | 0.10 | 70.52 |
| XD-52130 | 1836 | 0.12 | 79.59 |
| XD-52134 | 1840 | 0.09 | 81.77 |
| XD-52135 | 1840 | 0.15 | 81.60 |
| XD-52139 | 2004 | 0.56 | 78.05 |
| XD-52160 | 2157 | 0.11 | 86.20 |
| XD-52173 | 2192 | 2.36 | 70.71 |
| XD-52174 | 2192 | 1.49 | 67.72 |
| XD-52197 | 2657 | 0.02 | 69.32 |
| XD-52205 | 2806 | 0.50 | 65.67 |

All siRNAs with sub-nanomolar IC50 values were analyzed for sequence specificity by screening RNA sequence databases, expression of potential off-target genes in hepatocytes and positions of the mismatches along siRNA. All siRNAs likely to have dangerous sequence-mediated off-target effects in hepatocytes were discarded.

### Example 3. Chemical Optimization of Selected TFR2 siRNAs

Several siRNAs starting from positions 241, 412, 1051, 1448, 1627, 1840, 2157, and 2657 showing the best activity in the dose response analysis in HepG2 cells and the best sequence specificity were selected for further chemical optimization and synthesized as generally described in Example 1 and as follows. It will be understood by those of skill in the art that alternative methodologies and materials for oligonucleotide synthesis are available and are encompassed by this disclosure.

Antisense strands were mostly synthesized on Universal linker loaded controlled pore glass (CPG) frit based-solid support, available from Biocomma (#DS0500, porosity of 500 Å) (except X169635 and X169639, which were assembled on 2′-OMe-C loaded CPG solid support available from LGC Biosearch Technologies (Petaluma, CA, USA) (#BG7-1030, porosity of 498 Å, 84 μmol/g loading). As noted, alternate synthesis methods can be used to synthesize the siRNAs in this example and that of Example 1. Sense strands were synthesized on the monomeric GalNAc-TEG immobilised CPG solid support sourced from Hongene Biotech, China (#ON-431, porosity of 500 Å, 49 μmol/g loading). Sense strands were synthesized on the monomeric GalNAc-TEG immobilised CPG solid support sourced from Hongene biotech, China (#ON-431, porosity of 500 Å, 49 μmol/g loading).

All 2′-modified RNA phosphoramidites as well as the majority of ancillary reagents (if not separately mentioned) for use in the current example as well as in Example 1 were purchased from SAFC, Proligo, Sigma Aldrich (Hamburg, Germany). Specifically, the following 2′-O-Methyl phosphoramidites were used: (5′-O-dimethoxytrityl-N6-(benzoyl)-2′-O-methyl-adenosine-3′-O-(2-cyanoethyl-N,N-diisopropylamino) phosphoramidite, 5′-O-dimethoxytrityl-N4-(acetyl)-2′-O-methyl-cytidine-3′-O-(2-cyanoethyl-N,N-diisopropylamino) phosphoramidite, (5′-O-dimethoxytrityl-N2-(isobutyryl)-2′-O-methyl-guanosine-3′-O-(2-cyanoethyl-N,N-diisopropylamino) phosphoramidite and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-TBDMS phosphoramidites carried the same protecting groups as the 2′-O-methyl RNA amidites. In addition to these, the other phosphoramidites were sourced as follows: 5′-vinyl phosphonate-2′-OMe-U (POM protected) (#LK2579), spacer C3 (#LK2113) and (invdT) (#LK2020) phosphoramidites were purchased from LGC Biosearch Technologies; monomeric GalNAc-TEG phosphoramidite (#OP-123) was procured from Hongene biotech, China; 2′-5′-linked 3′-deoxy phosphoramidites (3′-dA(bz)(#10-1004), 3′-dC(bz)(#10-1064), 3′-dG(dmf)(#10-1074) and 3′-dT(#10-1084)) were purchased from Glen Research; 2′-5′-linked 3′-OMe phosphoramidites (3′-OMe A(bz)(#ANP-2901), 3′-OMe C(bz)(#ANP-2902), 3′-OMe G(ibu)(#ANP-2903) and 3′-OMe U(#ANP-2904)) were purchased from ChemGenes; threose nucleic acid phosphoramidites (TNA-A(bz) (#STA-AM10063), TNA-C(ac) (#STA-AM10083), TNA-G(dmf) (#STA-AM10349) and TNA-U (#STA-AM10956)) were purchased from Wuxiapptech, China. Amidite solutions (50 mM) used were prepared from the amidites, as commercially sourced, in anhydrous acetonitrile and molecular sieves (3 Å, 8-12 mesh, Car Roth, #N893.1) were added.

5-Ethyl thiotetrazole (ETT, 500 mM in acetonitrile, 99.8%, Biosolve #0022112402BS) was used as activator solution. Coupling times were 6 minutes. In order to introduce phosphorothioate linkages a 100 mM solution of 3-Amino-1,2,4-dithiazole-5-thione (or Xanthane hydride obtained from TCI Chemicals, Germany, (Purity: >96.0% (HPLC) cas: 6846-35-1)) dissolved in ACN-pyridine (2:3 v/v) was employed as sulfurizing agent. The other ancillary reagents used were as follows: Iodine-Oxidizer (50 mM I₂ (in Pyridine-H₂O (9:1 v/v))), TCA (3% in DCM) for deblocking, Cap A (Acetic anhydride in THF (9.1:90.9 v/v)) and Cap B (THF, N-Methylimidazole and Pyridine (8:1:1 v/v/v) as Capping agents. Oligonucleotides were all synthesized with the removal of the final DMT protecting group (“DMT-Off”).

Cleavage De-Protection: After finalization of the solid phase synthesis, oligonucleotides were cleaved from the solid support by addition of AMA (1:1 (v/v) mixture of concentrated aqueous ammonia and 40% aqueous methylamine, both available from Sigma Aldrich) and collected in 96 well plates. To achieve quantitative removal of all the protecting groups the solutions were incubated with shaking at 33° C. Samples were thereafter dried under reduced pressure and reconstituted in 250 mM Tris(hydroxymethyl)aminomethane (TRIS) pH7 to yield crude sample solutions for subsequent purification.

Crude preparations were thereafter purified by Anion Exchange Chromatography using a Dionex DNA Pac100 (9×250 mm)-column (ThermoFisher, Dreieich, Germany) on an AKTA Purifier system (GE Healthcare, Freiburg, Germany) equipped with an auto-sampler (A905) and a fraction collector (Frac-950). Buffer A was 20 mM TRIS pH 7.4 and contained 20% acetonitrile and buffer B contained 500 mM sodium perchlorate in buffer A. Appropriate fractions were pooled and precipitated overnight in the freezer using 3M NaOAc, pH=5.2 and a mixture of ethanol and isopropanol. Pellets were reconstituted in purified water. The purified sense and antisense strands were quantified by measuring the UV absorption at 260 nm. Subsequently, the materials were assessed with respect to fulfillment of pre-agreed specifications for MS-identity (+/−0.05% of calculated mass (by ESI-MS)) and purity (single strand purity>85%, as per integration of the UV signal of the analytical AEX trace).

The purified complementary strands were then mixed in an equimolar ratio and dried in a SpeedVac concentrator. Subsequently, 300 μL of purified water were added to achieve a final duplex concentration of 100 μM. The duplex solutions were placed into a water bath at 70° C., which was subsequently cooled to RT within 3 h. The resultant siRNA-duplexes were characterized by Size-exclusion chromatography (SEC) towards fulfillment of pre agreed specification of Purity (Duplex purity>90%, as per integration of the UV signal of the analytical SEC trace).

Using the described methods, the siRNAs were synthesized in 13 different chemical modification patterns each and compared to the original siRNAs used in the initial screening in HepG2 cells. Nine of these chemical modification patterns contained a chemical modification (a 2′-5′ bridge between positions 7 and 8 of the antisense strand with 3′H or 3′OMe in position 7, or a TNA nucleotide in position 7) reported to reduce seed-mediated off-target effects of a given siRNA (see for example U.S. Pat. No. 10,421,962). The list of these siRNAs, as well as SEQ ID NO: 308, claimed herein, is shown in Table 5.

TABLE 5
Chemical optimization of modified TFR2 siRNAs

| SEQ | | | |
|---|---|---|---|
| ID | Starting | | Sense strand |
| NO | nt | siRNA ID | Antisense strand |
| 193 | 241 | XD-65715 | 5′-(C3)csasggCfaGfCfCfaaaccucauasLsLsL-3′ |
| 194 | | | 5′-(vinu)sAfsugaGfgUfUfuggcUfgCfcugsgsg-3′ |
| 193 | 241 | XD-65716 | 5′-(C3)csasggCfaGfCfCfaaaccucauasLsLsL-3′ |
| 195 | | | 5′-(vinu)sAfsugaGf(3dG)UfUfuggcUfgCfcugsgsg-3′ |
| 193 | 241 | XD-65717 | 5′-(C3)csasggCfaGfCfCfaaaccucauasLsLsL-3′ |
| 196 | | | 5′-(vinu)sAfsugaGf(2-5g)UfUfuggcUfgCfcugsgsg-3′ |
| 193 | 241 | XD-65718 | 5′-(C3)csasggCfaGfCfCfaaaccucauasLsLsL-3′ |
| 197 | | | 5′-(vinu)sAfsugaGfGtUfUfuggcUfgCfcugsgsg-3′ |
| 193 | 241 | XD-65719 | 5′-(C3)csasggCfaGfCfCfaaaccucauasLsLsL-3′ |
| 198 | | | 5′-(vinu)sAfsugaGfguUfuggcUfgCfcugsgsg-3′ |
| 193 | 241 | XD-65720 | 5′-(C3)csasggCfaGfCfCfaaaccucauasLsLsL-3′ |
| 199 | | | 5′-(vinu)sAfsugaGf(3dG)uUfuggcUfgCfcugsgsg-3′ |
| 193 | 241 | XD-65721 | 5′-(C3)csasggCfaGfCfCfaaaccucauasLsLsL-3′ |
| 200 | | | 5′-(vinu)sAfsugaGf(2-5g)uUfuggcUfgCfcugsgsg-3′ |
| 193 | 241 | XD-65722 | 5′-(C3)csasggCfaGfCfCfaaaccucauasLsLsL-3′ |
| 201 | | | 5′-(vinu)sAfsugaGfGtuUfuggcUfgCfcugsgsg-3′ |
| 193 | 241 | XD-65723 | 5′-(C3)csasggCfaGfCfCfaaaccucauasLsLsL-3′ |
| 202 | | | 5′-(vinu)sAfsugagguuuggcUfgCfcugsgsg-3′ |
| 193 | 241 | XD-65724 | 5′-(C3)csasggCfaGfCfCfaaaccucauasLsLsL-3′ |
| 203 | | | 5′-(vinu)sAfsugag(3dG)uuuggcUfgCfcugsgsg-3′ |
| 193 | 241 | XD-65725 | 5′-(C3)csasggCfaGfCfCfaaaccucauasLsLsL-3′ |
| 204 | | | 5′-(vinu)sAfsugag(2-5g)uuuggcUfgCfcugsgsg-3′ |
| 193 | 241 | XD-65726 | 5′-(C3)csasggCfaGfCfCfaaaccucauasLsLsL-3′ |
| 205 | | | 5′-(vinu)sAfsugagGtuuuggcUfgCfcugsgsg-3′ |
| 309 | 241 | XD-65727 | 5′-(C3)csasGfgcaGfCfCfaaAfccucAfuasLsLsL-3′ |
| 206 | | | 5′-(vinu)sAfsugagguuuggcUfgccugsgsg-3′ |
| 207 | 412 | XD-65728 | 5′-(C3)csusauGfaGfCfCfugaccuggaasLsLsL-3′ |
| 208 | | | 5′-(vinu)sUfsccaGfgUfCfaggcUfcAfuagsusu-3′ |
| 207 | 412 | XD-65729 | 5′-(C3)csusauGfaGfCfCfugaccuggaasLsLsL-3′ |
| 209 | | | 5′-(vinu)sUfsccaGf(3dG)UfCfaggcUfcAfuagsusu-3′ |
| 207 | 412 | XD-65730 | 5′-(C3)csusauGfaGfCfCfugaccuggaasLsLsL-3′ |
| 210 | | | 5′-(vinu)sUfsccaGf(2-5g)UfCfaggcUfcAfuagsusu-3′ |
| 207 | 412 | XD-65731 | 5′-(C3)csusauGfaGfCfCfugaccuggaasLsLsL-3′ |
| 211 | | | 5′-(vinu)sUfsccaGfGtUfCfaggcUfcAfuagsusu-3′ |
| 207 | 412 | XD-65732 | 5′-(C3)csusauGfaGfCfCfugaccuggaasLsLsL-3′ |
| 212 | | | 5′-(vinu)sUfsccaGfguCfaggcUfcAfuagsusu-3′ |
| 207 | 412 | XD-65733 | 5′-(C3)csusauGfaGfCfCfugaccuggaasLsLsL-3′ |
| 213 | | | 5′-(vinu)sUfsccaGf(3dG)uCfaggcUfcAfuagsusu-3′ |
| 207 | 412 | XD-65734 | 5′-(C3)csusauGfaGfCfCfugaccuggaasLsLsL-3′ |
| 214 | | | 5′-(vinu)sUfsccaGf(2-5g)uCfaggcUfcAfuagsusu-3′ |
| 207 | 412 | XD-65735 | 5′-(C3)csusauGfaGfCfCfugaccuggaasLsLsL-3′ |
| 215 | | | 5′-(vinu)sUfsccaGfGtuCfaggcUfcAfuagsusu-3′ |
| 207 | 412 | XD-65736 | 5′-(C3)csusauGfaGfCfCfugaccuggaasLsLsL-3′ |
| 216 | | | 5′-(vinu)sUfsccaggucaggcUfcAfuagsusu-3′ |
| 207 | 412 | XD-65737 | 5′-(C3)csusauGfaGfCfCfugaccuggaasLsLsL-3′ |
| 217 | | | 5′-(vinu)sUfsccag(3dG)ucaggcUfcAfuagsusu-3′ |
| 207 | 412 | XD-65738 | 5′-(C3)csusauGfaGfCfCfugaccuggaasLsLsL-3′ |
| 218 | | | 5′-(vinu)sUfsccag(2-5g)ucaggcUfcAfuagsusu-3′ |
| 207 | 412 | XD-65739 | 5′-(C3)csusauGfaGfCfCfugaccuggaasLsLsL-3′ |
| 219 | | | 5′-(vinu)sUfsccagGtucaggcUfcAfuagsusu-3′ |
| 310 | 412 | XD-65740 | 5′-(C3)csusAfugaGfCfCfugAfccugGfaasLsLsL-3′ |
| 220 | | | 5′-(vinu)sUfsccaggucaggcUfcauagsusu-3′ |
| 221 | 1051 | XD-65741 | 5′-(C3)ususccUfuCfAfAfucaaacccaasLsLsL-3′ |
| 222 | | | 5′-(vinu)sUfsgggUfuUfGfauugAfaGfgaasgsg-3′ |
| 221 | 1051 | XD-65742 | 5′-(C3)ususccUfuCfAfAfucaaacccaasLsLsL-3′ |
| 223 | | | 5′-(vinu)sUfsgggUf(3dU)UfGfauugAfaGfgaasgsg-3′ |
| 221 | 1051 | XD-65743 | 5′-(C3)ususccUfuCfAfAfucaaacccaasLsLsL-3′ |
| 224 | | | 5′-(vinu)sUfsgggUf(2-5u)UfGfauugAfaGfgaasgsg-3′ |
| 221 | 1051 | XD-65744 | 5′-(C3)ususccUfuCfAfAfucaaacccaasLsLsL-3′ |
| 225 | | | 5′-(vinu)sUfsgggUfUtUfGfauugAfaGfgaasgsg-3′ |
| 221 | 1051 | XD-65745 | 5′-(C3)ususccUfuCfAfAfucaaacccaasLsLsL-3′ |
| 226 | | | 5′-(vinu)sUfsgggUfuuGfauugAfaGfgaasgsg-3′ |
| 221 | 1051 | XD-65746 | 5′-(C3)ususccUfuCfAfAfucaaacccaasLsLsL-3′ |
| 227 | | | 5′-(vinu)sUfsgggUf(3dU)uGfauugAfaGfgaasgsg-3′ |
| 221 | 1051 | XD-65747 | 5′-(C3)ususccUfuCfAfAfucaaacccaasLsLsL-3′ |
| 228 | | | 5′-(vinu)sUfsgggUf(2-5u)uGfauugAfaGfgaasgsg-3′ |
| 221 | 1051 | XD-65748 | 5′-(C3)ususccUfuCfAfAfucaaacccaasLsLsL-3′ |
| 229 | | | 5′-(vinu)sUfsgggUfUtuGfauugAfaGfgaasgsg-3′ |
| 221 | 1051 | XD-65749 | 5′-(C3)ususccUfuCfAfAfucaaacccaasLsLsL-3′ |
| 230 | | | 5′-(vinu)sUfsggguuugauugAfaGfgaasgsg-3′ |
| 221 | 1051 | XD-65750 | 5′-(C3)ususccUfuCfAfAfucaaacccaasLsLsL-3′ |
| 231 | | | 5′-(vinu)sUfsgggu(3dU)ugauugAfaGfgaasgsg-3′ |
| 221 | 1051 | XD-65751 | 5′-(C3)ususccUfuCfAfAfucaaacccaasLsLsL-3′ |
| 232 | | | 5′-(vinu)sUfsgggu(2-5u)ugauugAfaGfgaasgsg-3′ |
| 221 | 1051 | XD-65752 | 5′-(C3)ususccUfuCfAfAfucaaacccaasLsLsL-3′ |
| 233 | | | 5′-(vinu)sUfsggguUtugauugAfaGfgaasgsg-3′ |
| 311 | 1051 | XD-65753 | 5′-(C3)ususCfcuuCfAfAfucAfaaccCfaasLsLsL-3′ |
| 234 | | | 5′-(vinu)sUfsggguuugauugAfaggaasgsg-3′ |
| 235 | 1448 | XD-65754 | 5′-(C3)asgsaaGfuCfUfCfcucuucaucasLsLsL-3′ |
| 236 | | | 5′-(vinu)sGfsaugAfaGfAfggagAfcUfucusgsc-3′ |
| 235 | 1448 | XD-65755 | 5′-(C3)asgsaaGfuCfUfCfcucuucaucasLsLsL-3′ |
| 237 | | | 5′-(vinu)sGfsaugAf(3dA)GfAfggagAfcUfucusgsc-3′ |
| 235 | 1448 | XD-65756 | 5′-(C3)asgsaaGfuCfUfCfcucuucaucasLsLsL-3′ |
| 238 | | | 5′-(vinu)sGfsaugAf(2-5a)GfAfggagAfcUfucusgsc-3′ |
| 235 | 1448 | XD-65757 | 5′-(C3)asgsaaGfuCfUfCfcucuucaucasLsLsL-3′ |
| 239 | | | 5′-(vinu)sGfsaugAfAtGfAfggagAfcUfucusgsc-3′ |
| 235 | 1448 | XD-65758 | 5′-(C3)asgsaaGfuCfUfCfcucuucaucasLsLsL-3′ |
| 240 | | | 5′-(vinu)sGfsaugAfagAfggagAfcUfucusgsc-3′ |
| 235 | 1448 | XD-65759 | 5′-(C3)asgsaaGfuCfUfCfcucuucaucasLsLsL-3′ |
| 241 | | | 5′-(vinu)sGfsaugAf(3dA)gAfggagAfcUfucusgsc-3′ |
| 235 | 1448 | XD-65760 | 5′-(C3)asgsaaGfuCfUfCfcucuucaucasLsLsL-3′ |
| 242 | | | 5′-(vinu)sGfsaugAf(2-5a)gAfggagAfcUfucusgsc-3′ |
| 235 | 1448 | XD-65761 | 5′-(C3)asgsaaGfuCfUfCfcucuucaucasLsLsL-3′ |
| 243 | | | 5′-(vinu)sGfsaugAfAtgAfggagAfcUfucusgsc-3′ |
| 235 | 1448 | XD-65762 | 5′-(C3)asgsaaGfuCfUfCfcucuucaucasLsLsL-3′ |
| 244 | | | 5′-(vinu)sGfsaugaagaggagAfcUfucusgsc-3′ |
| 235 | 1448 | XD-65763 | 5′-(C3)asgsaaGfuCfUfCfcucuucaucasLsLsL-3′ |
| 245 | | | 5′-(vinu)sGfsauga(3dA)gaggagAfcUfucusgsc-3′ |
| 235 | 1448 | XD-65764 | 5′-(C3)asgsaaGfuCfUfCfcucuucaucasLsLsL-3′ |
| 246 | | | 5′-(vinu)sGfsauga(2-5a)gaggagAfcUfucusgsc-3′ |
| 235 | 1448 | XD-65765 | 5′-(C3)asgsaaGfuCfUfCfcucuucaucasLsLsL-3′ |
| 247 | | | 5′-(vinu)sGfsaugaAtgaggagAfcUfucusgsc-3′ |
| 312 | 1448 | XD-65766 | 5′-(C3)asgsAfaguCfUfCfcuCfuucaUfcasLsLsL-3′ |
| 248 | | | 5′-(vinu)sGfsaugaagaggagAfcuucusgsc-3′ |
| 249 | 1627 | XD-65767 | 5′-(C3)csasuuGfaGfAfGfuguccugaaasLsLsL-3′ |
| 250 | | | 5′-(vinu)sUfsucaGfgAfCfacucUfcAfaugsasg-3′ |
| 249 | 1627 | XD-65768 | 5′-(C3)csasuuGfaGfAfGfuguccugaaasLsLsL-3′ |
| 251 | | | 5′-(vinu)sUfsucaGf(3dG)AfCfacucUfcAfaugsasg-3′ |
| 249 | 1627 | XD-65769 | 5′-(C3)csasuuGfaGfAfGfuguccugaaasLsLsL-3′ |
| 252 | | | 5′-(vinu)sUfsucaGf(2-5g)AfCfacucUfcAfaugsasg-3′ |
| 249 | 1627 | XD-65770 | 5′-(C3)csasuuGfaGfAfGfuguccugaaasLsLsL-3′ |
| 253 | | | 5′-(vinu)sUfsucaGfGtAfCfacucUfcAfaugsasg-3′ |
| 249 | 1627 | XD-65771 | 5′-(C3)csasuuGfaGfAfGfuguccugaaasLsLsL-3′ |
| 254 | | | 5′-(vinu)sUfsucaGfgaCfacucUfcAfaugsasg-3′ |
| 249 | 1627 | XD-65772 | 5′-(C3)csasuuGfaGfAfGfuguccugaaasLsLsL-3′ |
| 255 | | | 5′-(vinu)sUfsucaGf(3dG)aCfacucUfcAfaugsasg-3′ |
| 249 | 1627 | XD-65773 | 5′-(C3)csasuuGfaGfAfGfuguccugaaasLsLsL-3′ |
| 256 | | | 5′-(vinu)sUfsucaGf(2-5g)aCfacucUfcAfaugsasg-3′ |
| 249 | 1627 | XD-65774 | 5′-(C3)csasuuGfaGfAfGfuguccugaaasLsLsL-3′ |
| 257 | | | 5′-(vinu)sUfsucaGfGtaCfacucUfcAfaugsasg-3′ |
| 249 | 1627 | XD-65775 | 5′-(C3)csasuuGfaGfAfGfuguccugaaasLsLsL-3′ |
| 258 | | | 5′-(vinu)sUfsucaggacacucUfcAfaugsasg-3′ |
| 249 | 1627 | XD-65776 | 5′-(C3)csasuuGfaGfAfGfuguccugaaasLsLsL-3′ |
| 259 | | | 5′-(vinu)sUfsucag(3dG)acacucUfcAfaugsasg-3′ |
| 249 | 1627 | XD-65777 | 5′-(C3)csasuuGfaGfAfGfuguccugaaasLsLsL-3′ |
| 260 | | | 5′-(vinu)sUfsucag(2-5g)acacucUfcAfaugsasg-3′ |
| 249 | 1627 | XD-65778 | 5′-(C3)csasuuGfaGfAfGfuguccugaaasLsLsL-3′ |
| 261 | | | 5′-(vinu)sUfsucagGtacacucUfcAfaugsasg-3′ |
| 313 | 1627 | XD-65779 | 5′-(C3)csasUfugaGfAfGfugUfccugAfaasLsLsL-3′ |
| 262 | | | 5′-(vinu)sUfsucaggacacucUfcaaugsasg-3′ |
| 263 | 1840 | XD-65780 | 5′-(C3)asasagGfaGfGfAfcacuuaugaasLsLsL-3′ |
| 264 | | | 5′-(vinu)sUfscauAfaGfUfguccUfcCfuuusgsu-3′ |
| 263 | 1840 | XD-65781 | 5′-(C3)asasagGfaGfGfAfcacuuaugaasLsLsL-3′ |
| 265 | | | 5′-(vinu)sUfscauAf(3dA)GfUfguccUfcCfuuusgsu-3′ |
| 263 | 1840 | XD-65782 | 5′-(C3)asasagGfaGfGfAfcacuuaugaasLsLsL-3′ |
| 266 | | | 5′-(vinu)sUfscauAf(2-5a)GfUfguccUfcCfuuusgsu-3′ |
| 263 | 1840 | XD-65783 | 5′-(C3)asasagGfaGfGfAfcacuuaugaasLsLsL-3′ |
| 267 | | | 5′-(vinu)sUfscauAfAtGfUfguccUfcCfuuusgsu-3′ |
| 263 | 1840 | XD-65784 | 5′-(C3)asasagGfaGfGfAfcacuuaugaasLsLsL-3′ |
| 268 | | | 5′-(vinu)sUfscauAfagUfguccUfcCfuuusgsu-3′ |
| 263 | 1840 | XD-65785 | 5′-(C3)asasagGfaGfGfAfcacuuaugaasLsLsL-3′ |
| 269 | | | 5′-(vinu)sUfscauAf(3dA)gUfguccUfcCfuuusgsu-3′ |
| 263 | 1840 | XD-65786 | 5′-(C3)asasagGfaGfGfAfcacuuaugaasLsLsL-3′ |
| 270 | | | 5′-(vinu)sUfscauAf(2-5a)gUfguccUfcCfuuusgsu-3′ |
| 263 | 1840 | XD-65787 | 5′-(C3)asasagGfaGfGfAfcacuuaugaasLsLsL-3′ |
| 271 | | | 5′-(vinu)sUfscauAfAtgUfguccUfcCfuuusgsu-3′ |
| 263 | 1840 | XD-65788 | 5′-(C3)asasagGfaGfGfAfcacuuaugaasLsLsL-3′ |
| 272 | | | 5′-(vinu)sUfscauaaguguccUfcCfuuusgsu-3′ |
| 263 | 1840 | XD-65789 | 5′-(C3)asasagGfaGfGfAfcacuuaugaasLsLsL-3′ |
| 273 | | | 5′-(vinu)sUfscaua(3dA)guguccUfcCfuuusgsu-3′ |
| 263 | 1840 | XD-65790 | 5′-(C3)asasagGfaGfGfAfcacuuaugaasLsLsL-3′ |
| 274 | | | 5′-(vinu)sUfscaua(2-5a)guguccUfcCfuuusgsu-3′ |
| 263 | 1840 | XD-65791 | 5′-(C3)asasagGfaGfGfAfcacuuaugaasLsLsL-3′ |
| 275 | | | 5′-(vinu)sUfscauaAtguguccUfcCfuuusgsu-3′ |
| 314 | 1840 | XD-65792 | 5′-(C3)asasAfggaGfGfAfcaCfuuauGfaasLsLsL-3′ |
| 276 | | | 5′-(vinu)sUfscauaaguguccUfccuuusgsu-3′ |
| 277 | 2157 | XD-65793 | 5′-(C3)usgsuaCfaAfCfGfugcgcauaaasLsLsL-3′ |
| 278 | | | 5′-(vinu)sUfsuauGfcGfCfacguUfgUfacasusg-3′ |
| 277 | 2157 | XD-65794 | 5′-(C3)usgsuaCfaAfCfGfugcgcauaaasLsLsL-3′ |
| 279 | | | 5′-(vinu)sUfsuauGf(3dC)GfCfacguUfgUfacasusg-3′ |
| 277 | 2157 | XD-65795 | 5′-(C3)usgsuaCfaAfCfGfugcgcauaaasLsLsL-3′ |
| 280 | | | 5′-(vinu)sUfsuauGf(2-5c)GfCfacguUfgUfacasusg-3′ |
| 277 | 2157 | XD-65796 | 5′-(C3)usgsuaCfaAfCfGfugcgcauaaasLsLsL-3′ |
| 281 | | | 5′-(vinu)sUfsuauGfCtGfCfacguUfgUfacasusg-3′ |
| 277 | 2157 | XD-65797 | 5′-(C3)usgsuaCfaAfCfGfugcgcauaaasLsLsL-3′ |
| 282 | | | 5′-(vinu)sUfsuauGfcgCfacguUfgUfacasusg-3′ |
| 277 | 2157 | XD-65798 | 5′-(C3)usgsuaCfaAfCfGfugcgcauaaasLsLsL-3′ |
| 283 | | | 5′-(vinu)sUfsuauGf(3dC)gCfacguUfgUfacasusg-3′ |
| 277 | 2157 | XD-65799 | 5′-(C3)usgsuaCfaAfCfGfugcgcauaaasLsLsL-3′ |
| 284 | | | 5′-(vinu)sUfsuauGf(2-5c)gCfacguUfgUfacasusg-3′ |
| 277 | 2157 | XD-65800 | 5′-(C3)usgsuaCfaAfCfGfugcgcauaaasLsLsL-3′ |
| 285 | | | 5′-(vinu)sUfsuauGfCtgCfacguUfgUfacasusg-3′ |
| 277 | 2157 | XD-65801 | 5′-(C3)usgsuaCfaAfCfGfugcgcauaaasLsLsL-3′ |
| 286 | | | 5′-(vinu)sUfsuaugcgcacguUfgUfacasusg-3′ |
| 277 | 2157 | XD-65802 | 5′-(C3)usgsuaCfaAfCfGfugcgcauaaasLsLsL-3′ |
| 287 | | | 5′-(vinu)sUfsuaug(3dC)gcacguUfgUfacasusg-3′ |
| 277 | 2157 | XD-65803 | 5′-(C3)usgsuaCfaAfCfGfugcgcauaaasLsLsL-3′ |
| 288 | | | 5′-(vinu)sUfsuaug(2-5c)gcacguUfgUfacasusg-3′ |
| 277 | 2157 | XD-65804 | 5′-(C3)usgsuaCfaAfCfGfugcgcauaaasLsLsL-3′ |
| 289 | | | 5′-(vinu)sUfsuaugCtgcacguUfgUfacasusg-3′ |
| 315 | 2157 | XD-65805 | 5′-(C3)usgsUfacaAfCfGfugCfgcauAfaasLsLsL-3′ |
| 290 | | | 5′-(vinu)sUfsuaugcgcacguUfguacasusg-3′ |
| 291 | 2657 | XD-65806 | 5′-(C3)csgsguCfaUfAfCfugucgguuaasLsLsL-3′ |
| 292 | | | 5′-(vinu)sUfsaacCfgAfCfaguaUfgAfccgsusc-3′ |
| 291 | 2657 | XD-65807 | 5′-(C3)csgsguCfaUfAfCfugucgguuaasLsLsL-3′ |
| 293 | | | 5′-(vinu)sUfsaacCf(3dG)AfCfaguaUfgAfccgsusc-3′ |
| 291 | 2657 | XD-65808 | 5′-(C3)csgsguCfaUfAfCfugucgguuaasLsLsL-3′ |
| 294 | | | 5′-(vinu)sUfsaacCf(2-5g)AfCfaguaUfgAfccgsusc-3′ |
| 291 | 2657 | XD-65809 | 5′-(C3)csgsguCfaUfAfCfugucgguuaasLsLsL-3′ |
| 295 | | | 5′-(vinu)sUfsaacCfGtAfCfaguaUfgAfccgsusc-3′ |
| 291 | 2657 | XD-65810 | 5′-(C3)csgsguCfaUfAfCfugucgguuaasLsLsL-3′ |
| 296 | | | 5′-(vinu)sUfsaacCfgaCfaguaUfgAfccgsusc-3′ |
| 291 | 2657 | XD-65811 | 5′-(C3)csgsguCfaUfAfCfugucgguuaasLsLsL-3′ |
| 297 | | | 5′-(vinu)sUfsaacCf(3dG)aCfaguaUfgAfccgsusc-3′ |
| 291 | 2657 | XD-65812 | 5′-(C3)csgsguCfaUfAfCfugucgguuaasLsLsL-3′ |
| 298 | | | 5′-(vinu)sUfsaacCf(2-5g)aCfaguaUfgAfccgsusc-3′ |
| 291 | 2657 | XD-65813 | 5′-(C3)csgsguCfaUfAfCfugucgguuaasLsLsL-3′ |
| 299 | | | 5′-(vinu)sUfsaacCfGtaCfaguaUfgAfccgsusc-3′ |
| 291 | 2657 | XD-65814 | 5′-(C3)csgsguCfaUfAfCfugucgguuaasLsLsL-3′ |
| 300 | | | 5′-(vinu)sUfsaaccgacaguaUfgAfccgsusc-3′ |
| 291 | 2657 | XD-65815 | 5′-(C3)csgsguCfaUfAfCfugucgguuaasLsLsL-3′ |
| 301 | | | 5′-(vinu)sUfsaacc(3dG)acaguaUfgAfccgsusc-3′ |
| 291 | 2657 | XD-65816 | 5′-(C3)csgsguCfaUfAfCfugucgguuaasLsLsL-3′ |
| 302 | | | 5′-(vinu)sUfsaacc(2-5g)acaguaUfgAfccgsusc-3′ |
| 291 | 2657 | XD-65817 | 5′-(C3)csgsguCfaUfAfCfugucgguuaasLsLsL-3′ |
| 303 | | | 5′-(vinu)sUfsaaccGtacaguaUfgAfccgsusc-3′ |
| 316 | 2657 | XD-65818 | 5′-(C3)csgsGfucaUfAfCfugUfcgguUfaasLsLsL-3′ |
| 304 | | | 5′-(vinu)sUfsaaccgacaguaUfgaccgsusc-3′ |
| 308 | 2657 | | (C3)csgsguCfaUfAfCfugucgguuaa (sense strand only) |

As also described in Table 1 above, the sequence nomenclature in Table 5 is as follows: Nf: 2′-Fluoro (2′F) nucleotides; n: 2′-O-Methyl (2′ OMe) nucleotides; (2-Sn): 2′-S′ 3′-OMe nucleotides; (3dN): 2′-S′ 3′-H nucleotides; (vinu): 5′ vinyl-phosphonate 2′-OMe U nucleotide; Nt: TNA nucleotides; (C3): propanol or C3 alkyl moiety connected to the 5′ end of the strand via a phosphodiester linkage; s: phosphorothioate (Ps) backbone modification between adjacent nucleotides; L: Monomer of N-acetylgalactosamine (GalNAc) with a triethyleneglycol (TEG) linker.

### Example 4. Analysis of Chemically Optimized siRNAs for Attenuation of Seed-Mediated Effects in a Dual-Luciferase Assay

It is known that siRNAs can elicit seed-mediated miRNA-like off-target activity (see for example Suter et al., Org. Biomed. Chem. 15: 10029-10036, 2017). The siRNAs listed in Table 5 were assayed for the such seed-mediated off-target effects in a dual-luciferase assay.

A set of constructs in the psiCHECK vector (Promega) was generated. One set of psiCHECK plasmids contained complete 19-mer target sequences of each siRNA of interest for monitoring the on-target activity of the siRNAs. Another set of psiCHECK plasmids contained the 3× concatomer seed target sequences (3 copies of nucleotide sequences complementary to positions 2-8 of the antisense strands starting from their 5′ ends) of each siRNA to monitor seed-mediated activity of the siRNAs. Each of the target sequences (a completely matched or 3×-seed-matched) was individually cloned into the multiple cloning sites within the Renilla Luciferase (R-Luc) 3′ untranslated region of the psiCHECK vectors for monitoring on-target or seed-mediated off-target siRNA activity by measuring the R-Luc activity whereas the luminescence signal elicited by a Firefly Luciferase (F-Luc) expressed from the same plasmid but from a different promoter served for effect normalization.

The assay was performed in Hepa 1-6 cells co-transfected with individual psiCHECK plasmids (a constant concentration of 50 ng/well) and the corresponding siRNAs added in 5 concentrations from 20 nM to 2 pM in 10-fold dilutions steps. Cells transfected with the empty plasmid served as negative control. The transfections were performed in quadruplicates using Lipofectamine2000 (Invitrogen/Life Technologies). After 24 hours of incubation, DualGlo Assay (Promega) was performed according to the manufacturer's protocol. For each well, the R-Luc signal was normalized to the respective F-Luc signal to correct for plasmid loading. The activity of a given siRNA was expressed as percent of relative R-Luc activity (normalized to F-Luc) in treated cells, relative to the negative control signals (R-Luc normalized to F-Luc) averaged across negative control wells. IC20, IC50 and IC80 concentrations were calculated from the dose response curves using XLfit graph (MS Excel Add On) and the results are summarized in Tables 6A and 6B. “#N/A” designates “non-applicable” for cases when IC20/IC50/IC80 values could not be calculated.

As shown in Table 6A, in the majority of cases, varying chemical modification patterns compared to the one used in the initial screening in HepG2 cells did not alter the on-target maximal knockdown potency—albeit with few exceptions. For example, for siRNAs starting from position 241, the maximum on-target knockdown effects of all novel modification patterns were improved from 94% (XD-65727) to 95-98.5%. For siRNAs starting from position 1840, the maximum on target KD effect was improved from 91.5% (XD-65792) to 94-97% for all chemical modification variants but one (XD-65790) where it was reduced to 78.7%. Notably, this chemical modification pattern did not negatively affect the maximal KD by other assayed siRNAs.

As for the on-target IC50 values (Table 6A), the effects of various chemical modification patterns seemed to be mostly siRNA sequence-dependent. For example, for siRNAs starting from position 2657, all novel chemical modification patterns improved IC50 values (albeit to different extents) compared to the original siRNA (XD-65818). At the same time, for other original siRNAs, application of certain novel chemical modification patterns resulted in on-target IC50 worsening, e.g., compare XD65793 vs XD65805 (original) and XD65790 vs XD65792 (original) for siRNAs starting from positions 2157 and 1840, respectively. Notably, chemical modification patterns for XD65793 and XD65790 were different.

Testing siRNA KD activities based only their seed region recognition (modeling of seed-mediated off-target effects) (Table 6B) has demonstrated that this type of activity was present in all siRNAs which chemical modification patterns did not include a specifically modified position 7 of the antisense strand. Seed-mediated knockdown potency of these siRNAs strongly depended on their sequence and, to a lesser extent, on the type of chemical modification pattern (that did not involve specific modifications in the antisense position 7). Thus, siRNAs starting from position 1840 could induce only a 30-35% seed-mediated KD, whereas for siRNAs starting from positions 241 and 412 it was 40-50%, for siRNAs starting from position 1051-60-70%, for siRNA starting from positions 1448, 1627, 2657-70-80%, and for siRNA starting from position 2157->90%.

In the majority of cases, addition of specific chemical modifications in position 7 of the antisense strand resulted in reduction of seed-mediated KD potency (Table 6B). However, this effect was sequence- and modification type-dependent—not working equally well for all siRNAs. For example, for siRNAs starting from position 2157, none of the antisense position 7 modification types was able to reduce seed-mediated KD activity. At the same time, for siRNAs starting at position 241, all types of modifications at the antisense position 7 have completely abolished the seed-mediated KD. For siRNAs starting from positions 1051 and 1448, having 2′-5′ linkage between positions 7 and 8 of their antisense strands with position 7 being modified with 3′H resulted in a minimal reduction of seed-mediated KD whereas a substitution of 3′H with 3′Ome has practically eliminated the seed-mediated KD effect. In contrast, for siRNAs starting from position 2657, both 3′H and 3′Ome in antisense position 7 had similar profound effects in inhibiting seed-mediated KD.

Several siRNAs that demonstrated strong on-target knock down activity and little to no seed-mediated knockdown activity were selected for further analysis in primary human hepatocytes with GalNAc-mediated delivery.

TABLE 6A
Summary of on-target TFR2 siRNA activity

| | | Full length target sequence | | | |
|---|---|---|---|---|---|
| Starting | | | IC50 | IC80 | Maximum |
| position | siRNA ID | IC20 (nM) | (nM) | (nM) | KD (%) |
| 241 | XD-65715 | 0.0018 | 0.0083 | 0.0402 | 97.52 |
| | XD-65716 | 0.0018 | 0.0074 | 0.0318 | 96.88 |
| | XD-65717 | 0.0009 | 0.0036 | 0.0156 | 96.91 |
| | XD-65718 | 0.0013 | 0.0049 | 0.0182 | 98.31 |
| | XD-65719 | 0.0017 | 0.0089 | 0.0488 | 97.61 |
| | XD-65720 | 0.0019 | 0.0080 | 0.0352 | 96.91 |
| | XD-65721 | 0.0010 | 0.0042 | 0.0187 | 97.17 |
| | XD-65722 | 0.0015 | 0.0059 | 0.0232 | 98.54 |
| | XD-65723 | 0.0017 | 0.0088 | 0.0479 | 97.28 |
| | XD-65724 | 0.0048 | 0.0166 | 0.0641 | 95.03 |
| | XD-65725 | 0.0011 | 0.0049 | 0.0225 | 97.05 |
| | XD-65726 | 0.0018 | 0.0067 | 0.0252 | 98.06 |
| | XD-65727 | 0.0035 | 0.0145 | 0.0678 | 93.92 |
| 412 | XD-65728 | 0.0037 | 0.0173 | 0.0872 | 97.26 |
| | XD-65729 | 0.0024 | 0.0100 | 0.0441 | 97.50 |
| | XD-65730 | 0.0016 | 0.0068 | 0.0288 | 98.08 |
| | XD-65731 | 0.0018 | 0.0066 | 0.0254 | 98.34 |
| | XD-65732 | 0.0030 | 0.0149 | 0.0800 | 97.71 |
| | XD-65733 | 0.0020 | 0.0082 | 0.0359 | 97.48 |
| | XD-65734 | 0.0012 | 0.0053 | 0.0255 | 98.04 |
| | XD-65735 | 0.0015 | 0.0059 | 0.0243 | 98.36 |
| | XD-65736 | 0.0021 | 0.0120 | 0.0755 | 97.73 |
| | XD-65737 | 0.0020 | 0.0078 | 0.0328 | 98.01 |
| | XD-65738 | 0.0012 | 0.0052 | 0.0227 | 98.45 |
| | XD-65739 | 0.0019 | 0.0073 | 0.0295 | 98.34 |
| | XD-65740 | 0.0022 | 0.0103 | 0.0491 | 98.37 |
| 1051 | XD-65741 | 0.0006 | 0.0024 | 0.0099 | 98.75 |
| | XD-65742 | 0.0007 | 0.0025 | 0.0094 | 99.16 |
| | XD-65743 | 0.0004 | 0.0016 | 0.0057 | 98.97 |
| | XD-65744 | 0.0009 | 0.0034 | 0.0123 | 98.62 |
| | XD-65745 | 0.0007 | 0.0029 | 0.0128 | 98.91 |
| | XD-65746 | 0.0006 | 0.0023 | 0.0088 | 99.16 |
| | XD-65747 | 0.0005 | 0.0016 | 0.0055 | 98.96 |
| | XD-65748 | 0.0008 | 0.0031 | 0.0120 | 98.72 |
| | XD-65749 | 0.0008 | 0.0031 | 0.0123 | 98.76 |
| | XD-65750 | 0.0011 | 0.0041 | 0.0151 | 98.34 |
| | XD-65751 | 0.0009 | 0.0034 | 0.0131 | 97.57 |
| | XD-65752 | 0.0016 | 0.0060 | 0.0236 | 96.70 |
| | XD-65753 | 0.0008 | 0.0030 | 0.0118 | 98.41 |
| 1448 | XD-65754 | 0.0023 | 0.0089 | 0.0346 | 98.73 |
| | XD-65755 | 0.0014 | 0.0050 | 0.0190 | 99.09 |
| | XD-65756 | 0.0007 | 0.0026 | 0.0092 | 98.63 |
| | XD-65757 | 0.0007 | 0.0027 | 0.0105 | 99.13 |
| | XD-65758 | 0.0042 | 0.0158 | 0.0612 | 98.84 |
| | XD-65759 | 0.0015 | 0.0054 | 0.0200 | 98.87 |
| | XD-65760 | 0.0007 | 0.0025 | 0.0095 | 98.01 |
| | XD-65761 | 0.0008 | 0.0031 | 0.0120 | 98.99 |
| | XD-65762 | 0.0015 | 0.0060 | 0.0239 | 98.90 |
| | XD-65763 | 0.0009 | 0.0037 | 0.0152 | 98.86 |
| | XD-65764 | 0.0009 | 0.0036 | 0.0149 | 98.64 |
| | XD-65765 | 0.0011 | 0.0043 | 0.0174 | 98.70 |
| | XD-65766 | 0.0025 | 0.0104 | 0.0434 | 98.17 |
| 1627 | XD-65767 | 0.0024 | 0.0098 | 0.0413 | 98.60 |
| | XD-65768 | 0.0017 | 0.0063 | 0.0239 | 98.83 |
| | XD-65769 | 0.0022 | 0.0081 | 0.0308 | 98.33 |
| | XD-65770 | 0.0050 | 0.0179 | 0.0659 | 98.15 |
| | XD-65771 | 0.0026 | 0.0109 | 0.0475 | 98.46 |
| | XD-65772 | 0.0019 | 0.0069 | 0.0251 | 98.82 |
| | XD-65773 | 0.0023 | 0.0088 | 0.0349 | 98.45 |
| | XD-65774 | 0.0059 | 0.0202 | 0.0720 | 97.89 |
| | XD-65775 | 0.0020 | 0.0086 | 0.0376 | 98.26 |
| | XD-65776 | 0.0018 | 0.0068 | 0.0268 | 97.85 |
| | XD-65777 | 0.0023 | 0.0099 | 0.0446 | 95.31 |
| | XD-65778 | 0.0033 | 0.0119 | 0.0454 | 97.64 |
| | XD-65779 | 0.0027 | 0.0100 | 0.0387 | 97.26 |
| 1840 | XD-65780 | 0.0024 | 0.0114 | 0.0582 | 97.43 |
| | XD-65781 | 0.0024 | 0.0102 | 0.0464 | 97.31 |
| | XD-65782 | 0.0037 | 0.0177 | 0.0973 | 95.59 |
| | XD-65783 | 0.0024 | 0.0109 | 0.0528 | 97.17 |
| | XD-65784 | 0.0032 | 0.0138 | 0.0642 | 97.16 |
| | XD-65785 | 0.0020 | 0.0094 | 0.0468 | 97.04 |
| | XD-65786 | 0.0032 | 0.0175 | 0.1076 | 95.58 |
| | XD-65787 | 0.0026 | 0.0114 | 0.0530 | 97.17 |
| | XD-65788 | 0.0019 | 0.0097 | 0.0539 | 96.83 |
| | XD-65789 | 0.0048 | 0.0199 | 0.0927 | 95.76 |
| | XD-65790 | 0.0072 | 0.0368 | 0.3496 | 78.71 |
| | XD-65791 | 0.0034 | 0.0133 | 0.0561 | 97.17 |
| | XD-65792 | 0.0036 | 0.0148 | 0.0748 | 91.52 |
| 2157 | XD-65793 | 0.0086 | 0.0293 | 0.1043 | 97.86 |
| | XD-65794 | 0.0045 | 0.0144 | 0.0472 | 98.52 |
| | XD-65795 | 0.0022 | 0.0078 | 0.0284 | 98.61 |
| | XD-65796 | 0.0018 | 0.0059 | 0.0203 | 98.81 |
| | XD-65797 | 0.0068 | 0.0254 | 0.0990 | 97.63 |
| | XD-65798 | 0.0038 | 0.0123 | 0.0413 | 98.61 |
| | XD-65799 | 0.0018 | 0.0068 | 0.0267 | 98.61 |
| | XD-65800 | 0.0017 | 0.0058 | 0.0204 | 98.57 |
| | XD-65801 | 0.0025 | 0.0104 | 0.0453 | 98.55 |
| | XD-65802 | 0.0023 | 0.0086 | 0.0335 | 97.85 |
| | XD-65803 | 0.0023 | 0.0095 | 0.0410 | 98.26 |
| | XD-65804 | 0.0018 | 0.0062 | 0.0222 | 98.83 |
| | XD-65805 | 0.0035 | 0.0127 | 0.0479 | 97.27 |
| 2657 | XD-65806 | 0.0016 | 0.0074 | 0.0356 | 98.28 |
| | XD-65807 | 0.0016 | 0.0053 | 0.0178 | 98.34 |
| | XD-65808 | 0.0009 | 0.0037 | 0.0158 | 97.69 |
| | XD-65809 | 0.0015 | 0.0055 | 0.0203 | 98.86 |
| | XD-65810 | 0.0023 | 0.0096 | 0.0408 | 98.52 |
| | XD-65811 | 0.0019 | 0.0062 | 0.0209 | 98.48 |
| | XD-65812 | 0.0014 | 0.0050 | 0.0189 | 97.87 |
| | XD-65813 | 0.0019 | 0.0064 | 0.0217 | 98.76 |
| | XD-65814 | 0.0023 | 0.0097 | 0.0418 | 98.30 |
| | XD-65815 | 0.0027 | 0.0084 | 0.0270 | 98.39 |
| | XD-65816 | 0.0018 | 0.0068 | 0.0277 | 97.65 |
| | XD-65817 | 0.0028 | 0.0089 | 0.0288 | 98.34 |
| | XD-65818 | 0.0036 | 0.0134 | 0.0522 | 98.23 |

TABLE 6B
Summary of seed mediated off target TFR2 siRNA activity

| | | Seed x3 target sequence | | | |
|---|---|---|---|---|---|
| | | | | | Max- |
| Starting | | | | | imum |
| position | siRNA ID | IC20 (nM) | IC50 (nM) | IC80 (nM) | KD (%) |
| 241 | XD-65715 | 0.0651 | #N/A | #N/A | 42.47 |
| | XD-65716 | #N/A | #N/A | #N/A | −7.09 |
| | XD-65717 | #N/A | #N/A | #N/A | −4.42 |
| | XD-65718 | #N/A | #N/A | #N/A | −2.62 |
| | XD-65719 | 0.0601 | 2.5914 | #N/A | 53.58 |
| | XD-65720 | #N/A | #N/A | #N/A | −6.69 |
| | XD-65721 | #N/A | #N/A | #N/A | −7.99 |
| | XD-65722 | #N/A | #N/A | #N/A | −4.95 |
| | XD-65723 | 0.2547 | #N/A | #N/A | 47.37 |
| | XD-65724 | #N/A | #N/A | #N/A | −9.74 |
| | XD-65725 | #N/A | #N/A | #N/A | −5.08 |
| | XD-65726 | #N/A | #N/A | #N/A | −6.17 |
| | XD-65727 | 0.1013 | #N/A | #N/A | 40.86 |
| 412 | XD-65728 | 0.2934 | #N/A | #N/A | 41.35 |
| | XD-65729 | #N/A | #N/A | #N/A | 9.04 |
| | XD-65730 | #N/A | #N/A | #N/A | 12.99 |
| | XD-65731 | #N/A | #N/A | #N/A | 0.49 |
| | XD-65732 | 0.2834 | 1.7481 | #N/A | 54.64 |
| | XD-65733 | #N/A | #N/A | #N/A | 6.48 |
| | XD-65734 | #N/A | #N/A | #N/A | 12.68 |
| | XD-65735 | #N/A | #N/A | #N/A | 4.18 |
| | XD-65736 | 0.6580 | #N/A | #N/A | 40.86 |
| | XD-65737 | #N/A | #N/A | #N/A | 9.44 |
| | XD-65738 | #N/A | #N/A | #N/A | 6.59 |
| | XD-65739 | #N/A | #N/A | #N/A | 6.52 |
| | XD-65740 | 0.6915 | #N/A | #N/A | 42.75 |
| 1051 | XD-65741 | 0.1267 | 0.4441 | #N/A | 63.65 |
| | XD-65742 | 0.3552 | #N/A | #N/A | 25.47 |
| | XD-65743 | #N/A | #N/A | #N/A | 2.14 |
| | XD-65744 | 0.3650 | #N/A | #N/A | 25.39 |
| | XD-65745 | 0.0851 | 0.2926 | #N/A | 73.75 |
| | XD-65746 | 0.3404 | #N/A | #N/A | 24.73 |
| | XD-65747 | #N/A | #N/A | #N/A | 6.80 |
| | XD-65748 | 0.3163 | #N/A | #N/A | 31.80 |
| | XD-65749 | 0.1007 | 0.3076 | #N/A | 72.99 |
| | XD-65750 | 0.4116 | #N/A | #N/A | 27.19 |
| | XD-65751 | 2.8934 | #N/A | #N/A | 21.77 |
| | XD-65752 | 0.3391 | #N/A | #N/A | 33.07 |
| | XD-65753 | 0.0766 | 0.2142 | #N/A | 73.00 |
| 1448 | XD-65754 | 0.1238 | 0.3490 | 6.6285 | 76.02 |
| | XD-65755 | 0.4644 | 9.4304 | #N/A | 51.21 |
| | XD-65756 | #N/A | #N/A | #N/A | 6.99 |
| | XD-65757 | #N/A | #N/A | #N/A | 19.34 |
| | XD-65758 | 0.2332 | 0.4961 | #N/A | 78.63 |
| | XD-65759 | 1.1316 | #N/A | #N/A | 42.24 |
| | XD-65760 | #N/A | #N/A | #N/A | 6.75 |
| | XD-65761 | #N/A | #N/A | #N/A | 19.93 |
| | XD-65762 | 0.1003 | 0.3274 | 11.2642 | 80.38 |
| | XD-65763 | #N/A | #N/A | #N/A | 12.81 |
| | XD-65764 | #N/A | #N/A | #N/A | 6.28 |
| | XD-65765 | 34477.4796 | #N/A | #N/A | 20.30 |
| | XD-65766 | 0.1849 | 0.3060 | #N/A | 77.93 |
| 1627 | XD-65767 | 0.0642 | 0.2329 | 2.2049 | 81.58 |
| | XD-65768 | 0.4911 | #N/A | #N/A | 38.69 |
| | XD-65769 | #N/A | #N/A | #N/A | 9.51 |
| | XD-65770 | #N/A | #N/A | #N/A | 6.02 |
| | XD-65771 | 0.0650 | 0.1968 | 1.2013 | 80.82 |
| | XD-65772 | 0.5471 | #N/A | #N/A | 29.88 |
| | XD-65773 | #N/A | #N/A | #N/A | 9.07 |
| | XD-65774 | #N/A | #N/A | #N/A | 0.81 |
| | XD-65775 | 0.0456 | 0.1313 | 0.7961 | 82.09 |
| | XD-65776 | 0.5101 | #N/A | #N/A | 44.75 |
| | XD-65777 | #N/A | #N/A | #N/A | 5.57 |
| | XD-65778 | #N/A | #N/A | #N/A | 4.53 |
| | XD-65779 | 0.0394 | 0.1193 | 0.7078 | 83.02 |
| 1840 | XD-65780 | 0.3495 | #N/A | #N/A | 32.03 |
| | XD-65781 | #N/A | #N/A | #N/A | −6.46 |
| | XD-65782 | #N/A | #N/A | #N/A | −24.47 |
| | XD-65783 | #N/A | #N/A | #N/A | −7.69 |
| | XD-65784 | 0.3551 | #N/A | #N/A | 36.07 |
| | XD-65785 | #N/A | #N/A | #N/A | −10.03 |
| | XD-65786 | #N/A | #N/A | #N/A | −22.32 |
| | XD-65787 | #N/A | #N/A | #N/A | −1.87 |
| | XD-65788 | 0.4652 | #N/A | #N/A | 34.36 |
| | XD-65789 | #N/A | #N/A | #N/A | −8.75 |
| | XD-65790 | #N/A | #N/A | #N/A | −17.97 |
| | XD-65791 | #N/A | #N/A | #N/A | 3.38 |
| | XD-65792 | 1.2680 | #N/A | #N/A | 32.12 |
| 2157 | XD-65793 | 0.0187 | 0.0562 | 0.1942 | 92.95 |
| | XD-65794 | 0.0247 | 0.0672 | 0.2123 | 92.11 |
| | XD-65795 | 0.0349 | 0.1054 | 0.3882 | 91.17 |
| | XD-65796 | 0.0333 | 0.0895 | 0.2903 | 91.43 |
| | XD-65797 | 0.0188 | 0.0571 | 0.2043 | 92.68 |
| | XD-65798 | 0.0405 | 0.1100 | 0.3564 | 90.86 |
| | XD-65799 | 0.0344 | 0.1324 | 0.6888 | 89.53 |
| | XD-65800 | 0.0378 | 0.1204 | 0.5060 | 89.42 |
| | XD-65801 | 0.0245 | 0.0588 | 0.1632 | 92.59 |
| | XD-65802 | 0.1045 | 0.2451 | 0.7444 | 88.97 |
| | XD-65803 | 0.2530 | 0.6566 | #N/A | 72.20 |
| | XD-65804 | 0.0790 | 0.2153 | 0.7437 | 90.55 |
| | XD-65805 | 0.0191 | 0.0465 | 0.1334 | 91.56 |
| 2657 | XD-65806 | 0.1160 | 0.3644 | #N/A | 75.60 |
| | XD-65807 | #N/A | #N/A | #N/A | 6.52 |
| | XD-65808 | #N/A | #N/A | #N/A | −6.18 |
| | XD-65809 | #N/A | #N/A | #N/A | 10.11 |
| | XD-65810 | 0.0438 | 0.1365 | 0.9680 | 82.98 |
| | XD-65811 | #N/A | #N/A | #N/A | 11.22 |
| | XD-65812 | #N/A | #N/A | #N/A | 4.92 |
| | XD-65813 | #N/A | #N/A | #N/A | 13.26 |
| | XD-65814 | 0.1140 | 0.3129 | #N/A | 77.50 |
| | XD-65815 | #N/A | #N/A | #N/A | 6.16 |
| | XD-65816 | #N/A | #N/A | #N/A | 0.65 |
| | XD-65817 | #N/A | #N/A | #N/A | 10.08 |
| | XD-65818 | 0.1130 | 0.2445 | #N/A | 79.29 |

### Example 5: Analysis of Activity of Chemically Optimized TFR2 siRNAs in Primary Human Hepatocytes (PHH) by Free Uptake Mediated by a GalNAc Ligand

Based on the results of the dual-luciferase assay for on-/off-target activity, best performing siRNAs that displayed high on-target activity and little to no seed-mediated activity were tested for the RNAi activity towards TFR2 mRNA in PHH using a GalNAc-mediated free uptake delivery (Tables 7 and 8).

Plated primary human hepatocytes pooled from 5 donors (XenoTech, HPCH05+) were exposed to 7 different concentrations of siRNAs from 1 pM to 0.064 nM in 5-fold dilution steps in triplicates. Non-treated cells were used as negative control. After 72 hours of exposure, the cells were washed, lysed and RNA isolated. Harvested RNA was reverse transcribed and assayed for TFR2 expression via TaqMan qPCR. A single qPCR assay was performed for each sample using a TFR2 TaqMan probe set (Hs00162690_m1-FAM) multiplexed with a common GAPDH-VIC probe (ThermoFisher, Hs99999905_m1-VIC). Thermocycling and data acquisition was performed with an Applied Biosystems QuantStudio 3 Real-Time PCR System and analyzed. Cq was measured at the auto-defined threshold for each TFR2-FAM and GAPDH-VIC amplification curve. A 5-fold dilution series (1:1, 1:5, 1:25) of a common stock of non-treated cell RNA was assayed in each qPCR plate. A standard curve was calculated by plotting the Cq derived for each dilution against the base 2 log of the dilution factor, and a linear trendline calculated for each probe. Each Cq data point “y” was then normalized to the corresponding trendline by solving for “x”. The base 2 antilog of each data point was then taken, and the TFR2-FAM values divided by the corresponding GAPDH-VIC values for each well and averaged as the mean of three replicates to obtain average Rq values per sample. These values were all normalized to the mean Rq of control non-treated cells to obtain the mean relative abundance as a percentage of expression in non-treated cells (the results are shown in Table 7).

Derived mean and standard error data was copied to GraphPad Prism statistical analysis software (version 9.5.1). IC50 values for the dose curves were calculated via nonlinear regression using the “[Inhibitor] vs. normalized response—Variable slope” calculation set. This equation assumes data that can be normalized to a 0-100% scale and calculates a non-standard Hill slope to the curve. The results of this analysis are shown in Table 7.

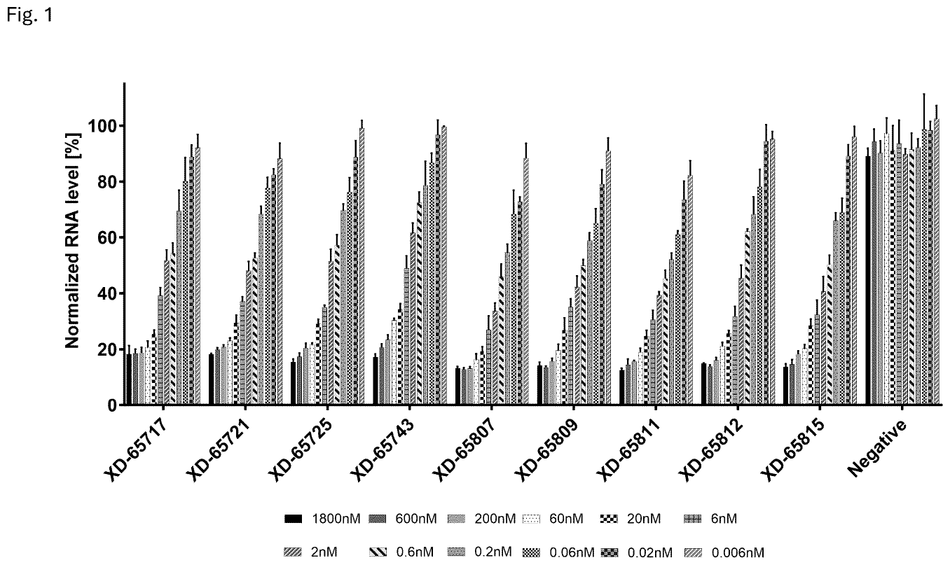

To better define the IC50 values, a repeated siRNA free uptake experiment in PHH was conducted with expanded 14-point siRNA dose range from 1. μM to 6 pM in 3-fold dilution steps with the best performing siRNAs. The results are summarized in Table 8 and FIG. 1.

TABLE 7
IC50 concentrations and maximum TFR2 knockdown
activity of TFR2 siRNAs in PHH (7-point dose curves)

| Starting | | | |
|---|---|---|---|
| position | siIRNA ID | IC50 (nM) | Maximum KD (%) |
| 241 | XD-65716 | 17.38 | 69.24 |
| | XD-65717 | 2.132 | 74.51 |
| | XD-65718 | 4.179 | 77.50 |
| | XD-65719 | 2.536 | 81.37 |
| | XD-65720 | 23.11 | 72.87 |
| | XD-65721 | 2.555 | 81.91 |
| | XD-65722 | 5.744 | 79.44 |
| | XD-65723 | 1.283 | 78.48 |
| | XD-65724 | 71.42 | 65.05 |
| | XD-65725 | 2.434 | 79.70 |
| | XD-65726 | 13.81 | 79.04 |
| 412 | XD-65728 | 1400 | 43.04 |
| | XD-65729 | 66.89 | 68.74 |
| | XD-65730 | 174.5 | 63.07 |
| | XD-65731 | 50.66 | 72.25 |
| | XD-65732 | 699.3 | 51.61 |
| | XD-65733 | 68.9 | 67.45 |
| | XD-65734 | 73.6 | 69.95 |
| | XD-65735 | 73.83 | 69.42 |
| | XD-65736 | 32.02 | 71.18 |
| | XD-65737 | 17.66 | 73.34 |
| | XD-65738 | 45.39 | 69.67 |
| | XD-65739 | 13.82 | 79.86 |
| 1051 | XD-65741 | 4.941 | 79.37 |
| | XD-65743 | 4.321 | 79.90 |
| | XD-65745 | 15.9 | 79.30 |
| | XD-65747 | 6.361 | 80.75 |
| 1448 | XD-65754 | 21.2 | 64.93 |
| | XD-65756 | 41.33 | 69.90 |
| | XD-65758 | 417.5 | 55.64 |
| | XD-65760 | 103.4 | 68.43 |
| | XD-65762 | 99.84 | 65.66 |
| | XD-65763 | 203.7 | 62.88 |
| | XD-65764 | 376.2 | 54.44 |
| 1627 | XD-65767 | 175.3 | 64.35 |
| | XD-65769 | 46.11 | 72.37 |
| | XD-65770 | 30.43 | 71.51 |
| | XD-65771 | 14.13 | 69.19 |
| | XD-65773 | 77.12 | 67.89 |
| | XD-65774 | 305.3 | 58.74 |
| | XD-65775 | 96.89 | 63.05 |
| | XD-65777 | 319.7 | 60.54 |
| | XD-65778 | 221.1 | 59.63 |
| 1840 | XD-65780 | 11.65 | 77.60 |
| | XD-65781 | 42.65 | 64.70 |
| | XD-65782 | 11.19 | 74.50 |
| | XD-65783 | 7.092 | 82.20 |
| | XD-65784 | 19.02 | 72.27 |
| | XD-65785 | 5.842 | 76.30 |
| | XD-65786 | 39.43 | 68.07 |
| | XD-65787 | 80.46 | 68.32 |
| | XD-65788 | 141.1 | 61.89 |
| | XD-65789 | 394.5 | 55.69 |
| | XD-65790 | 407.3 | 57.46 |
| | XD-65791 | 50.64 | 73.69 |
| 2657 | XD-65806 | 3.001 | 76.78 |
| | XD-65807 | 2.159 | 78.73 |
| | XD-65808 | 3.885 | 76.19 |
| | XD-65809 | 1.496 | 78.75 |
| | XD-65810 | 1.695 | 78.66 |
| | XD-65811 | 0.8179 | 80.65 |
| | XD-65812 | 2.695 | 78.80 |
| | XD-65813 | 2.083 | 76.41 |
| | XD-65814 | 1.494 | 77.50 |
| | XD-65815 | 2.708 | 78.36 |
| | XD-65816 | 8.771 | 74.56 |
| | XD-65817 | 14.52 | 73.86 |

TABLE 8
IC50 concentrations and maximum TFR2 knockdown activity (100%-%
residual mRNA) of TFR2 siRNAs in PHH (expanded 14-point dose curve)

| Starting | | IC50 | |
|---|---|---|---|
| position | siRNA ID | (nM) | Maximum KD (%) |
| 241 | XD-65717 | 2.08 | 81.67 |
| | XD-65721 | 1.81 | 81.79 |
| | XD-65725 | 2.15 | 84.45 |
| 1051 | XD-65743 | 7.27 | 82.77 |
| 2657 | XD-65807 | 0.44 | 86.73 |
| | XD-65809 | 0.83 | 85.78 |
| | XD-65811 | 0.43 | 87.41 |
| | XD-65812 | 1.77 | 84.99 |
| | XD-65815 | 1.18 | 86.24 |

Three siRNAs starting from position 2657 of TFR2 mRNA, all having 2′-5′ linkage between positions 7 and 8 of the antisense strand and 3′H in position 7 but differing in the number of 2′Ome modifications in their antisense strands (XD65815>XD65811>XD65807) were selected for in vivo analysis keeping in mind that having more 2′Ome modifications may improve duration of effect. Notably, the results from the on-target screening using the psiCHECK-based assay (Table 6A) did not point that specifically these siRNAs will show the best KD performance in PHH.

### Example 6: Assessment of TFR2 siRNA Pharmacodynamic Activity In Vivo in Mice with Humanized Liver Tissue

This example presents a study that evaluated the knockdown effects in vivo in the livers of three GalNAc-conjugated siRNA compounds, XD-65807, XD-65811 and XD-65815, targeting human TFR2 mRNA. The study was conducted in uPA-SCID mice (PXB) mice in which mouse hepatocytes have been substituted with human hepatocytes (PhoenixBio Co, Japan). Each mouse accepted in the study had at least 90% of hepatocytes replaced as confirmed by the vendor.

Each mouse received a single subcutaneous injection of either a vehicle (PBS) or of one of the three siRNAs—each at 3 dose levels of 5, 10 or 30 mg/kg to be analyzed for gene expression in the liver at 15 days post dose. Additional groups of mice were treated with 10 mg/kg doses of each of the siRNAs or the vehicle to be analyzed for gene expression in the liver at 49 days post dose. Each study group consisted of 4-5 mice. At the designated time points, the livers were harvested for RNA extraction from each lobe separately and gene expression analysis using RT-qPCR. RT-qPCR was performed in technical triplicates using Taqman Hs00162690_m1-FAM probe for human TFR2 combined with TaqMan Endogenous Control; VIC®/MGB Hs99999905_m1 Probe for GAPDH (all from ThermoFisher), essentially as described in Example 5. Equal quantities of each PBS control sample from each of the three liver lobes were combined into a pool for control dilutions and expression normalization on all qPCR plates.

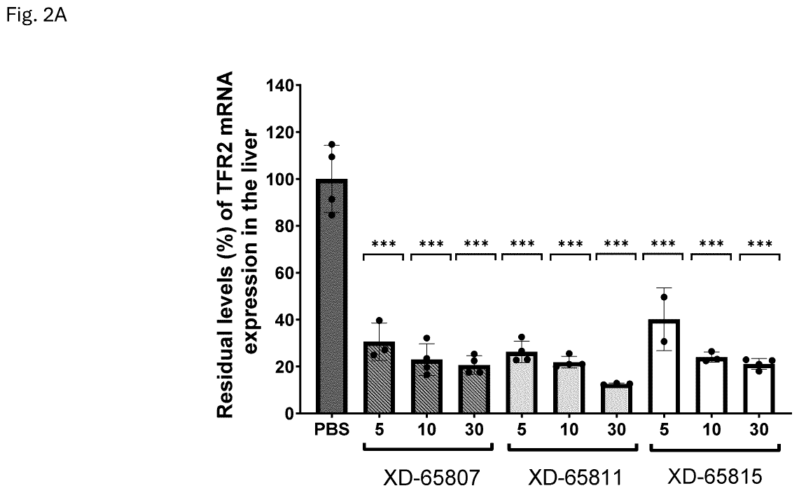

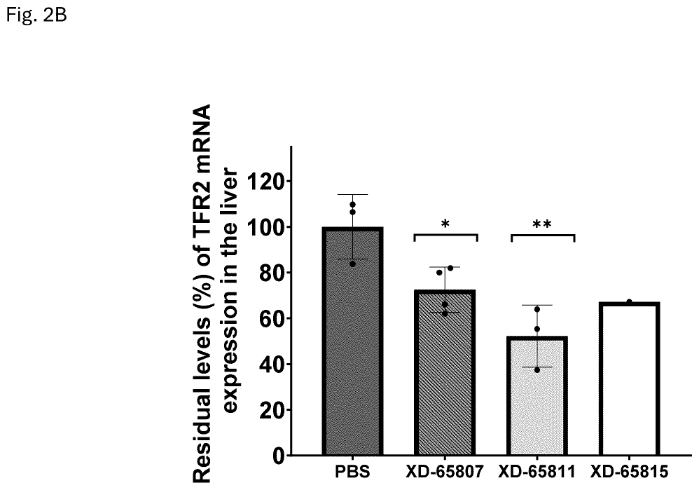

Overall, the siRNAs at all dose levels were well-tolerated. The results of TFR2 gene expression in the liver on Day 15 are shown in FIG. 2A and on Day 49—in FIG. 2B.

As shown in FIG. 2A, on Day 15, treatment with all the three siRNAs at all three dose levels have statistically significantly (p<0.0001) reduced human TFR2 mRNA expression in the liver in a dose-dependent manner. Even the lowest 5 mg/kg doses reduced human TFR2 mRNA expression by 60-70%. The most effective treatment was with siRNA XD-65811, achieving an 86% reduction at 30 mg/kg. siRNA XD65815 was the least effective. By Day 49 (FIG. 2B), for 10 mg/kg dose levels, the knockdown (KD) rebound was observed. The average residual mRNA levels increased from ˜25% on Day 15 to ˜70-75% on Day 49 for siRNAs XD-65807 and XD-65815, and from ˜25% on Day 15 to ˜54% on Day 49 for siRNA XD-65811. The TFR2 KD effects of siRNAs XD-65807 and XD-65811 on Day 49 were still statistically significant (p<0.05 and p<0.01, respectively). Statistical significance of the TFR2 knockdown by siRNA XD-65815 was unevaluable because only a single animal (n=1) remained in this group by the time of the analysis (note that mortality in study groups was not study drug- or dose-dependent and likely stemmed from external factors, i.e., transportation and housing conditions, known to have a high impact on the well-being of the fragile PXB mice). There was minimal intra- and inter-animal variability in TFR2 mRNA expression within all dose groups at all analysis timepoints.

### Example 7: Evaluation of Activity of TFR2 siRNAs in Cynomolgus Monkeys Exposed to the Model of Acute Inflammation Induced by IL-6 Treatment

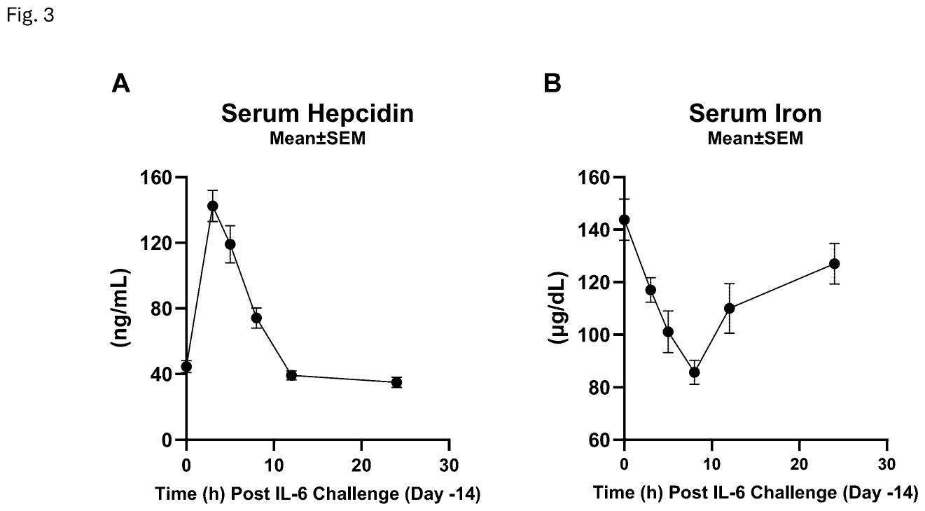

Twelve healthy male cynomolgus monkeys were assigned to four treatment groups (n=3). On day −14, all study animals were challenged with a single dose of IL-6 (1 ag/kg, intravenously) and frequently sampled for serum hepcidin and iron levels over the period of 24 hours. IL-6 treatment induced acute time-dependent increase in serum hepcidin levels and decrease in serum iron levels, peaking at 3 and 8 hours post dose, respectively (FIG. 3).

On Day 0, all animals received their designated treatment with single subcutaneous doses of either XD-65807 or XD-65811 TFR2 siRNAs at either 1 mg/kg or 5 mg/kg via subcutaneous injection. On Day 28, all animals received a second intravenous dose of IL-6 with frequent sampling for serum hepcidin and iron levels over the period of 24 hours thereafter.

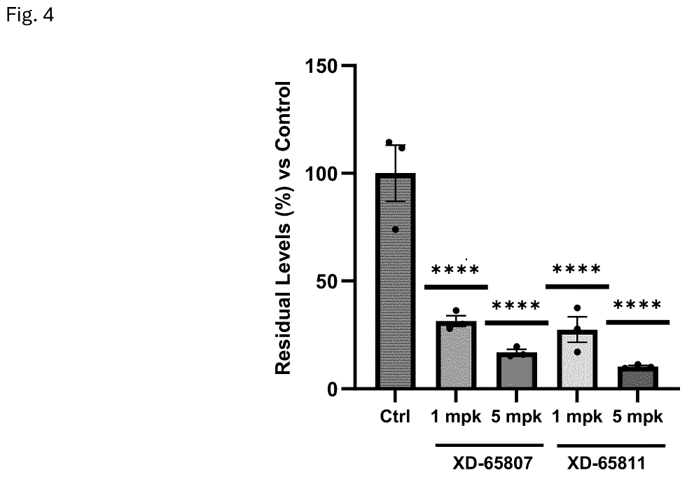

The siRNA-mediated TFR2 knockdown in the livers of treated animals was confirmed by comparing TFR2 mRNA expression levels in liver biopsies of the treated monkeys performed 6 weeks post dose vs liver biopsies from a separate cohort (n=3) of similar but non-treated animals that did not otherwise participate in the study (FIG. 4). Treatment with XD-65807 and XD-65811 was well-tolerated and resulted in a dose-dependent statistically significant hepatic TFR2 mRNA knockdown compared to untreated controls—up to 72.5% and 90% (XD-65811, p<0.0001) with 1 mg/kg and 5 mg/kg doses, respectively.

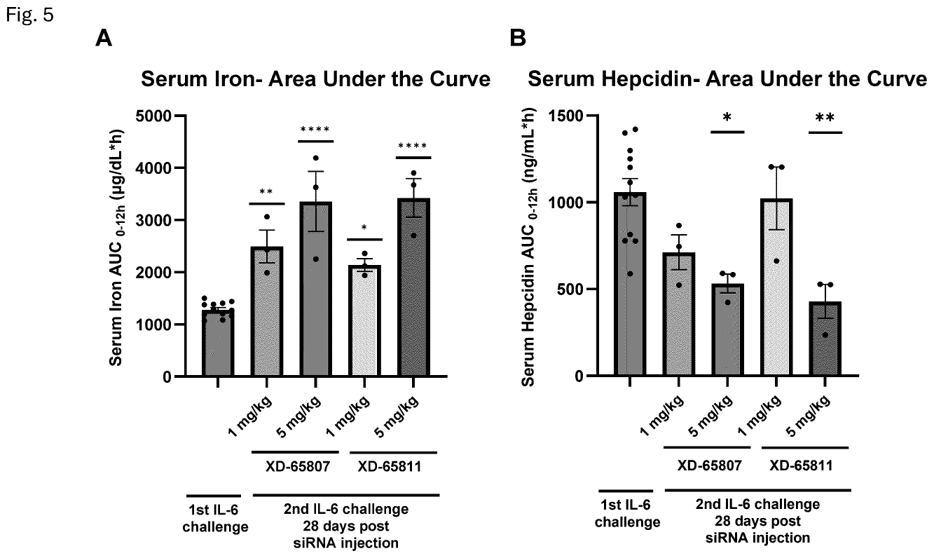

To assess the effect of siRNA-mediated TFR2 knockdown in the liver on IL-6-induced hypoferremia and hepcidin elevation, the areas under the concentration-time curve over the 12-hour observation period (AUC₀₋₁₂) for IL-6-elicited changes in serum hepcidin (panel B) and iron levels (panel A) on Day −14 (the 1^{st} IL-6 treatment) were compared to those on Day 28 (the 2^{nd} IL-6 treatment) (FIG. 5). As evident, the elicited increase in serum hepcidin levels was significantly reduced at 5 mg/kg dose of either siRNA, whereas the decrease of serum iron levels was significantly attenuated in a dose-dependent manner.

### Example 8: Evaluation of TFR2 siRNAs Activity In Vivo in Cynomolgus Macaques Over Time after Exposure to the Model of Acute Inflammation

Monkeys described in Example 7 were followed up to week 22 post siRNA administration for liver TFR2 knockdown, serum hepcidin, iron levels (absolute and transferrin saturation), and red blood parameters.

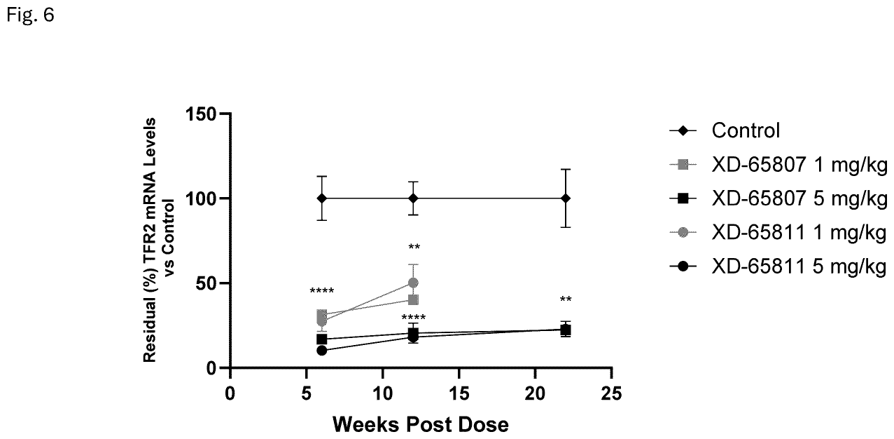

For TFR2 knockdown (KD) assessment, liver biopsies were collected at 6 and 12 weeks (all animals) and at 22 weeks (5 mg/kg groups only). Control liver biopsy samples were obtained from a separate cohort of similar but untreated animals (n=3) (FIG. 6).

Even at 22 weeks post-dose, TFR2 mRNA levels in the high dose groups (5 mg/kg) remained deeply suppressed for both siRNAs tested. (both siRNAs showed a maximum 77% knockdown; p<0.01).

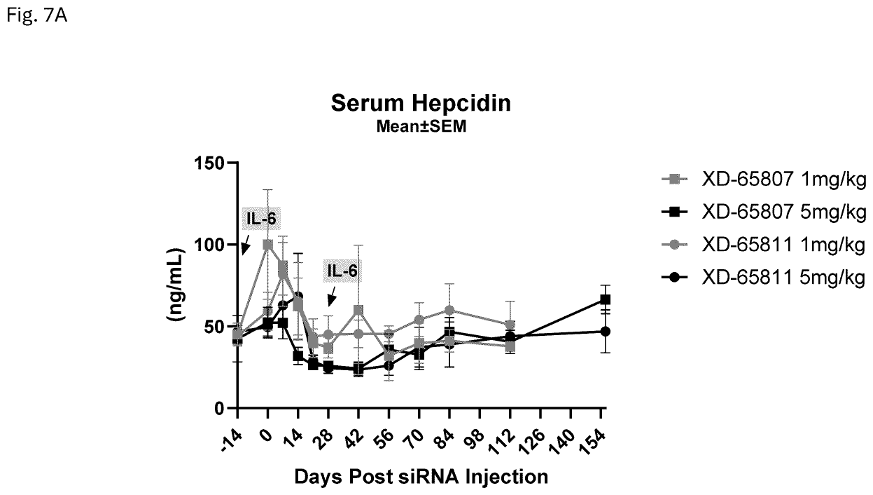

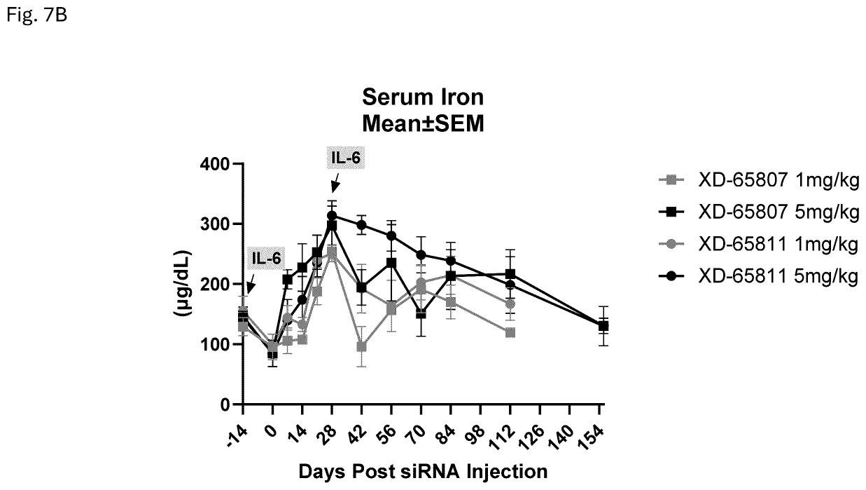

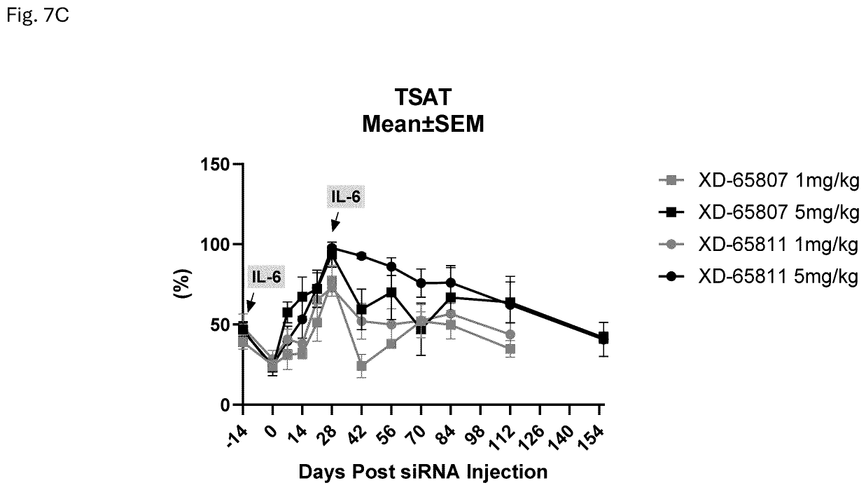

siRNA-mediated TFR2 mRNA knockdown in monkey livers was associated with dose-dependent decreases in serum hepcidin (FIG. 7A) starting from 3-4 weeks after siRNA administration. Decreased serum hepcidin was sustained at nadir levels until week 8 with a slow return to baseline thereafter.

Notably, an initial increase in serum hepcidin observed following the IL-6 challenge on Day −14, was associated, as expected, with a decrease in serum iron (FIG. 7B) and transferrin saturation (TSAT) (FIG. 7C). Injection of either XD-65807 or XD-65811 on Day 0 resulted in a subsequent gradual increase in serum iron and TSAT over the first 28 days post siRNA treatment indicating successful iron mobilization. The peak response of 2-3-fold increase in serum iron, and TSAT was observed at 4 weeks post siRNA dosing (prior to the second IL-6 challenge). Importantly, the second IL-6 challenge did not result in the same impact on serum hepcidin and iron levels, suggesting that treatment with TFR2-siRNA led to attenuation of hepcidin/iron responses to acute inflammation (FIGS. 5 and 7A-7C). Remarkably, by week 16 (Day 112), serum iron and TSAT levels had not yet returned to baseline even in the low-dose (1 mg/kg) groups.

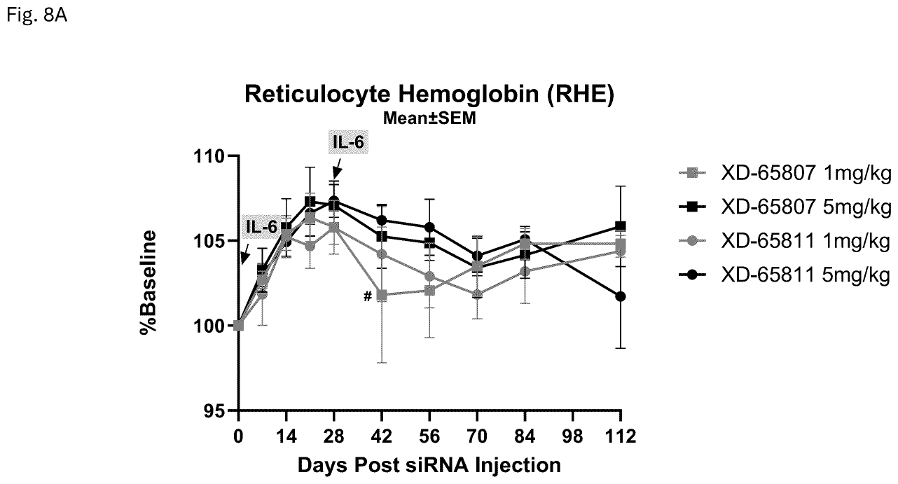

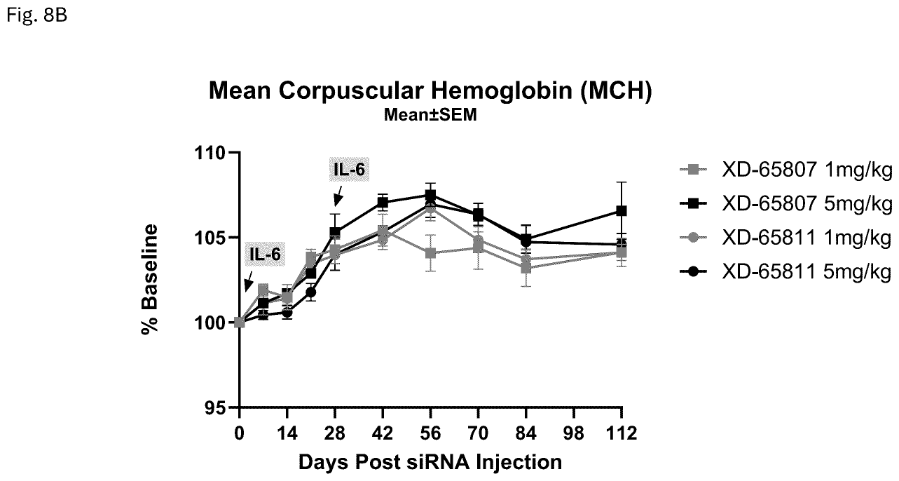

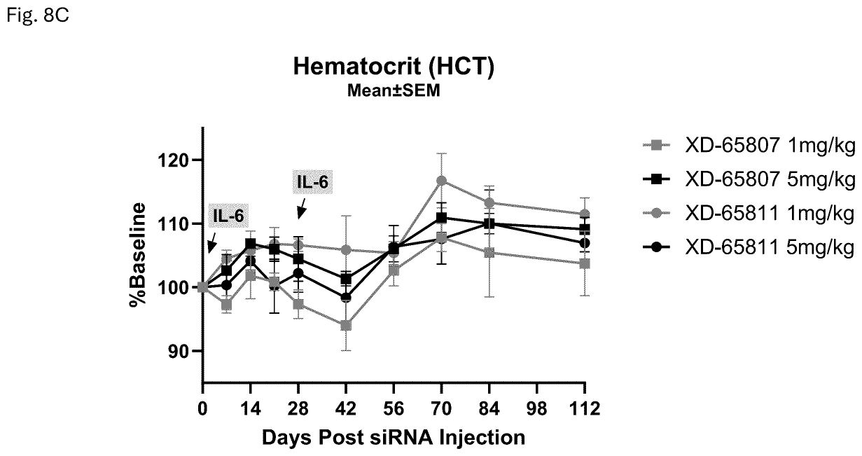

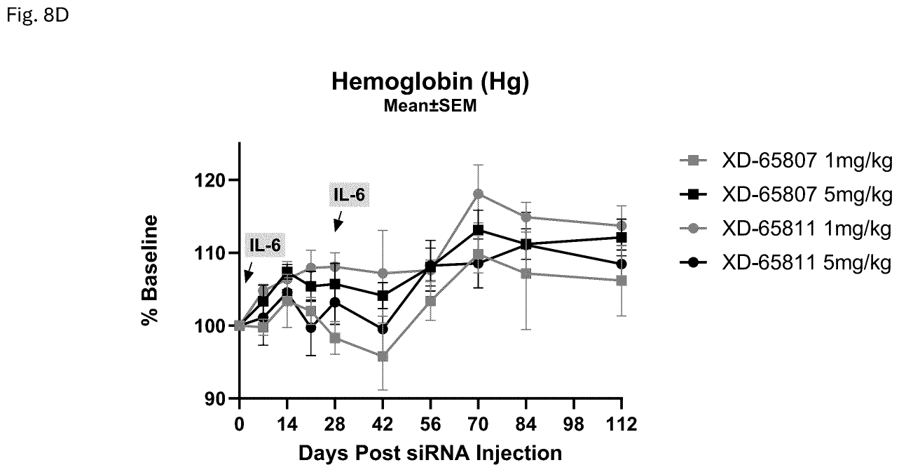

The observed increase in serum iron availability was translated into accelerated erythropoiesis, evident from early increases of markers of erythroid lineage proliferation and erythrocyte hemoglobinization—the reticulocyte hemoglobin (RHE) (FIG. 8A) and mean corpuscular hemoglobin (MCH) levels (FIG. 6B). This was followed, at later time points, by subsequent increases in hematocrit (HCT) (FIG. 6C) and total hemoglobin (Hb) (FIG. 8D), peaking at 70 to 84 days following single administrations of XD-65807 or XD-65811.

### Example 9: Evaluation of Activity of Repeated Administrations of TFR2 siRNA in Normal Cynomolgus Monkeys

Examples 7 and 8 evaluated the in vivo effect of administrating TFR2 siRNA in the context of a model of acute inflammation. In this example, the effect of TFR2 siRNA in normal (i.e., non-stimulated) monkeys was assessed.

The study included sixteen healthy male cynomolgus monkeys, allocated into six treatment groups (n=2-3). On Day 0 and Week 12, Group 1 received subcutaneous injections of phosphate-buffered saline (PBS), while Groups 2 to 6 were administered with subcutaneous injections of TFR2 siRNA XD-65811 at dose levels of 0.3, 1.0, 3.0, 50.0, or 300.0 mg/kg, respectively. All XD-65811 administrations were well-tolerated—based both on clinical observations and laboratory parameters.

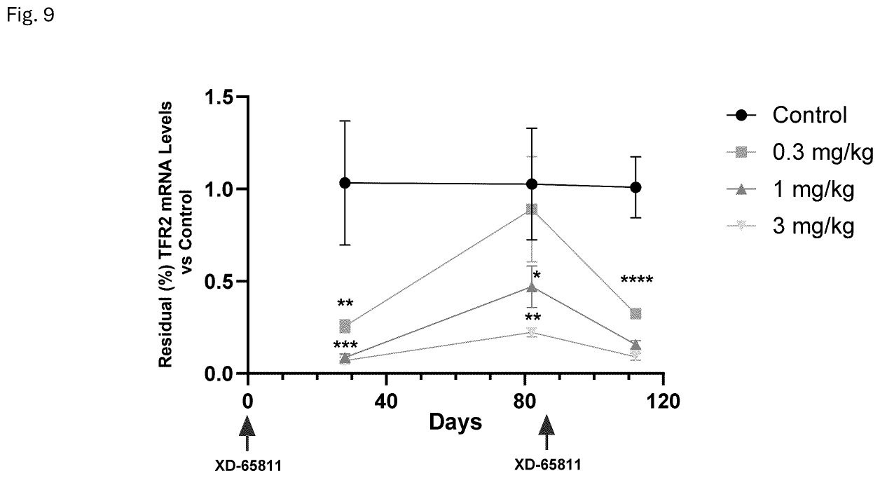

Liver biopsies were collected on Weeks 4, 12 and 16 post-dose from Groups 1-4 treated with 0-3 mg/kg XD-65811 for quantification of TFR2 mRNA levels using real-time RT-PCR. Treatment with XD-65811 resulted in a dose-dependent, statistically significant, TFR2 mRNA knockdown in all studied siRNA-treated groups compared to vehicle-treated controls that achieved maximum of 90% by Week 4. The minimum effective dose was 0.3 mg/kg. By Week 12 (prior to the second dose of XD-65811, a partial dose-dependent rebound of the knockdown activity was observed with the maintenance of 50% and 75% mRNA knockdown at dose levels of 1 and 3 mg/kg, respectively. By Week 16 (4 weeks after the second dose of TFR2 siRNA), the TFR2 mRNA knockdown at different dose levels was similar to that at 4 weeks after the first siRNA dose (FIG. 9).

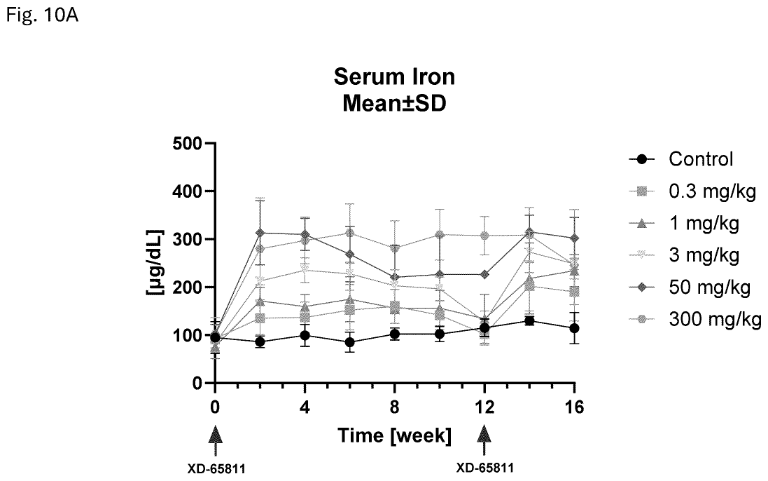

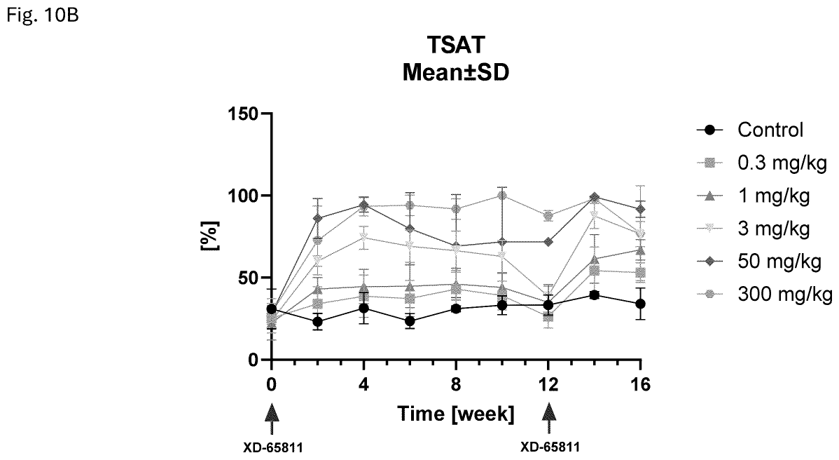

Administration of TFR2 siRNA led to time- and dose-dependent changes in serum iron (FIG. 10A) and TSAT (FIG. 10B). For both parameters, the minimum effective dose was 0.3 mg/kg of XD-65811, the plateau of the effect magnitude was achieved at doses>50 mg/kg whereas the highest dose of 300 mg/kg supported the longest duration of iron mobilizing effect.

After the first TFR2 siRNA dose, serum iron levels peaked in a dose-dependent manner by Week 2 after siRNA administration. The plateau of the effect magnitude was achieved at doses 50 mg/kg. Serum iron remained at the plateau levels till Week 8 for the 0.3 and 1 mg/kg dose groups, Week 6 for the 3 mg/kg dose group, and Week 4 for the 50 mg/kg dose groups, when a slow decline was initiated. However, at all dose levels, serum iron remained above baseline in a dose-dependent manner at least until Week 10. By the time of the second XD-65811 dose (Week 12), serum iron levels in all dose groups but 300 mg/kg, where it remained constantly high throughout the whole period, returned to baseline. The kinetics of serum iron elevations in response to the second administrations of XD-65811 was similar to that after the first dose but the magnitude of dose-dependent effects was higher for all dose levels 50 mg/kg with the plateau reached at 3 mg/kg of XD-65811 (FIG. 10A).

After the first TFR2 siRNA dose, TSAT levels peaked by Week 2 in two lower dose groups and by Week 4 in three higher dose groups. The plateau of the effect magnitude (close to 100%) was achieved at doses>50 mg/kg. For doses 0.3 and 1 mg/kg, TSAT remained at the respective plateau levels till the time of the second injection (Week 12), after which it was further increased in a dose-dependent manner by Week 16. For dose levels 3 and 50 mg/kg, there was a slight decrease of TSAT from the peak levels by Week 8 with the maintenance through Week 12 (still remaining at ˜2-fold above baseline). After the second siRNA dose, the response of TSAT to 50 mg/kg was maximal (close to 100%) whereas the response to 3 mg/kg was of a greater magnitude compared to the first dose. The TSAT levels after 300 mg/kg siRNA, once reaching the plateau after the first dose, were maintained throughout the study (FIG. 10B).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for treating a HAMP-related anemia, comprising:
administering to a subject in need thereof, an effective amount of a double-stranded nucleic acid comprising a sense strand and an antisense strand, wherein the sense strand comprises a sense strand sequence comprising:
5′-(C3)csgsguCfaUfAfCfugucgguuaa 3′ (SEQ ID NO: 308) and has three or four GalNAc moieties covalently attached to the 3′ end,
wherein the antisense strand comprises an antisense strand sequence comprising:
5′-(vinu)sUfsaacCf(3dG)AfCfaguaUfgAfccgsusc-3′ (SEQ ID NO: 293) or 5′-(vinu)sUfsaacCf(3dG)aCfaguaUfgAfccgsusc-3′ (SEQ ID NO: 297),
wherein a lowercase letter indicates a 2′-O-Methyl (2′-OMe) modified RNA nucleotide, an uppercase letter followed by f indicates a 2′-Fluoro (2′-F) modified RNA nucleotide, (C3) indicates a propanol or C3 alkyl moiety connected to the 5′ end of the strand via a phosphodiester linkage, s indicates a phosphorothioate linkage between adjacent nucleotides, (vinu) indicates a 5′ vinylphosphonate 2′-OMe RNA U nucleotide, and (3dG) indicates a G nucleotide in which ribose has H at the 3′ position, and is connected via a 2′-5′ bridge to a nucleotide following the G nucleotide,
thereby treating the anemia.

2. The method of claim 1, wherein the three or four GalNAc moieties are covalently attached to the 3′ end of the sense strand via a triethyleneglycol (TEG) linker.

3. The method of claim 1, wherein the sense strand sequence comprises:
5′-(C3)csgsguCfaUfAfCfugucgguuaasLsLsL-3 (SEQ ID NO: 291),
wherein a lowercase letter indicates a 2′-O-Methyl (2′-OMe) modified RNA nucleotide, an uppercase letter followed by f indicates a 2′-Fluoro (2′-F) modified RNA nucleotide, (C3) indicates a propanol or C3 alkyl moiety connected to the 5′ end of the strand via a phosphodiester linkage, s indicates a phosphorothioate linkage between adjacent nucleotides, and L indicates a monomer of N-acetylgalactosamine (GalNAc) with a TEG linker.

4. The method of claim 1, wherein the HAMP-related anemia is an iron-refractory anemia, anemia of a chronic disease, or an anemia of inflammation.

5. The method of claim 1, wherein the HAMP-related anemia is an anemia arising from cancer or cancer treatment.

6. The method of claim 5, wherein the cancer is a solid tumor or advanced-stage cancer.

7. The method of claim 5, wherein the cancer is a hematology-related cancer.

8. The method of claim 7, wherein the hematology-related cancer is transfusion dependent myelofibrosis, transfusion independent myelofibrosis, multiple myeloma, Castelman disease, Hodgkin lymphoma, non-Hodgkin lymphoma, leukemia, myelodysplastic syndrome, myeloproliferative neoplasms, or Waldenstrom macroglobulinemia.

9. The method of claim 4, wherein the anemia of inflammation is in a subject diagnosed with rheumatoid arthritis, inflammatory bowel disease, systemic lupus erythematosus, scleroderma psoriatic arthritis, vasculitides, autoimmune thyroiditis, anti-neutrophil cytoplasmic antibody associated vasculitis, kryoglobulinemia, chronic kidney disease, chronic inflammation, obesity, metabolic syndrome, a chronic infection, chronic obstructive pulmonary disease, and chronic graft versus host disease.

10. The method of claim 1, wherein the HAMP-related anemia arises from mutations in the TMPRSS6 gene.

11. The method of claim 1, wherein the subject has a condition that results in overproduction or reduced elimination of hepcidin.

12. The method of claim 1, wherein the subject is receiving or has previously received a treatment for anemia and is non- or poorly responsive to the treatment.

13. The method of claim 12, wherein the double-stranded nucleic acid is administered to the subject, before, concurrently with, or following the treatment of anemia.
